SEQUENCE LISTING (52 sequences)

SEQ ID NO: 1 - DNA, 31 bp, Artificial Sequence
  Primer
ggatccatgggtcgcctcttattcctagtgc

SEQ ID NO: 2 - DNA, 41 bp, Artificial Sequence
  Primer
gcctaggtcacaaatcagtcttctcgttattcttctgtagc

SEQ ID NO: 3 - DNA, 35 bp, Artificial Sequence
  Primer
gagatctatggcccgtctcctcttcatactaccag

SEQ ID NO: 4 - DNA, 40 bp, Artificial Sequence
  Primer
gcctaggttacaaatcagtcttgacattcttcttctgcag

SEQ ID NO: 5 - DNA, 37 bp, Artificial Sequence
  Primer
ggatccatgtggctagaaatcctcctcacttcagtgc

SEQ ID NO: 6 - DNA, 26 bp, Artificial Sequence
  Primer
gcctaggtcattgccgctccagcacc

SEQ ID NO: 7 - DNA, 27 bp, Artificial Sequence
  Primer
ggatccatgtccgctccgttcgccaag

SEQ ID NO: 8 - DNA, 35 bp, Artificial Sequence
  Primer
cctaggctacttctgccacacctgctcgacaaatg

SEQ ID NO: 9 - DNA, 33 bp, Artificial Sequence
  Primer
ggatccatggcactcgcttacagcaacattccc

SEQ ID NO: 10 - DNA, 42 bp, Artificial Sequence
  Primer
cctaggtcattttctaccagcccatacttgttcacagaacgc

SEQ ID NO: 11 - DNA, 29 bp, Artificial Sequence
  Primer
tggatccatgtcgtattcagaccttcccc

SEQ ID NO: 12 - DNA, 35 bp, Artificial Sequence
  Primer
tgctagctcagtaattacctttgtacttctcccac

SEQ ID NO: 13 - DNA, 24 bp, Artificial Sequence
  Primer
agatctatgcccgcccgctcgctc

SEQ ID NO: 14 - DNA, 33 bp, Artificial Sequence
  Primer
tcctaggctacgatttttgctcctgagagagag

SEQ ID NO: 15 - DNA, 21 bp, Artificial Sequence
  Primer
gtggatccatggcgacacaca

SEQ ID NO: 16 - DNA, 22 bp, Artificial Sequence
  Primer
gacctaggctacttctcccaca

SEQ ID NO: 17 - DNA, 23 bp, Artificial Sequence
  Primer
gattaatgatcaatgagcgagca

SEQ ID NO: 18 - DNA, 20 bp, Artificial Sequence
  Primer
gacctaggtcacgacgacag

SEQ ID NO: 19 - DNA, 19 bp, Artificial Sequence
  Primer
gtggatccatggctgccca

SEQ ID NO: 20 - DNA, 18 bp, Artificial Sequence
  Primer
gagctagctcaggcctgg

SEQ ID NO: 21 - DNA, 31 bp, Artificial Sequence
  Primer
gggatccatggcaattcgacgtccagaagac

SEQ ID NO: 22 - DNA, 31 bp, Artificial Sequence
  Primer
gcctaggctagcggaaagcggtctttattcg

SEQ ID NO: 23 - DNA, 19 bp, Artificial Sequence
  Primer
gtggatccatgagcgagca

SEQ ID NO: 24 - DNA, 6578 bp, Artificial Sequence
  expression vector
tcatgtttgacagcttatcatcgatagagaccgggttggcggcgtatttgtgtcccaaaaaacagccccaattgccccaattgaccccaaattgacccagtagcgggcccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaggatccggtacctagggtgtctgtggtatctaagctatttatcactctttacaacttctacctcaactatctactttaataaatgaatatcgtttattctctatgattactgtatatgcgttcctctaagacaaatcgaatttcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagcagatccgcggccgcataggccactagtggatctgctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcatgtcacacaaaccgatcttcgcctcaaggaaacctaattctacatccgagagactgccgagatctgttcggaaatcaacggatgctcaaccgatttcgacagtaataatttgaatcgaatcggagcctaaaatgaacccgagtatatctcataaaattctcggtgagaggtctgtgactgtcagtacaaggtgccttcattatgccctcaaccttaccatacctcactgaatgtagtgtacctctaaaaatgaaatacagtgccaaaagccatggcactgagctcgtctaacggacttgatatacaaccaattaaaacaaatgaaaagaaatacagttctttgtatcatttgtaacaattaccctgtacaaactaaggtattgaaatcccacaatattcccaaagtccacccctttccaaattgtcatgcctacaactcatataccaagcactaacctaccaaacaccactaaaaccccacaaaatatatcttaccgaatatacagtaacaagctaccaccacactcgttgggtgcagtcgccagcttaaagatatctatccacatcagccacaactcccttcctttaataaaccgactacacccttggctattgaggttatgagtgaatatactgtagacaagacactttcaagaagactgtttccaaaacgtaccactgtcctccactacaaacacacccaatctgcttcttctagtcaaggttgctacaccggtaaattataaatcatcatttcattagcagggcagggccctttttatagagtcttatacactagcggaccctgccggtagaccaacccgcaggcgcgtcagtttgctccttccatcaatgcgtcgtagaaacgacttactccttcttgagcagctccttgaccttgttggcaacaagtctccgacctcggaggtggaggaagagcctccgatatcggcggtagtgataccagcctcgacggactccttgacggcagcctcaacagcgtcaccggcgggcttcatgttaagagagaacttgagcatcatggcggcagacagaatggtggcaatggggttgaccttctgcttgccgagatcgggggcagatccgtgacagggctcgtacagaccgaacgcctcgttggtgtcgggcagagaagccagagaggcggagggcagcagacccagagaaccggggatgacggaggcctcgtcggagatgatatcgccaaacatgttggtggtgatgatgataccattcatcttggagggctgcttgatgaggatcatggcggccgagtcgatcagctggtggttgagctcgagctgggggaattcgtccttgaggactcgagtgacagtctttcgccaaagtcgagaggaggccagcacgttggccttgtcaagagaccacacgggaagaggggggttgtgctgaagggccaggaaggcggccattcgggcaattcgctcaacctcaggaacggagtaggtctcggtgtcggaagcgacgccagatccgtcatcctcctttcgctctccaaagtagatacctccgacgagctctcggacaatgatgaagtcggtgccctcaacgtttcggatgggggagagatcggcgagcttgggcgacagcagctggcagggtcgcaggttggcgtacaggttcaggtcctttcgcagcttgaggagaccctgctcgggtcgcacgtcggttcgtccgtcgggagtggtccatacggtgttggcagcgcctccgacagcaccgagcataatagagtcagcctttcggcagatgtcgagagtagcgtcggtgatgggctcgccctccttctcaatggcagctcctccaatgagtcggtcctcgaacacaaactcggtgccggaggcctcagcaacagacttgagcaccttgacggcctcggcaatcacctcggggccacagaagtcgccgccgagaagaacaatcttcttggagtcagtcttggtcttcttagtttcgggttccattgtggatgtgtgtggttgtatgtgtgatgtggtgtgtggagtgaaaatctgtggctggcaaacgctcttgtatatatacgcacttttgcccgtgctatgtggaagactaaacctccgaagattgtgactcaggtagtgcggtatcggctagggacccaaaccttgtcgatgccgatagcgctatcgaacgtacccagccggccgggagtatgtcggaggggacatacgagatcgtcaagggtttgtggccaactggtaaataaatgatgactcaggcgacgacggaattc

SEQ ID NO: 25 - DNA, 21 bp, Artificial Sequence
  Primer
gaggatccatggagaagatag

SEQ ID NO: 26 - DNA, 24 bp, Artificial Sequence
  Primer
gacctaggttaaaacttttgatag

SEQ ID NO: 27 - DNA, 28 bp, Artificial Sequence
  Primer
gggatccatgacaaaatttgatatcaag

SEQ ID NO: 28 - DNA, 34 bp, Artificial Sequence
  Primer
gcctaggttatttagaatatttttcgaaaaaatc

SEQ ID NO: 29 - DNA, 25 bp, Artificial Sequence
  Primer
cctagggtgtctgtggtatctaagc

SEQ ID NO: 30 - DNA, 17 bp, Artificial Sequence
  Primer
ccgtctccgggagctgc

SEQ ID NO: 31 - DNA, 20 bp, Artificial Sequence
  Primer
catacaaccacacacatcca

SEQ ID NO: 32 - DNA, 21 bp, Artificial Sequence
  Primer
taaatagcttagataccacag

SEQ ID NO: 33 - DNA, 26 bp, Artificial Sequence
  Primer
aggatcctgttggattggaggattgg

SEQ ID NO: 34 - DNA, 20 bp, Artificial Sequence
  Primer
aggatccatggcgccctcag

SEQ ID NO: 35 - DNA, 22 bp, Artificial Sequence
  Primer
acctaggggtcttggaggtgtc

SEQ ID NO: 36 - DNA, 30 bp, Artificial Sequence
  Primer
tttaaatcgcttggcattagaagaagcagg

SEQ ID NO: 37 - DNA, 19 bp, Artificial Sequence
  Primer
gagggcgtcgactacgccg

SEQ ID NO: 38 - DNA, 22 bp, Artificial Sequence
  Primer
gtttaaaggcggcgacgagccg

SEQ ID NO: 39 - DNA, 23 bp, Artificial Sequence
  Primer
atcgatagagaccgggttggcgg

SEQ ID NO: 40 - DNA, 27 bp, Artificial Sequence
  Primer
aagcttttcgggtgtgagttgacaagg

SEQ ID NO: 41 - DNA, 27 bp, Artificial Sequence
  Primer
tcggatccggtacctagggtgtctgtg

SEQ ID NO: 42 - DNA, 31 bp, Artificial Sequence
  Primer
gaggatccttcgggtgtgagttgacaaggag

SEQ ID NO: 43 - DNA, 26 bp, Artificial Sequence
  Primer
cttccactgggccacaagcttttgtc

SEQ ID NO: 44 - DNA, 23 bp, Artificial Sequence
  Primer
agattgcgaggatcgtgccgagg

SEQ ID NO: 45 - DNA, 24 bp, Artificial Sequence
  Primer
agatctatgcccgcccgctcgctc

SEQ ID NO: 46 - DNA, 33 bp, Artificial Sequence; identical to SEQ ID NO: 14
  Primer

SEQ ID NO: 47 - protein, 20 aa, Artificial Sequence
  Synthetically generated peptide
ILAIARLVAAFKMATHTFAS

SEQ ID NO: 48 - protein, 20 aa, Artificial Sequence
  Synthetically generated peptide
EIPASSNAKRFKMATHTFAS

SEQ ID NO: 49 - protein, 20 aa, Artificial Sequence
  Synthetically generated peptide
SEAAVLQKRFGSMSEHSFEA

SEQ ID NO: 50 - protein, 20 aa, Artificial Sequence
  Synthetically generated peptide
SEAAVLQKRFGSMATHTFAS

SEQ ID NO: 51 - protein, 20 aa, Artificial Sequence
  Synthetically generated peptide
SEAAVLQKRFGSMAAHSFTA

SEQ ID NO: 52 - DNA, 20 bp, Artificial Sequence
  Primer
gagaattctcacgacgacag

(19) United States
(12) Patent Application Publication
(10) Pub. No.: US 2008/0171359 A1
(43) Pub. Date: Jul. 17, 2008

(54) Recombinant Yeasts for Synthesizing Epoxide Hydrolases

(75) Inventors: Adriana Leonora Botes, Edgeley (GB); Michel Labuschagne, Reddersburg (ZA); Robyn Roth, Sandton (ZA); Robin Kumar Mitra, Edgeley (GB); Jeanette Lotter, Edenvale Ridge (ZA); Rajesh Lalloo, Midrand (ZA); Deepak Ramduth, Centurion (ZA); Neeresh Rohitlall, Midrand (ZA); Clinton Simpson, Kempton Park (ZA); Petrus Van Zyl, Pretoria (ZA)

Correspondence Address: FISH & RICHARDSON P.C., P.O. BOX 1022, MINNEAPOLIS, MN, 55440-1022, US

(73) Assignee: Oxyrane (UK) Ltd., Manchester (GB)

(21) Appl. No.: 11/872,496

(22) Filed: Oct. 15, 2007

Related U.S. Application Data
(63) Continuation of application No. PCT/IB2006/002744, filed on Apr. 14, 2006.

(30) Foreign Application Priority Data
Apr. 14, 2005 (ZA) 2005/03031

(51) Int. Cl. C12P 21/00; C12N 1/19; C12N 9/14; C12N 15/63

(52) U.S. Cl. 435/69.1; 435/254.2; 435/254.22; 435/195; 435/320.1

(57) ABSTRACT

The invention provides isolated Y. lipolytica cells and substantially pure cultures of Y. lipolytica cells containing exogenous nucleic acids encoding EH polypeptides, e.g., enantioselective EH polypeptides. Also featured by the invention are methods for the production of the EH polypeptides and methods for hydrolysing epoxides and for producing optically active vicinal diols and/or optically active epoxides. Also embodied by the invention are efficient integrative expression vectors.

This application claims priority of South African Provisional Application No. 2005/03031, filed Apr. 14, 2005, the disclosure of which is incorporated herein by reference in its entirety.

### TECHNICAL FIELD

This invention relates to recombinant yeast strains, and more particularly to recombinant yeast strains containing exogenous epoxide hydrolase encoding nucleic acids.

### BACKGROUND

Epoxide hydrolases (EC 3.3.2.3; EH) are hydrolytic enzymes that convert epoxides to vicinal diols by ring-opening of the epoxide. Epoxide hydrolases are present in mammals, vertebrates, invertebrates, plants, insects, and microorganisms.

Optically active epoxides and vicinal diols are versatile fine chemical intermediates useful for the production of pharmaceuticals, agrochemicals, ferro-electric liquid crystals and flavours and fragrances. Epoxides are highly reactive electrophiles because of the strain inherent in the three-membered ring and the electronegativity of the oxygen. Epoxides react readily with various O-, N-, S-, and C-nucleophiles, acids, bases, reducing and oxidizing agents, allowing access to bi-functional molecules. Vicinal diols, employed as their highly reactive cyclic sulfites and sulfates, act like epoxide-like synthons with a broad range of nucleophiles. The possibility of double nucleophilic displacement reactions with amidines and azides allow access to dihydroimidazole derivatives, aziridines, diamines and diazides. Since enantiopure epoxides and vicinal diols can be stereospecifically inter-converted, they can be regarded as synthetic equivalents.

Major groups of substrate types that can be enantiomerically be resolved by epoxide hydrolases include mono-substituted epoxides (type I), styrene oxide-type oxiranes (type II), di-substituted epoxides (type III), tri-substituted, and tetra-substituted epoxides (type IV) [FIG. 1]. These substrates have enormous importance in the pharmaceutical, agrochemical and food industries. Examples of specific epoxides substrates are listed in International Application Nos. PCT/IB2005/001021, PCT/IB2005/001022, PCT/IB2005/001034 and PCT/IB2006/050143, as well as in South African Provisional Application Nos. 2005/03030 and 2005/03083, the disclosures of all of which are incorporated herein by reference in their entirety.

Epoxide hydrolases (EH) play crucial roles in the metabolism of organisms and as such are important drug targets in mammals. In addition, potentially important targets in the control of diseases of mammals and plants caused by parasites and microorganisms, as well as in the control of insects, both as carriers of parasites infecting humans and to protect crops against insect pests.

In order to exploit the diverse and ever increasing number of epoxide hydrolases for biocatalytic purposes and also to produce correctly folded epoxide hydrolases for the structure-function studies required for evaluation of these important metabolic enzymes as targets for therapeutic bioactive molecules, a generic expression system is highly desirable. However, at present no single expression system has been developed that can express functionally-active epoxide hydrolases from the all the various animal, plant, insect and microbial sources currently available.

### SUMMARY

The invention is based in part on the discovery by the inventors that recombinant Yarrowia lipolytica cells expressing exogenous EH from a wide range of species have high activity and, where the EH produced by the parent species is enantioselective, are also enantioselective. Thus, the invention provides isolated Y. lipolytica cells and substantially pure cultures of Y. lipolytica cells containing exogenous nucleic acids encoding EH, e.g., enantioselective EH. Also featured by the invention are methods for the production of the EH and methods for hydrolysing epoxides and for producing optically active vicinal diols and/or optically active epoxides. Also embodied by the invention are efficient integrative expression vectors.

In one aspect, the invention features a substantially pure culture of Yarrowia lipolytica cells, a substantial number of which comprise an exogenous nucleic acid encoding an epoxide hydrolase (EH) polypeptide. The invention also features an isolated Yarrowia lipolytica cell comprising an exogenous nucleic acid encoding an epoxide hydrolase (EH) polypeptide. It is understood that all of the embodiments described below for the cells of a substantially pure culture of cells apply also to an isolated cell.

The exogenous nucleic acid can be a vector, e.g., a vector in which the EH polypeptide-coding sequence is operably linked to an expression control sequence. The vector can contain a constitutive promoter. The vector can contain the TEF constitutive promoter or the hp4d promoter. The vector can be maintained as an episome in the cells or it can be fully integrated into the genome of the cells. The vector can contain an integration-targeting sequence and the genome of host cells to be transformed with the vector can contain an integration target sequences that is completely or partially homologous to the integration-targeting sequence. The integration-target sequence can be, for example, all or part of pBR322 plasmid. The vector can be the pKOV136 vector (Accession no.: ______).

The EH polypeptide encoded by the vector can be, for example, a bacterial, an insect, a plant, or a mammalian EH polypeptide. Moreover, the EH polypeptide can be a fungal polypeptide, e.g., a yeast yeast polypeptide. The yeast from which the EH is derived can be of any of the following genera: Arxula, Brettanomyces, Bullera, Bulleromyces, Candida, Cryptococcus, Debaryomyces, Dekkera, Exophiala, Geotrichum, Hormonema, Issatchenkia, Kluyveromyces, Lipomyces, Mastigomyces, Myxozyma, Pichia, Rhodosporidium, Rhodotorula, Sporidiobolus, Sporobolomyces, Trichosporon, Wingea, or Yarrowia. The yeast can be of any of the following species: Arxula adeninivorans, Arxula terrestris, Brettanomyces bruxellensis, Brettanomyces naardenensis, Brettanomyces anomalus, Brettanomyces species (e.g., Unidentified species NCYC 3151), Bullera dendrophila, Bulleromyces albus, Candida albicans, Candida fabianii, Candida glabrata, Candida haemulonii, Candida intermedia, Candida magnoliae, Candida parapsilosis, Candida rugosa, Candida tenuis, Candida tropicalis, Candida famata, Candida kruisei, Candida sp. (new) related to C. sorbophila, Cryptococcus albidus, Cryptococcus amylolentus, Cryptococcus bhutanensis, Cryptococcus curvatus, Cryptococcus gastricus, Cryptococcus humicola, Cryptococcus hungaricus, Cryptococcus laurentii, Cryptococcus luteolus, Cryptococcus macerans, Cryptococcus podzolicus, Cryptococcus terreus, Debaryomyces hansenii, Dekkera anomala, Exophiala dermatitidis, Geotrichumi spp. (e.g., Unidentified species UOFS Y-0111), Hormonema spp. (e.g., Unidentified species NCYC 3171), Issatchenkia occidentalis, Kluyveromyces marxianus, Lipomyces spp. (e.g., Unidentified species UOFS Y-2159), Lipomyces tetrasporus, Mastigomyces philipporii, Myxozyma melibiosi, Pichia anomala, Pichia finlandica, Pichia guillermondii, Pichia haplophila, Rhodosporidium lusitaniae, Rhodosporidium paludigenum, Rhodosporidium sphaerocarpum, Rhodosporidium toruloides, Rhodosporidium paludigenum, Rhodotorula araucariae, Rhodotorula glutinis, Rhodotorula minuta, Rhodotorula minuta var. minuta, Rhodotorula mucilaginosa, Rhodotorula philyla, Rhodotorula rubra, Rhodotorula spp. (e.g., Unidentified species NCYC 3193, UOFS Y-2042, UOFS Y-0448, UOFS Y-0139, UOFS Y-0560), Rhodotorula aurantiaca, Rhodotorula spp. (e.g., Unidentified species NCYC 3224), Rhodotorula sp. “mucilaginosa”, Sporidiobolus salmonicolor, Sporobolomyces holsaticus, Sporobolomyces roseus, Sporobolomyces tsugae, Trichosporon beigelii, Trichosporon cutaneum var. cutaneum, Trichosporon delbrueckii, Trichosporon jirovecii, Trichosporon mucoides, Trichosporon ovoides, Trichosporon pullulans, Trichosporon spp. (e.g., Unidentified species NCYC 3210, NCYC 3212, NCYC 3211, UOFS Y-0861, UOFS Y-1615, UOFS Y-0451, UOFS Y-0449, UOFS Y-2113), Trichosporon moniliiforme, Trichlosporon montevideense, Wingea robertsiae, or Yarrowia lipolytica.

The EH can be an enantioselective EH. Moreover, it can be a full-length EH or a functional fragment of a full-length EH.

The invention also features a method of producing an EH polypeptide, wherein the above-described culture of cells is cultured under conditions that are favorable for expression of the EH polypeptide. The method can provide expression resulting in a biomass-specific EH activity higher than the biomass-specific EH activity for cells that endogenously express the EH polypeptide. The EH polypeptide produced by this method can be secreted from the cells or it can be substantially not secreted by the cells during the culture. The EH polypeptide produced by the method can be recovered from the culture medium or from the cells.

This invention also features compositions of dry Yarrowia lipolytica cells, of which a substantial number contain an exogenous nucleic acid encoding an EH polypeptide. The composition can be made dry by freeze-drying, spray drying, fluidized bed drying, or agglomeration. The composition can be a shelf-stable, dry biocatalyst composition suitable for biocatalytic resolution of racemic epoxides. The dry cell composition can be formulated with one or more stabilizing agents prior to drying. These stabilizing agents can be a salt, a sugar, a protein, or an inert carrier. The stabilizing agent can be KCl. It is understood that the stabilizing agents can be used alone or in combination.

The invention also provides a method of hydrolysing an epoxide. This method involves the following steps: (a) providing an epoxide sample; (b) creating a reaction mixture by mixing a Y. lipolytica cellular EH biocatalytic agent with the epoxide sample; and (c) incubating the reaction mixture. The epoxide sample can be an enantiomeric mixture of an optically active expoxide and the Y. lipolytica cellular EH biocatalytic agent can be enantioselective. The method can further involve recovering from the reaction mixture: (a) an enantiopure, or a substantially enantiopure, vicinal diol; (b) an enantiopure, or a substantially enantiopure, epoxide; or (c) an enantiopure, or a substantially enantiopure, vicinal diol and an enantiopure, or a substantially enantiopure, epoxide. Optically active epoxides can be, without limitation, monosubstituted epoxides, styrene oxides, 2,2-disbubstituted epoxides, 2,3-disbubstituted epoxides, trisubstituted epoxides, tetra-substituted epoxides, meso-epoxides, or glycidyl ethers.

The invention also features a vector containing the following elements: (a) an expression control sequence, (b) a constitutive promoter; and (c) an integration-targeting sequence. The constitutive promoter can be the TEF promoter. The integration-targeting sequence can be, for example, all, or part, of the nucleotide sequence of the pBR322 plasmid. The vector can be, for example, the PKOV136 vector (Accession No. ______).

A polypeptide (full-length or fragment) having “epoxide hydrolase activity” (e.g., an epoxide hydrolase) is one which has hydrolytic enzyme activity that converts one or more epoxides to corresponding one more vicinal diols by ring-opening of the epoxide.

For convenience, cells of the Yarrowia genus are generally referred to below as “Yarrowia cells,” “Yarrowia transformant cells”, etc.

As used herein, both “protein” and “polypeptide” are used interchangeably and mean any chain of amino acid residues, regardless of length or post-translational modification (e.g., glycosylation or phosphorylation).

As used herein, an EH polypeptide is a full-length (mature or immature) EH protein or a functional fragment of an full-length (mature) EH protein. EH polypeptides can include native or heterologous signal peptides.

As used herein, a “functional fragment” of an EH is a fragment of the EH that is shorter than the full-length, mature EH and has at least 20% (e.g., at least: 30%; 40%; 50%; 60%; 70%; 80%; 90%; 95%; 98%; 99%; 100%, or more) of the ability of the full-length, mature polypeptide to hydrolyse an epoxide of interest. As used herein, a “functional fragment” of an enantioselective epoxide hydrolase polypeptide is a fragment of the full-length mature polypeptide that is shorter than the full-length mature polypeptide and has at least 20% (e.g., at least: 30%; 40%; 50%; 60%; 70%; 80%; 90%; 95%; 98%; 99%; 100%, or more) of the ability of the full-length polypeptide to enantioselectively hydrolyse a racemic epoxide mixture of interest. Fragments of interest can be made either by recombinant, synthetic, or proteolytic digestive methods and tested for their ability to (enantioselectively) hydrolyse an epoxide of interest.

The term “enantiomer” herein refers to one of two molecules having identical chemical structure and composition but which are optical isomers (also known as optical stereoisomers) of each other. The term “stereoisomer” herein refers to one of two molecules that have the same connectivity of atoms but whose arrangement in space is different in each isomer. As used herein, the term “optically active” refers to any substance that rotates the plane of incident linearly polarized light. Viewing the light head-on, some substances rotate the polarized light clockwise (dextrorotatory) and some produce a counterclockwise rotation (levorotatory). This rotation of polarized light occurs in solutions of chiral molecules (e.g., certain epoxides and vicinal diols).

The term “stereoselective” or “stereoselectivity” refers to the preferential formation, or depletion, in a chemical reaction (e.g., an EH-mediated chemical reaction) of one stereoisomer over another. When the stereoisomers are enantiomers, the phenomenon is called enantioselectivity and is quantitatively expressed by the enantiomer excess. Reactions are termed stereoselective (or enantioselective where applicable) if the selectivity is (a) complete (100%) i.e., the reaction results in only one stereoisomer/enantiomer of the relevant reaction product; or (b) partial, i.e., the reaction results in a mixture of two stereoisomers/enantiomers of the relevant reaction product in which the relative molar amount of one stereoisomer/enantiomer is at least 50.1% (e.g., at least: 55%; 60%; 65%; 70%; 80%; 90%; 95%; 97%; 98%; or 99%) of the total molar amount of both stereoisomer/enantiomers. The selectivity may also be referred to semiquantitatively as high or low stereoselectivity (or enantioselectivity).

As used herein, the term “wild-type” as applied to a nucleic acid or polypeptide refers to a nucleic acid or a polypeptide that occurs in, or is produced by, respectively, a biological organism as that biological organism exists in nature.

The term “heterologous” as applied herein to a nucleic acid in a host cell or a polypeptide produced by a host cell refers to any nucleic acid or polypeptide (e.g., an EH polypeptide) that is not derived from a cell of the same species as the host cell. Accordingly, as used herein, “homologous” nucleic acids, or proteins, are those that are occur in, or are produced by, a cell of the same species as the host cell.

The term “exogenous” as used herein with reference to nucleic acid and a particular host cell refers to any nucleic acid that does not occur in (and cannot be obtained from) that particular cell as found in nature. Thus, a non-naturally-occurring nucleic acid is considered to be exogenous to a host cell once introduced into the host cell. It is important to note that non-naturally-occurring nucleic acids can contain nucleic acid subsequences or fragments of nucleic acid sequences that are found in nature provided the nucleic acid as a whole does not exist in nature. For example, a nucleic acid molecule containing a genomic DNA sequence within an expression vector is non-naturally-occurring nucleic acid, and thus is exogenous to a host cell once introduced into the host cell, since that nucleic acid molecule as a whole (genomic DNA plus vector DNA) does not exist in nature. Thus, any vector, autonomously replicating plasmid, or virus (e.g., retrovirus, adenovirus, or herpes virus) that as a whole does not exist in nature is considered to be non-naturally-occurring nucleic acid. It follows that genomic DNA fragments produced by PCR or restriction endonuclease treatment as well as cDNAs are considered to be non-naturally-occurring nucleic acid since they exist as separate molecules not found in nature. It also follows that any nucleic acid containing a promoter sequence and polypeptide-encoding sequence (e.g., cDNA or genomic DNA) in an arrangement not found in nature is non-naturally-occurring nucleic acid. A nucleic acid that is naturally-occurring can be exogenous to a particular cell. For example, an entire chromosome isolated from a cell of yeast x is an exogenous nucleic acid with respect to a cell of yeast y once that chromosome is introduced into a cell of yeast y.

It will be clear from the above that “exogenous” nucleic acids can be “homologous” or “heterologous” nucleic acids. In contrast, the term “endogenous” as used herein with reference to nucleic acids or genes (or proteins encoded by the nucleic acids or genes) and a particular cell refers to any nucleic acid or gene that does occur in (and can be obtained from) that particular cell as found in nature.

As an illustration of the above concepts, an expression plasmid encoding a Y. lipolytica EH that is transformed into a Y. lipolytica cell is, with respect to that cell, an exogenous nucleic acid. However, the EH coding sequence and the EH produced by it are homologous with respect to the cell. Similarly, an expression plasmid encoding a potato EH that is transformed into a Y. lipolytica cell is, with respect to that cell, an exogenous nucleic acid. In contrast, however the EH coding sequence and the EH produced by it are heterologous with respect to the cell.

The term “biocatalyst” refers herein to any agent (e.g., an EH, a recombinant Y. lipolytica cell expressing an EH, or a lysate or cell extract of such a cell) that initiates or modifies the rate of a chemical reaction in a living body, i.e., a biochemical catalyst. Herein, the term “biotransformation” is the chemical conversion of substances (e.g., epoxides) as by the actions of living organisms (e.g., Yarrowia cells), enzymes expressed therefrom, or enzyme preparations thereof.

As used herein, a “Y. lipolytica cellular EH biocatalytic agent” is an agent containing or consisting of either: (a) recombinant Y. lipolytica intact viable cells containing an exogenous nucleic acid that encodes an EH polypeptide; or (b) a subcellular fraction, lyaste, crude extract, or semi-purified extract of recombinant Y. lipolytica intact cells containing an exogenous nucleic acid that encodes an EH polypeptide

As used herein, a polypeptide or protein that is “secreted” is a one all, or some, of which is exported from the cell. The protein may be secreted from the cell through the use of a signal peptide. Although signal peptides display very little primary sequence conservation, they generally include 3 domains: (a) an N-terminal region containing amino acids which contribute a net positive charge, (b) a central hydrophobic block of amino acids, and (c) a C-terminal region which contains the cleavage site. The nucleotide sequences encoding signal peptides can be present as part of a DNA sequence naturally encoding the secreted protein, or they be genetically engineered to be part of the DNA sequence encoding the secreted protein. Where a signal peptide is a signal peptide that occurs in a protein as that protein occurs in nature, the signal peptide is referred to as a homologous signal peptide. On the other hand, where a signal peptide is a signal peptide that does not occur in a protein as that protein occurs in nature, the signal peptide is referred to as a heterologous signal peptide.

As used herein a polypeptide that is “substantially not secreted” by a cell is a protein produced by the cell, either none of which is secreted by the cell or a minority (i.e., less than 10% (e.g., less than: 8%; 7%; 5%; 4%; 3%; 2%; 1%;)) of the molecules of which are secreted by the cell. Such a protein can be one that does not include an appropriate signal sequence or peptide. Alternatively, a protein “substantially not secreted” by a cell can be a protein which contains a retention- or targeting signal that serves to retain or target the protein to a subcellular localization other than a secretion pathway (e.g., the cell nucleus, cell-membrane, or mitochondria in the cell).

As used herein, the term “operably linked”, as applied to a coding sequence of interest, means incorporated into a genetic construct so that an expression control sequence in the genetic construct effectively controls expression of the coding sequence.

As used herein, a “constitutive promoter” is an unregulated promoter that allows for continual transcription of its associated transcribed region (e.g., the TEF promoter). As used herein, “integration-target sequence” is a DNA sequence within a host cell genome, endogenous or exogenous to the host, that facilitates the integration of an exogenous nucleic acid (e.g., an expression vector), which includes a corresponding “integration-targeting sequence”, into the host cell genome. Generally the “integration-target sequence” and the “integration-targeting sequence” have significant homology (i.e., greater than: 70%; 75%; 80%; 85%; 90%; 95%; 98%; 99%; or even 100% homology).

As used herein, the term “episome” refers to an exogenous genetic element (e.g., a plasmid) in a cell (e.g., a yeast cell) that is not integrated into the genome of the cell and can replicate autonomously in the cytoplasm of the cell. Exogenous genetic elements can also “integrate” or be inserted into the genome of the cell and replicate with the genome of the cell.

“Substantially enantiopure” optically active epoxide (or vicinal diol) preparations are preparations in which the molar amount of the particular enantiomer of the epoxide (or vicinal diol) is at least 55% (e.g., at least: 60%; 70%; 80%; 85%; 90%; 95%; 97%; 98%; 99%; 99.5%; 99.8%; or 99.9%) of the total molar amount of both epoxide (or vicinal diol) enantiomers.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. In case of conflict, the present specification, including definitions, will control. In addition, the materials, methods, and examples are illustrative only and not intended to be limiting. All publications, patent applications, patents, and other references mentioned herein are incorporated by reference in their entirety. For example, International Application Nos. PCT/IB2005/001021, PCT/IB2005/001022, PCT/IB2005/001034 and PCT/IB2006/050143 as well as South African Provisional Application Nos. 2005/03030, 2005/03083, and 2005/03031 are incorporated herein by reference in their entirety.

Other features and advantages of the invention, e.g., a method of making EH using recombinant Y. lipolytica cells, will be apparent from the detailed description and from the claims.

### DESCRIPTION OF DRAWINGS

FIG. 1 is a depiction of different substrate types for microbial epoxide hydrolases: monosubstituted epoxide (type I); styrene oxide-type epoxide (type II); 2,2, disubstituted epoxides (type III) and tri- and tetrasubstituted epoxides (type IV). Tri- and tetra-substituted epoxides are shown together in (type IV); for tri-substituted epoxides any one of the R groups is H and for tetra-substituted epoxides none of the R groups is H.

FIG. 2 is a diagram showing the phylogenetic analysis (performed using DNAMAN, (Lynnon Corporation, Vandreuil-Dorion, Quebec, Canada), using observed divergency and 1000 Bootstrap trials) of deduced amino acid sequences of available mEH. The analysis indicated 4 major mEH groups of fungal (solid shading), insect (dotted shading), vertebrate (meshed shading) and bacterial (checkered shading) origin. All sequences, except for those starting with BD, can be traced using the NCBI accession numbers. The sequences starting with BD were obtained from Zhao et al. (2004).

FIG. 3 is a diagram showing the amino acid homology analysis of the EH used in the studies described herein. The different degrees of homology between the various EH are indicated as percentages at the points of divergence (%). The homology tree was constructed using DNAMAN (Lynnon Corporation).

FIG. 4 is a depiction of the vector (pKOV136) used generate for YL-sTsA transformants (YL=Yarrowia lipolytic expression host, −s=true Single copy, T=TEF promoter, s=single copy integration selection (ura3d1 marker) A=signal peptide Absent) and of how the vector was constructed.

FIG. 5 is a depiction of the upstream region of the XPR2^{p} promoter according to an analysis conducted by Madzak et al. (1999).

FIG. 6 is a depiction of the pre and pro (“pre-pro”) regions (including the signal peptides) of the XPR2 (A) and the LIP2 (B) coding sequences. The various types of shading indicate the different regions of the pre-pro peptides (indicated in the legend).

FIGS. 7A and 7B are line graphs showing the comparison of the relative activities (FIG. 7A) and selectivities (FIG. 7B) of YL-sTsA transformants expressing microsomal and cytosolic EH from different origins that was used to select the catalyst with the required kinetic properties under uniform conditions of expression.

FIG. 8 is a bar graph showing the initial rates of hydrolysis of racemic 1,2-epoxyoctane as well as the (R)- and (S)-enantiomers by YL-sTsA transformants expressing microsomal and cytosolic EH of different origins under uniform conditions of expression that allow the unbiased selection of the catalyst with the required kinetic properties.

FIG. 9 is a line graph showing a comparison of the selectivities of the native EH from Rhodotorula araucariae (#25, NCYC 3183) (WT-25) and that of the recombinant enzyme expressed in Y. lipolytica (YL-25-TsA) for different epoxides: 1,2-epoxyoctane (EO), styrene oxide (SO), the meso-epoxide cyclohexene oxide (CO) and 3-chlorostyrene oxide (3CSO).

FIGS. 10A-10D are line graphs showing a comparison of the hydrolysis of different epoxides (Styrene oxide FIG. 10A, Indene oxide FIG. 10B, 2-methyl-3-phenyl-1,2-epoxypropane FIG. 10C and cyclohexene oxide FIG. 10D) by the recombinant enzyme from Rhodotorula araucariae (#25) expressed in S. cerevisiae (SC-25) and Y. lipolytica (YL-25 TsA). In all cases the SC-25 transformants displayed a decrease in activity and selectivity compared to YL-25 sTsA transformants.

FIG. 11 is a photograph of a TLC (thin layer chromotography) analysis of a biotransformation using 1,2-epoxyoctane as a substrate for the recombinant EH from R. toruloides (#46) under control of the XPR2^{p} and containing the signal peptides from T. reesei endoglucanase I coding sequence (lanes 1 and 2) and the XPR2 prepro-region (lanes 3 and 4) as signal peptides to direct the protein to the extracellular environment. Lanes 1 and 2 and lanes 3 and 4 indicate the cellular and extracellular fractions respectively.

FIGS. 12A-D are photographs of a qualitative TLC analysis of a biotransformation using 1,2-epoxyoctane as substrate for the recombinant EH produced by Po1h strains transformed using the multiple copy system (pINA1293) containing the EH coding sequences from R. araucariae (YL-25 HmL) (A), R. toruloides (YL-46 HmL) (B), R. paludigenum (YL-692 HmL) (C) and the negative control (D). The biotransformations were carried out using both a 20% (m/v) cellular suspension and supernatant from each 24 hour sample taken after stationary growth phase for a total time of 7 days (lanes 1-7).

FIG. 13 is a line graph showing a comparison of the hydrolysis of 1,2-epoxyoctane by the native EH from R. toruloides (WT-46) with that of the recombinant enzyme expressed with the T. reesei signal peptide (YL-46 XRP) and with the Y. lipolytica LIP2 signal peptide (YL-46 HmL).

FIG. 14 is a line graph showing a comparison of the hydrolysis of 1,2-epoxyoctane by the native EH from R. toruloides (WT-46) with that of the recombinant, enzyme expressed without a signal peptide in Y. lipolytica (YL-46 TsA).

FIG. 15 is a line graph showing a comparison of the hydrolysis of 1,2-epoxyoctane by the EH from R. araucariae (#25) expressed in the wild type (WT-25), and the recombinant enzyme expressed in Y. lipolytica with a signal peptide (YL-25 HmL) retained intracellularly (YL-25 HmL cells) and secreted into the supernatant (YL-25 HmL SN). The whole cell biotransformations were carried out with 20% (w/v) cellular suspensions in 10 ml reaction volume, while the biotransformation with the SN was carried out using the entire SN fraction from a 25 ml shake flask from which the cells were harvested and concentrated by ultrafiltration to 10 ml reaction volume.

FIG. 16 is a set of line graphs showing a comparison of the hydrolysis of 1,2-epoxyoctane by the recombinant EH from different wildtype yeasts expressed in Y. lipolytica with (YL-HmL transformants) and without (YL-HmA and YL-TsA transformants) a secretion signal all under control of the hp4d promoter but employing either multi-copy (HmL and HmA) or single copy (TsA) integrative vectors.

FIG. 17 is a set of line graphs showing a comparison of the hydrolysis of styrene oxide by the recombinant EH from different source yeasts expressed in Y. lipolytica with (YL-HmL transformants) and without (YL-HmA and YL-TsA transformants) a secretion signal all under control of the hp4d promoter but employing either multi-copy (HmL and HmA) or single copy (TsA) integrative vectors.

FIG. 18 is a set of line graphs showing a comparison of the hydrolysis of 3-chlorostyrene oxide by the recombinant EH from different source yeasts expressed in Y. lipolytica with (YL-HmL transformants) and without (YL-HMA and YL-TsA transformants) a secretion signal all under control of the hp4d promoter but employing either multi-copy (HmL and HmA) or single copy (TsA) integrative vectors.

FIG. 19 is a set of line graphs showing a comparison of the hydrolysis of the meso-epoxide cyclohexene oxide by the recombinant EH from different source yeasts expressed in Y. lipolytica with (YL-HmL transformants) and without (YL-HmA and YL-TsA transformants) a secretion signal all under control of the hp4d promoter but employing either multi-copy (HmL and HmA) or single copy (TsA) integrative vectors.

FIG. 20 is a set of line graphs showing a comparison of the hydrolysis of indene oxide by the recombinant EH from #692 (R. paludigenum NCYC 3179) expressed in Y. lipolytica with (YL-692 HmL transformant) and without (YL-692 HmA transformant) a secretion signal under all control of the hp4d promoter employing multi-copy (HmL and HmA) integrative vectors. The biotransformations were conducted at 20° C., pH 7.5 using 10% wet weight cells/volume (equivalent to 2% dry weight/volume).

FIG. 21 is a set of line graphs shows a comparison of the hydrolysis of 2-methyl-3-phenyl-1,2-epoxypropane by the recombinant EH from #692 (R. paludigenum NCYC 3179) expressed in Y. lipolytica with (YL-692 HmL transformant) and without (YL-692 HmA transformant) a secretion signal all under control of the hp4d promoter employing multi-copy (HmL and HmA) integrative vectors.

FIG. 22 is a set of line graphs showing the resolution of 1,2-epoxyoctane by YL-TsA and YL-HmA transformants harboring the EH from #692 (R. paludigenum NCYC 3179) and #777 (C. neoformans CBS 132). For YL-TsA transformants, 10% wet weight cells (equal to 2% dry weight) was used, while half the biomass concentration (5% wet weight=1% dry weight) was used for YL HmA transformants. For #692, the YL-HmA transformant displayed double the activity observed for the YL-TsA transformant and the selectivity remained unchanged. For # 777, an increase in both activity and selectivity of the YL-HmA transformant compared to that of the YL-TsA transformant was observed.

FIG. 23 is a set of line graphs showing the resolution of styrene oxide by YL-TsA and YL-HmA transformants harboring the EH from #46 (R. toruloides UOFS Y-0471) and #692 (R. paludigenum NCYC 3179). For YL-TsA transformants, 20% wet weight cells (equal to 4% dry weight) was used, while half the biomass concentration (10% wet weight=2% dry weight) was used for YL HrnA transformants. For both #46 and #692, the activity of the YL-HmA and YL-TsA transformants remained essentially unchanged, while a significant increase in selectivity (2× for #46 and >5× for #692) was observed for both EH expressed in the YL-HMA transformants compared to the YL-TsA transformants.

FIG. 24 is a set of line graphs showing the resolution of phenyl glycidyl ether by YL-TsA and YL-HmA transformants harboring the EH from #46 (R. toruloides UOFS Y-0471) and #692 (R. paludigenum NCYC 3179). For both YL-TsA and YL-HmA transformants, 10% wet weight cells (equal to 2% dry weight) was used. For both #46 and #692, the selectivity of the YL-HMA and YL-TsA transformants remained essentially unchanged, while a significant increase in activity (2× for #46 and >5× for #692) was observed for both EH expressed in the YL-HmA transformants compared to the YL-TsA transformants.

FIG. 25 is a set of line graphs showing the resolution of indene oxide by YL-TsA and YL-HmA transformants harboring the EH from #692 (R. paludigenum NCYC 3179) #23 (R. mucilaginosa UOFS Y-0198). For YL-TsA transformants, 10% wet weight cells (equal to 2% dry weight) was used, while half the biomass concentration (5% wet weight=1% dry weight) was used for YL HmA transformants. For #692, the YL-HmA transformant displayed 7 times the activity observed for the YL-TsA transformant and the selectivity remained essentially unchanged. For #23, an increase in both activty and selectivity of the YL-HmA transformant compared to that of the YL-TsA transformant was observed.

FIGS. 26A and 26B are line graphs showing the resolution of styrene oxide by YL-HmA transformants harboring the coding sequences from the plant source Solanum. tuberosum (FIG. 26A) and from the yeast R. paludigenum (#692) (FIG. 26B). The S. tuberosum YL-HmA transformant displayed the same excellent enantioselectivity on the substrate as reported for the native gene (expressed in Baculovirus and E. coli), which is opposite to that of yeast epoxide hydrolases. Activity of the S. tuberosum construct in Yarrowia was essentially identical to that obtained for YL-692 HmA.

FIG. 27 is a line graph showing the resolution of styrene oxide by the YL-HmA transformant harboring the coding sequence from the bacterium Agrobacterium radiobacter. The A. radiobacter Yarrowia HmA transformant displayed the same selectivity as reported for the native coding sequence over-expressed in A. radiobacter.

FIG. 28 is a photomicrograph showing Yarrowia lipolytica (YL-25 HmA) cells.

FIG. 29 is a line graph showing the effect of sugar feed rate on the growth of Y. lipolytica (YL25 HmA). Ep 07-04, Ep 08-04 and Ep 09-04 refer to specific glucose feed rates of 3.8, 14.5 and 5.0 gram glucose per litre initial batch broth volume per hour respectively.

FIG. 30 is a line graph showing the effect of sugar feed rate on the specific enzyme activity of Y. lipolytica (YL25HmA). Ep 07-04, Ep 08-04 and Ep 09-04 refer to specific glucose feed rates of 3.8, 14.5 and 5.0 gram glucose per litre initial batch broth volume per hour respectively.

FIG. 31 is a line graph showing the effect of sugar feed rate on the volumetric enzyme activity of Y. lipolytica (YL25 HmA). Ep 07-04, Ep 08-04 and Ep 09-04 refer to specific glucose feed rates of 3.8, 14.5 and 5.0 gram glucose per litre initial batch broth volume per hour respectively.

FIG. 32 is a line graph showing the effect of specific growth rates on the specific intracellular epoxide hydrolase production during the fermentation of Y. lipolytica (YL25 HmA). Ep 07-04, Ep 08-04 and Ep 09-04 refer to specific glucose feed rates of 3.8, 14.5 and 5.0 gram glucose per litre initial batch broth volume per hour respectively.

FIG. 33 is a depiction of the nucleotide sequence (SEQ ID NO:24) of the PKOV136 expression vector. The sequence of the pBR322 plasmid-derived integration target sequence integrated into the genome of Yarrowia lipolytica strain Po1g is underlined. The non-underlined sequence within the underlined sequence is not in the integration-target sequence in the genome of the Po1G strain

### DETAILED DESCRIPTION

The present invention relates to the use of yeast cells (i.e., Yarrowia yeast cells such as Y. lipolytica cells) as a recombinant expression system for use either as a whole cell, or cell extract or lysate, biocatalyst exhibiting epoxide hydrolase (EH) activity, or for the production of a polypeptide exhibiting epoxide hydrolase activity, of microbial, animal, insect or plant origin that can used as a biocatalyst.

The expression systems that can be used for purposes of the invention include, but are not limited to, microorganisms such as yeasts (e.g., any of the genera, species or strains listed herein) or bacteria (e.g., E. coli and B. subtilis) transformed with recombinant bacteriophage DNA, plasmid DNA, or cosmid DNA expression vectors containing the nucleic acid molecules of the invention; yeast (for example, Saccharomyces, Kluyveromyces, Hansenula, Pichia, Yarrowia, Arxula and Candida, and other genera, species, and strains listed herein) cells transformed with recombinant yeast expression vectors containing the nucleic acid molecule of the invention; insect cell systems infected with recombinant virus expression vectors (for example, baculovirus) containing the nucleic acid molecule of the invention; plant cell systems infected with recombinant virus expression vectors (for example, cauliflower mosaic virus (CaMV) or tobacco mosaic virus (TMV)) or transformed with recombinant plasmid expression vectors (for example, Ti plasmid) containing a YESH nucleotide sequence; or mammalian cell systems (for example, COS, CHO, BHK, 293, VERO, HeLa, MDCK, WI38, and NIH 3T3 cells) harboring recombinant expression constructs containing promoters derived from the genome of mammalian cells (for example, the metallothionein promoter) or from mammalian viruses (for example, the adenovirus late promoter and the vaccinia virus 7.5K promoter). Also useful as host cells are primary or secondary cells obtained directly from a mammal and transfected with a plasmid vector or infected with a viral vector.

The invention includes a recombinant Y. lipolytica cell containing an exogenous nucleic acid (e.g., DNA) encoding an EH. The cells are preferably isolated cells. As used herein, the term “isolated” as applied to a microorganism (e.g., a yeast cell) refers to a microorganism which either has no naturally-occurring counterpart (e.g., a recombinant microorganism such as a recombinant yeast) or has been extracted and/or purified from an environment in which it naturally occurs. Thus, an “isolated microorganism” does not include one residing in an environment in which it naturally occurs, for example, in the air, outer space, the ground, oceans, lakes, rivers, and streams and the like, ground at the bottom of oceans, lakes, rivers, and streams and the like, snow, ice on top of the ground or in/on oceans lakes, rivers, and streams and the like, man-made structures (e.g., buildings), or in natural hosts (e.g., plant, animal or microbial hosts) of the microorganism, unless the microorganism (or a progenitor of the microorganism) was previously extracted and/or purified from an environment in which it naturally occurs and subsequently returned to such an environment or any other environment in which it can survive. An example of an isolated microorganism is one in a substantially pure culture of the microorganism.

Moreover the invention provides a substantially pure culture of Y. lipolytica cells, a substantial number (i.e., at least 40% (e.g., at least: 50%; 60%; 70%; 80%; 85%; 90%; 95%: 97%; 98%; 99%; 99.5%; or even 100%) of which contain an exogenous nucleic acid encoding an epoxide hydrolase. As used herein, a “substantially pure culture” of a microorganism is a culture of that microorganism in which less than about 40% (i.e., less than about: 35%; 30%; 25%; 20%; 15%; 10%; 5%; 2%; 1%; 0.5%; 0.25%; 0.1%; 0.01%; 0.001%; 0.0001%; or even less) of the total number of viable microbial (e.g., bacterial, fungal (including yeast), mycoplasmal, or protozoan) cells in the culture are viable microbial cells other than the microorganism. The term “about” in this context means that the relevant percentage can be 15% percent of the specified percentage above or below the specified percentage. Thus, for example, about 20% can be 17% to 23%. Such a culture of microorganisms includes the microorganisms and a growth, storage, or transport medium. Media can be liquid, semi-solid (e.g., gelatinous media), or frozen. The culture includes the cells growing in the liquid or in/on the semi-solid medium or being stored or transported in a storage or transport medium, including a frozen storage or transport medium. The cultures are in a culture vessel or storage vessel or substrate (e.g., a culture dish, flask, or tube or a storage vial or tube).

The microbial cells of the invention can be stored, for example, as frozen cell suspensions, e.g., in buffer containing a cryoprotectant such as glycerol or sucrose, as lyophilized cells. Alternatively, they can be stored, for example, as dried cell preparations obtained, e.g., by fluidised bed drying or spray drying, or any other suitable drying method. Similarly the enzyme preparations can be frozen, lyophilised, or immobilized and stored under appropriate conditions to retain activity.

Y. lipolytica is particularly useful in industrial applications due to its ability to grow on n-paraffins and produce high amounts of organic acids. The yeast is considered non-pathogenic and has been awarded “generally recognized as safe” (GRAS) status for several industrial processes. Y. lipolytica has an innate ability to synthesize and secrete significant quantities of several proteins into culture medium, specifically proteases, lipases, phosphatases, esterases and RNase. Thus, Y. lipolytica can be used to express and secrete a wide variety of heterologous proteins. See, e.g., Park et al., 2000; Nicaud et al., 2002; Müller et al., 1998; Park et al., 1997; Swennen et al., 2002; and Nicaud et al., 1989.

Any suitable promoter can be used to drive expression of a heterologous coding sequence in a yeast species such as Y. lipolytica. These include, without limitation, the Y. lipolytica inducible promoters XPR2^{p} (alkaline extracellular protease, inducible by peptones), ICL1^{p} (isocitrate lyase, inducible by fatty acids), POX2^{p} (acyl-coenzyme A oxidases, inducible by fatty acids) and POT1^{p} (thiolase, inducible by acetate) (see, e.g., Nicaud et al., 1989b; Le Dall et al., 1994; Park et al., 1997; and Pignède et al., 2000).

Other examples of useful promoters include, without limitation, constitutive promoters such as the ribosomal protein S7 promoter (RPS7^{p}) and the transcription elongation factor-1α promoter (TEF^{p}).

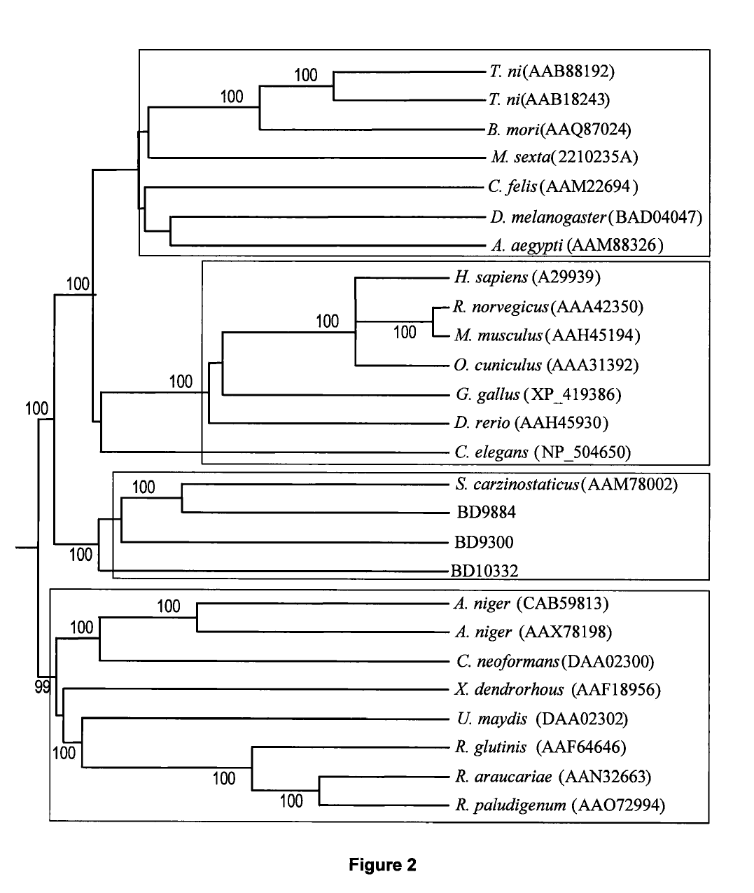

Synthetic hybrid promoters also can be used. For example, a promoter such as hp4d^{p} (Madzak et al., 1999) can contain four direct tandem copies of the upstream activating sequence 1 (UAS1B) from the native XPR2^{p} in front of a minimal LEU2^{p} also can be used. Other hybrid promoters can contain minimal forms of the POX2^{p} and XPR2^{p} in combination with the four tandem repeats of the UAS1B (see, e.g., Madzak et al., 2000). Analysis of the upstream regions of the XPR2^{p} revealed two activating sequences (UAS; FIG. 2) essential for promoter activity (Madzak et al., 1999). UAS1 and UAS2, can be further divided into UAS1A, UAS1B and UAS2A, UAS2B, UAS2C respectively. The UAS1A fragment is a 29 bp sequence beginning 805 bp upstream of the XPR2^{p} initiation site. This region, placed in front of a minimal LEU2^{p}, can promote an enhancement of activity. The UAS1B region, encompassing the whole of the UAS1A region with the addition of two imperfect repeats, can enhance activity even more than the UAS1A region, indicating the participation of the added region to the UAS effect.

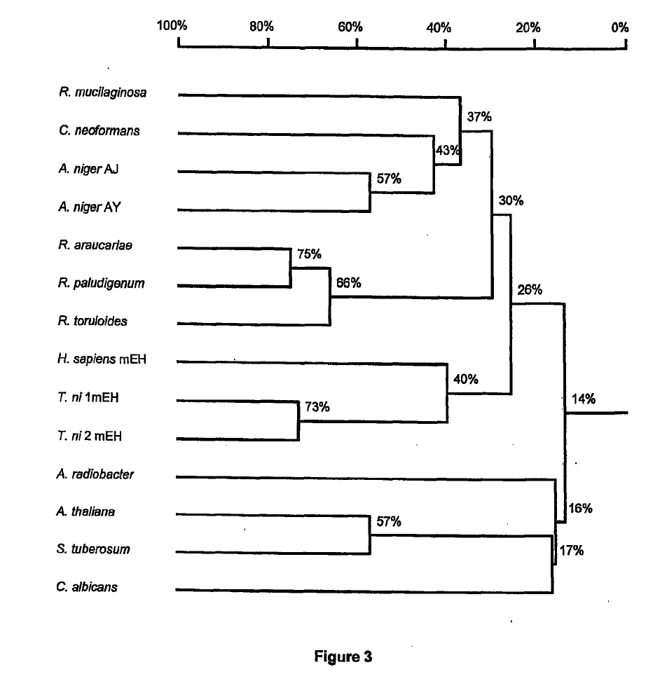

A EH polypeptide to be expressed in a yeast such as Y. lipolytica may or may not include a signal peptide that can guide the polypeptide to a location of interest. When included, any suitable signal peptide can be used. Suitable signal peptides include the polypeptide's own (autologous signal) peptide, a heterologous signal peptide, a signal peptide of another polypeptide naturally expressed by the host cell, or a synthetic (non-naturally occurring) signal peptide. Where non-wild-type signal peptides are added to a polypeptide, none, all, or part of the native (wild-type) signal can be included. Where some or all of the native signal peptide as well as non-wild-type signal are used, the initiator Met residue of the native signal peptide can, optionally, be deleted. For example, the signal peptide and the pre-pro region of the alkaline extracellular protease (AEP) (Nicaud et al., 1989a) can be included. This signal contains a short pre-region containing a 13-amino acid signal sequence and a stretch of ten dipeptides (motif X-Ala or X-Pro, where X is any amino acid) dipeptides followed by a relative large pro-region consisting of 1224 amino acids ending with a recognition site (Lys-Arg) for a KEX2-like endoprotease encoded by the XPR6 gene (Enderlin & Ogrydziak, 1994). The signal also contains a glycosylation site, and can act as a chaperone for AEP secretion (FIG. 6; Fabre et al., 1991; and Fabre et al., 1992). See also Matoba et al., 1997; and Park et al., 1997. The secretion signal of the extracellular lipase encoded by the LIP2 gene can also be included. The LIP2 secretion signal has features similar to the those of the XPR2 signal: a short sequence (13 amino acids) followed by four dipeptides (X-Ala/X-Pro, where X is any amino acid) (a possible site for processing by a diaminopeptidase), a short proregion (10 amino acids) and a LysArg cleavage site (a putative processing site for the KEX2-like endopeptidase encoded by the XPR6 gene) (FIG. 3B) (Pignède et al., 2000). A hybrid between the XPR2 and LIP2 prepro regions can also be used (Nicaud et al., 2002).

Further examples of useful signal peptides include, without limitation, the 22 amino acid signal peptide of the endoglucanase I coding sequence from T. reesei (Park et al., 2000) the rice α-amylase signal peptide (Chen et al., 2004).

Any expression vector that can accomplish integration into the genome of Y. lipolytica can also be used. For example, expression vectors that rely on the zeta elements from the retro-transposon Ylt1 to accomplish random non-homologous integration into the genome of Ylt1-devoid Y. lipolytica strains can be used in combination with markers that leads to the integration of variable numbers of expression cassettes into the genome. A constitutive site specific single copy integrative vector that allows for homologous, site-specific recombination in the genome of a recipient strain devoid of the Ylt1 retrotransposon can also be constructed.

Expression vectors containing integration-targeting sequences for homologous recombination can also be used. For use with such vectors, appropriate host cells should have genomes containing appropriate corresponding integration-target sequences for homologous integration within the selection marker for integration (e.g. in LEU, URA3, XPR2 terminator, rDNA and zeta sequences in Ylt1-carrying strains). The integration-target sequences can be exogenous nucleotide sequences stably incorporated into the genomes of the host cells (such as the pBR322 docking platform). They can be, for example, all or a part of the expression vector nucleotide sequence. Alternatively, an integration-targeting sequence in an appropriate expression vector can contain a nucleotide sequence derived from the genome of a host cell of interest (e.g., any of the host cells described herein). Y. lipolytica cells containing such integration-target sequences and vectors containing corresponding integration-targeting sequences are described below in Example 1 and Example 2. Integration target-sequences can be of variable nucleotide length generally ranging from 500 base pairs (0.5 kilobases (kb)) to 10 kb (e.g., 1-9 kb, 2-8 kb, or 3-7 kb).

One application of cloned EH polypeptide coding sequences of microbial, plant, insect and animal origin expressed intracellularly using a recombinant yeast (e.g., Y. lipolytica) strain pertains to their use as convenient systems for industrial application of the useful stereoselective and epoxide substrate specific properties demonstrated by some microbial, plant, insect and animal derived EH.

Another application of cloned soluble or microsomal EH coding sequences of microbial, plant, insect and mammalian origin expressed intracellularly using a recombinant yeast (e.g., Y. lipolytica) strain pertains to their use as convenient systems for the production of correctly folded (i.e. functional) protein for drug design. For example, high level expression of functional EH can facilitate the 3-D structure determination for “in silico” design of effectors (activators or inhibitors) of epoxide hydrolases. Furthermore, functionally expressed EH can be used to screen effectors for binding affinity and its inhibition or activation effects.

Another application of cloned soluble or microsomal EH coding sequences of microbial, plant, insect and mammalian origin expressed intracellularly using a recombinant yeast (e.g., Y. lipolytica) strain pertains to their use as convenient systems for the direct comparison of the characteristics of EH from different origins and environmental libraries, or the evaluation of new characteristics imparted to an EH by protein engineering techniques such as directed evolution or mutagenesis.

Polypeptides having EH activity include those for which genomic or cDNA sequences encoding these polypeptides or parts thereof can be obtained. For example, EH coding sequences can be obtained from microbial, plant, insect and animal genetic material (DNA or mRNA) and subsequently cloned, characterized and overexpressed intracellularly in Yarrowia host cells in accordance with one aspect of this invention. Appropriate organisms from which the EH polypeptide coding sequence can be obtained include, without limitation, animals (such as mammals, including, without limitation, humans, non-human primates, bovine animals, pigs, horses, sheep, goats, cats, dogs, rabbits, gerbils, hamsters, mice, or rats), insects (e.g., Drosophila), plants (e.g., tobacco or potato plants), or microorganisms (e.g., bacteria, fungi, including yeasts, mycoplasmas, or protozoans). Other genera, species, and strains of interest are recited below. The nucleotide sequences derived from the genetic material may also be mutated by site directed mutagenesis or random mutagenesis. not more 50 (e.g., not more than 50, 45, 40, 35, 30, 25, 20, 17, 14, 12, 10, nine, eight, seven, six, five, four, three, two, or one) conservative substitution(s). Mutagenesis techniques and other genetic engineering techniques such as the addition of poly-histidine (e.g., hexahistidine) tags to enable protein purification include techniques known to those skilled in the art. Also of interest are coding sequences encoding EH polypeptides containing not more 50 (e.g., not more than 50, 45, 40, 35, 30, 25, 20, 17, 14, 12, 10, nine, eight, seven, six, five, four, three, two, or one) conservative substitution(s). Conservative substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine. Moreover, the coding sequences can be recoded for host cell (e.g., Y. lipolytica host cell) codon bias.

Specifically pertaining to the use of EH polypeptides in the biocatalytic chiral resolution of racemic epoxides, the invention has application to the use of biocatalysts comprising any of a whole cell, part of a cell, a cell extract, or a cell lysate exhibiting a desired EH activity. Bio-resolution may be carried out for example in the presence of whole cells of the recombinant Yarrowia expression host or cultures thereof or preparations thereof comprising said polypeptide. These preparations can be, for example, crude cell extracts, or crude or pure enzyme preparations from said cell extracts. In cases where the polypeptide having EH activity is released by the recombinant Yarrowia host into the culture medium, either by, e.g., partial secretion or cell lysis, crude or purified preparations may also be obtained from the culture medium.

The EH polypeptides of microbial, insect, plant and animal origin for application as stereoselective biocatalysts are generally retained within the cell of the recombinant Yarrowia lipolytica strain for the purposes of ease of production of biocatalyst in high quantity. In general, Yarrowia (e.g., Y. lipolytica) recombinant strains can be cultured in an aqueous nutrient medium comprising sources of assimilatable nitrogen and carbon, typically under submerged aerobic conditions (shaking culture, submerged culture, etc.). The aqueous medium can be maintained at a pH of 5.0-6.5 using protein components in the medium, buffers incorporated into the medium or by external addition of acid or base as required. Suitable sources of carbon in the nutrient medium can include, for example, carbohydrates, lipids and organic acids such as glucose, sucrose, fructose, glycerol, starch, vegetable oils, petrochemical derived oils, succinate, formate and the like. Suitable sources of nitrogen can include, for example, yeast extract, Corn Steep Liquor, meat extract, peptone, vegetable meals, distillers solubles, dried yeast, and the like as well as inorganic nitrogen sources such as ammonium sulphate, ammonium phosphate, nitrate salts, urea, amino acids and the like.

Carbon and nitrogen sources, advantageously used in combination, need not be used in pure form because less pure materials, which contain traces of growth factors and considerable quantities of mineral nutrients, are also suitable for use. When desired, mineral salts such as sodium or potassium phosphate, sodium or potassium chloride, magnesium salts, copper salts and the like can be added to the medium. An antifoam agent such as liquid paraffin or vegetable oils may be added in trace quantities as required but is not typically required.

Cultivation of cells (e.g., Y. lipolytica cells) expressing an EH polypeptide can be performed under conditions that promote optimal biomass and/or enzyme titer yields. Such conditions include, for example, batch, fed-batch or continuous culture. For production of high amounts of biomass, submerged aerobic culture methods can be used, while smaller quantities can be cultured in shake flasks. For production in large tanks, a number of smaller inoculum tanks can be used to build the inoculum to a level high enough to minimise the lag time in the production vessel. The medium for production of the biocatalyst is generally be sterilised (e.g., by autoclaving) prior to inoculation with the cells. Aeration and agitation of the culture can be achieved by mechanical means simultaneous addition of sterile air or by addition of air alone in a bubble reactor.

EH polypeptides typically are retained within the cell of the recombinant cell (e.g., Yarrowia cell) for facile production of EH for biocatalytic purposes. Such intracellular production generally results in a EH biocatalyst exhibiting the most suitable kinetic characteristics for subsequent resolution of racemic epoxides. While use of the constitutive TEF and quasi-constitutive hp4d promoter systems do not require extraneous induction in order to induce enzyme production, inducible promoter systems may also be used and form an embodiment of this invention. After growth and suitable biocatalyst activity (as determined by standard methods) is obtained, cells can be harvested by conventional methods such as, for example, filtration or centrifugation and cell paste stored in a cryoprotectant-rich matrix (typically, but not limited to, glycerol) under chilled or frozen conditions until required for biotransformation. In one embodiment, the recombinant cells (e.g., Yarrowia cells) exhibiting EH activity can be harvested from the fermentation process by conventional methods such as filtration or centrifugation and formulated into a dry pellet or dry powder formulation while maintaining high activity and useful stereoselectivity. Processes for production of a dry powder whole cell biocatalyst exhibiting epoxide hydrolase activity can include spray-drying, freeze-drying, fluidised bed drying, vacuum drum drying, or agglomeration and the like. Drying methods such as freeze-drying, fluidised bed drying or a method employing extrusion/spheronisation pelleting followed by fluidised bed drying can be particularly useful. Temperatures for these processes may be <100° C. but typically <70° C. to maintain high residual activity and stereoselectivity. The dry powder formulation should have a water content of 0-10% w/w, typically 2-5% w/w. Stabilising additives such as salts (e.g. KCl), sugars, proteins and the like may be included to improve thermal tolerance or improve the drying characteristics of the biocatalyst during the drying process.

A harvested culture or formulated dry cell preparation may be manipulated to release the EH for further processing. For subsequent application in biocatalysis processes, a biocatalyst may be applied as a cell lysate or purified EH biocatalyst in the biotransformation, or may be used as whole cell preparation. For example, a biocatalyst can be used as a crude lysate or a whole cell catalyst for the stereoselective biotransformation of epoxides shown to be inhibitory or degradatory to the epoxide hydrolase activity. A biocatalyst can be used in any suitable aqueous buffer, typically in a phosphate buffer.

Immobilised or free whole cells or cell extracts, or crude or purified enzyme preparations may be used. Procedures for immobilisation of whole cells or enzyme preparations include those known in the art, and may include, for example, adsorption, covalent attachment, cross-linked enzyme aggregates or cross-linked enzyme crystals, and entrapment in hydrogels and into reverse micelles.

The application of microsomal and soluble EH biocatalysts to the hydrolyisis (and/or, where optically active, resolution) of epoxide substrates can, for example but without limitation, be accomplished using coding sequences isolated from the yeast genera Rhodosporidium and Rhodotorula and Candida, the bacterial genera Agrobacterium or Mycobacterium, the fungal genus Aspergillus, the plant genus Solanum, the insect genera Trichoplasia and Arabidopsis, and the mammalian genus Homo sapiens, which can be overexpressed intracellularly in recombinant Yarrowia (e.g., Y. lipolytica) and contacted with epoxides. Other yeast genera of interest include Arxula, Brettanomyces, Bullera, Bulleromyces, Cryptococcus, Debaryomyces, Dekkera, Exophiala, Geotrichum, Hormoenema, Issatchenkia, Kluyveromyces, Lipomyces, Mastigomyces, Myxozyma, Pichia, Sporidiobolus, Sporobolomyces, Trichosporon, Wingea, and Yarrowia. Yeast species of interest include, for example, Arxula adeninivorans, Arxula terrestris, Brettanomyces bruxellensis, Brettanomyces naardenensis, Brettanomyces anomalus, Brettanomyces species (e.g., Unidentified species NCYC 3151), Bullera dendrophila, Bulleromyces albus, Candida albicans, Candida fabianii, Candida glabrata, Candida haemulonii, Candida intermedia, Candida magnoliae, Candida parapsilosis, Candida rugosa, Candida tenuis, Candida tropicalis, Candida famata, Candida kruisei, Candida sp. (new) related to C. sorbophila, Cryptococcus albidus, Cryptococcus amylolentus, Cryptococcus bhutanensis, Cryptococcus curvatus, Cryptococcus gastricus, Cryptococcus humicola, Cryptococcus hungaricus, Cryptococcus laurentii, Cryptococcus luteolus, Cryptococcus macerans, Cryptococcus podzolicus, Cryptococcus terreus, Debaryomyces hansenii, Dekkera anomala, Exophiala dermatitidis, Geotrichum spp. (e.g., Unidentified species UOFS Y-0111), Hornonema spp. (e.g., Unidentified species NCYC 3171), Issatchenkia occidentalis, Kluyveromyces marxianus, Lipomyces spp. (e.g., Unidentified species UOFS Y-2159), Lipomyces tetrasporus, Mastigomyces philipporii, Myxozyma melibiosi, Pichia anomala, Pichia finlandica, Pichia guillermondii, Pichia haplophila, Rhodosporidium lusitaniae, Rhodosporidium paludigenum, Rhodosporidium sphaerocarpum, Rhodosporidium toruloides, Rhodosporidium paludigenum, Rhodotorula araucariae, Rhodotorula glutinis, Rhodotorula minuta, Rhodotorula minuta var. minuta, Rhodotorula mucilaginosa, Rhodotorula philyla, Rhodotorula rubra, Rhodotorula spp. (e.g., Unidentified species NCYC 3193, UOFS Y-2042, UOFS Y-0448, UOFS Y-0139, UOFS Y-0560), Rhodotorula aurantiaca, Rhodotorula spp. (e.g., Unidentified species NCYC 3224), Rhodotorula sp. “mucilaginosa”, Sporidiobolus salmonicolor, Sporobolomyces holsaticus, Sporobolomyces roseus, Sporobolomyces tsugae, Trichosporon beigelii, Trichosporon cutaneum var. cutaneum, Trichosporon delbrueckii, Trichosporon jirovecii, Trichosporon mucoides, Trichosporon ovoides, Trichosporon pullulans, Trichosporon spp. (e.g., Unidentified species NCYC 3210, NCYC 3212, NCYC 3211, UOFS Y-0861, UOFS Y-1615, UOFS Y-0451, UOFS Y-0449, UOFS Y-2113), Trichosporon moniliiforme, Trichosporon montevideense, Wingea robertsiae, and Yarrowia lipolytica (see International Application No. PCT/IB2005/001034)

A process for the production of epoxides and vicinal diols from epoxides employing recombinant Yarrowia lipolytica preparations (e.g., whole cells, cell extracts or crude or purified enzyme extracts) that contain a polypeptide of microbial, insect, plant and mammalian and invertebrate origin having EH activity, which can be free or immobilized, may typically be performed under very mild conditions. Preferably the epoxides and vicinal diols are optically active and the EH are stereoselective (e.g., enantioselective).

During biotransformation, the substrate (e.g., epoxide) may be metered out continuously or in batch mode to the reaction mixture. Where the epoxide substrates are optically active, the process can use an initial total racemic epoxide concentrations (including two phase systems) from 0.01 M to 5 M or with continuous feeding of epoxide to reach an equivalent epoxide or diol concentration within this range.

Similarly, a biocatalyst exhibiting stereoselective (e.g., enantioselective) EH activity can be added batchwise or continuously during the reaction to maintain necessary activity in order to reach completion. In one embodiment, for example, whole cells of recombinant Yarrowia (e.g., Y. lipolytica) exhibiting stereoselective epoxide hydrolase activity can be added into the initial batch mixture.

A process for stereoselective (e.g., enantioselective) hydrolysis of a racemic epoxide using an epoxide hydrolase biocatalyst expressed in or produced by a recombinant Yarrowia (e.g., Y. lipolytica) strain may be carried out at a pH between 5 and 10 (e.g., between 6.5 and 9, or between 7 and 8.5).

The temperature can be between 0° C. and 60° C. (e.g., between 0° C. and 40° C., or between 0 and 20° C.). Lowering of the reaction temperature can enhance the enantioselectivity of an EH polypeptide.

The amount of biocatalyst in accordance with the present invention added to the reaction containing substrate (e.g., epoxide) in aqueous matrix and biocatalyst in the form of whole cells, cell extracts, crude or purified enzyme preparations that can be free or immobilised, depends on the kinetic parameters of the specific reaction and the amount of epoxide substrate that is to be hydrolysed. In the case of product inhibition negatively affecting the progress of a biocatalytic resolution of racemic epoxide, it may also be advantageous to remove the formed product (i.e., diol) from the reaction mixture or to maintain the concentration of the product at levels that allow reasonable reaction rates.

A reaction mixture containing the recombinant stereoselective epoxide hydrolase biocatalyst may comprise, for example, water, mixtures of water with one or more water miscible organic solvents. Solvents may be added to such a concentration that the polypeptide derived from yeast having activity (e.g., epoxide hydrolase activity) in the formulation used retain hydrolytic activity that is measurable. Examples of water-miscible solvents that may be used include, without limitation, acetone, methanol, ethanol, propanol, isopropanol, acetonitrile, dimethylsulfoxide, N,N-dimethylformamide and N-methylpyrrolidine and the like. However, it is desirous that these solvents be minimised and preferably excluded in the biocatalytic reaction mix.

A biotransformation reaction mixture may also comprise, for example, two-phase systems comprising water and one or more water immiscible solvents. Examples of water immiscible solvents that may be used include, without limitation, toluene, 1,1,2-trichlorotrifluoroethane, methyl tert-butyl ether, methyl isobutyl ketone, dibutyl-o-phthalate, aliphatic alcohols containing 6 to 10 carbon atoms (e.g., hexanol, octanol, decanol), aliphatic hydrocarbons containing 6 to 16 carbon atoms (for example cyclohexane, n-hexane, n-octane, n-decane, n-dodecane, n-tetradecane and n-hexadecane or mixtures of the aforementioned hydrocarbons) and the like. However, use of such solvents typically is minimized, and may be excluded from the biocatalytic reaction mix altogether.

In addition, a buffer may be added to a biotransformation reaction mixture to maintain pH stability. For example, 0.05 M phosphate buffer pH 7.5 may be suitable for most applications in the case of chiral epoxide resolution.

The progress of biotransformation may be monitored using standard procedures such as those known in the art, which include, for example, gas chromatography or high-performance liquid chromatography on columns containing non-chiral or chiral stationary phases.

In the case of stereoselective (e.g., enantioselective) resolution of racemic epoxides, the reaction can be stopped when one enantiomer of the epoxide and/or vicinal diol is found to be at the target enantiomeric excess compared to the other enantiomer of the epoxide and/or vicinal diol. In one embodiment, the reaction is stopped when one enantiomer of the epoxide and/or associated vicinal diol product is found to be in an enantiomeric or diastereomeric excess of at least 75%. In another embodiment, the reaction is stopped when either the diol product or the unreacted epoxide substrate is present at >95% enantiomeric excess, or even at substantially 100% enantiomeric excess (practically measured at ≧98% ee).

A reaction may be stopped by, for example, separation of the biocatalyst (i.e., preparations of recombinant Yarrowia cells containing a polypeptide of microbial, insect, plant and animal (mammalian and invertebrate) origin having biocatalytic activity such as whole cells, cell extracts or crude or purified enzyme extracts, which can be free or immobilized) from the reaction mixture using techniques known to those of skill in the art (e.g., centrifugation, membrane filtration and the like) or by temporary or permanent inactivation of the catalyst (for example by extreme temperature exposure or addition of salts and/or organic solvents).

Residual substrates and products (e.g., optically active epoxides and/or vicinal diols) produced by the biotransformation reaction may be recovered from the reaction medium, directly or after removal of the biocatalyst, using methods such as those known in the art, e.g., extraction with an organic solvent (such as hexane, toluene, diethyl ether, petroleum ether, dichloromethane, chloroform, ethyl acetate and the like), vacuum concentration, crystallization, distillation, membrane separation, column chromatography and the like.

Methods and materials are described below in examples which are meant to illustrate, not limit, the invention. Skilled artisans will recognize methods and materials that are similar or equivalent to those described herein, and that can be used in the practice or testing of the present invention.

### EXAMPLES

### Example 1

### Cloning of EH Coding Sequences from Diverse Origins into Expression Vectors and Production of Y. lipolytica Recombinant Strains

Selection of Representative Epoxide Hydrolases from the Full Spectrum of Available Epoxide Hydrolase Classes and Families.

Barth et al. (2004) performed systematic analyses on the sequences and structures of all known and putative EH obtained from the NCBI (National Center for Biotechnology Information, Bethesda, Md.) GenBank database. The search delivered 95 EH, including 56 putative EH. Subsequent multiple alignments and phylogenetic analysis separated these EH in microsomal (mEH) and cytosolic (sEH) families. The mEH family could be subdivided into 4 main homologous EH families of mammalian, insect, bacterial and fungal origin (FIG. 2). Representative examples of EH encoding genes were selected from the different subdivisions of mEH to span the entire range. In addition, sEH were selected from plant and bacterial origin to give a selection that would be representative of both the mEH and sEH families.

TABLE 1
List of microsomal and cytosolic EH used to demonstrate the generic
applicability of Yarrowia lipolytica as a expression system for the
functional expression of epoxide hydrolases from diverse sources

| | | |
|---|---|---|
| | | GenBank/EMBL |
| Coding sequence origin | NCBI accession No. | accession no. |

Microsomal EH

| | | |
|---|---|---|
| Trichoplasia ni | AAB88192 | |
| Trichoplasia ni | AAB18243 | |
| Homo sapiens | A2993 | |
| Aspergillus niger | CAB59813 | AJ238460 |
| Aspergillus. Niger | AAX78198 | AY966486 |
| Cryptooccus neoformans | DAA02300 | |
| Rhodotorula mucilaginosa (#23) | AAV64029 | |
| Rhodosporidium toruloides | AAF64646 | |
| (#46) | | |
| Rhodotorula araucariae (#25) | AAN32663 | |
| Rhodosporidium paludigenum | AAO72994 | |
| (#692) | | |

Cytosolic (soluble) EH

| | | |
|---|---|---|
| Agrobacterium radiobacter AD1 | ARECHA Y12804 | |
| Solanum tuberosum | STU02497 | |
| Candida albicans | XP_719692 | EAL00941 |

Conceptual translation of all the above-listed EH coding sequences, followed by amino acid homology analysis, indicated sequence homology levels ranging from 14%-73% at the amino acid level (FIG. 3).

### Microbial Strains, Plasmids and Oligonucleotides

All microbial strains, plasmids, and oligonucleotides used in this study are listed in Tables 2, 3 and 4, respectively.

TABLE 2
Microbial strains used in Example 1

| | | Source/ |
|---|---|---|
| Strain | Genotype/Description | Reference |
| Y. lipolytica Po1g | MATA, leu2-270, ura3-302::URA3, xpr2-322, | Madzak et al. |
| | axp-2, XPR2^{p}::SUC2. | (2000) |
| E. coli XL-10 Gold | Tet^{r} D(mcrA)183 D(mcrCB-hsdSMR-mrr)173 | Stratagene, |
| | endA1 supE44 thi-1 recA1 gyrA96 relA1 lac Hte | USA |
| | [F′ proAB lacI^{q}ZDM15 Tn10 (Tet^{r}) Amy Cam^{r}]. | |
| A. niger CBS | | Gordon et al., |
| 120.49 | | 2000 |
| C. neoformans | #777 | CBS 132 |
| R. mucilaginosa | #23 | UOFS Y-0137 |
| R. araucariae | #25 | NCYC 3183 |
| R. toruloides | #46 | UOFS Y-0471 |
| R. toruloides | #1 | NCYC 3181 |
| R. paludigenum | #692 | NCYC 3179 |
| C. albicans | | UOFS Y-0198 |
| YL-sTsA-Tn1 | Po1g transformed with pKOV136 carrying the | This study |
| | mEH 1 (U73680) from T. ni | |
| YL-sTsA-Tn2 | Po1g transformed with pKOV136 carrying the | This study |
| | gut mEH 2 (AF035482) from T. ni | |
| YL-sTsA-Hs | Po1g transformed with pKOV136 carrying the | This study |
| | mEH from H. sapiens | |
| YL-sTsA-An1 | Po1g transformed with pKOV136 carrying the | This study |
| | mEH AJ from A. niger | |
| YL-sTsA-An2 | Po1g transformed with pKOV136 carrying the | This study |
| | mEH AY from A. niger | |
| YL-777 sTsA | Po1g transformed with pKOV136 carrying the | This study |
| | mEH from C. neoformans (CBS 132) #777. | |
| YL-23 sTsA | Po1g transformed with pKOV136 carrying the | This study |
| | mEH from R. mucilaginosa (UOFS Y-0198) #23. | |
| YL-25 sTsA | Po1g transformed with pKOV136 carrying the | This study |
| | mEH from R. araucariae (NCYC 3183) #25. | |
| YL-46 sTsA | Po1g transformed with pKOV136 carrying the | This study |
| | mEH from R. toruloides (UOFS Y-0471) #46. | |
| YL-692 sTsA | Po1g transformed with pKOV136 carrying the | This study |
| | mEH from R. paludigenum (NCYC 3179) #692. | |
| YL-sTsA-Ar | Po1g transformed with pKOV136 carrying the | This study |
| | sEH from A. radiobacter | |
| YL-sTSA-St | Po1g transformed with pKOV136 carrying the | This study |
| | sEH from S. tuberosum | |
| YL-sTSA-Ca | Po1g transformed with pKOV136 carrying the | This study |
| | sEH from C. albicans (UOFS Y-0198). | |
| Y. lipolytica Po1h | MATA, ura3-302, uxpr2-322, axp1-2) | Madzak et al. |
| | | (2003) |
| YL-Tn1-HmA | Po1h transformed with pYLHmA carrying the | This study |
| | mEH 1 (U73680) from T. ni | |
| YL-Tn2-HmA | Po1h transformed with pYLHmA carrying the | This study |
| | gut mEH 2 (AF035482) from T. ni | |
| YL-Hs-HmA | Po1h transformed with pYLHmA carrying the | This study |
| | mEH from H. sapiens | |
| YL-An1-HmA | Po1h transformed with pYLHmA carrying the | This study |
| | mEH AJ from A. niger | |
| YL-An2-HmA | Po1h transformed with pYLHmA carrying the | This study |
| | mEH AY from A. niger | |
| YL-23 HmA | Po1h transformed with pYLHmA carrying the | This study |
| | mEH from R. mucilaginosa (UOFS Y-0198). | |
| YL-777 HmA | Po1h transformed with pYLHmA carrying the | This study |
| | mEH from C. neoformans (CBS 132). | |
| YL-25 HmA | Po1h transformed with pYLHmA carrying the | This study |
| | mEH from R. araucariae (NCYC 3183). | |
| YL-46 HmA | Po1h transformed with pYLHmA carrying the | This study |
| | mEH from R. toruloides (UOFS Y-0471 | |
| YL-1 HmA | Po1h transformed with pYLHmA carrying the | This study |
| | mEH from R. toruloides (NCYC 3181) | |
| YL-692 HmA | Po1h transformed with pYLHmA carrying the | This study |
| | mEH from R. paludigenum (NCYC 3179). | |
| YL-Ar-HmA | Po1h transformed with pYLHmA carrying the | This study |
| | sEH from A. radiobacter | |
| YL-St-HmA | Po1h transformed with pYLHmA carrying the | This study |
| | sEH from S. tuberosum | |
| YL-Ca-HmA | Po1h transformed with pYLHmA carrying the | This study |
| | sEH from C. albicans (UOFS Y-0198). | |

TABLE 3
Plasmids used in Example 1

| | | Source/ |
|---|---|---|
| Plasmid | Description | Reference |
| pGEM ®-T | General vector containing T overhangs for cloning of | Promega, |
| Easy | adenylated PCR products. | USA |
| pPCR-Script | General cloning vector | Stratagene, |
| | | USA |
| pINA781 | pBR322 based integrative vector for site directed | Madzak et |
| | integration at the pBR322 docking site (integration- | al., 1999 |
| | target sequence) in the genome of Po1g. | |
| pINA1313 | Single copy integrative shuttle vector containing Kan^{R} | Nicaud et al. |
| | and ura3d1 selective markers. Random integration into | (2002) |
| | Po1h genome through the ZETA transposable element. | |
| | The plasmid contains the synthetic promoter, hp4d, and | |
| | the Y. lipolytica LIP2 signal peptide. | |
| pKOV96 | Zeta element based integrative vector carrying the non- | This study |
| | defective ura3d1 selection marker. Similar to | |
| | pINA1313, with hp4d replaced with TEF promoter and | |
| | Y. lipolytica LIP2 signal sequence removed. | |
| pKOV136 | pINA781 with the β-galactosidase gene replaced by the | This study |
| | promoter-MCS-terminator region from pKOV96. | |
| pGEM-Hs | pGEM ®-T Easy harboring the mEH ORF from H. sapiens. | This study |
| pcrSMART- | pcrSMART ™ harboring the mEH AJ ORF from A. niger. | This study |
| An1 | | |
| pcrSMART- | pcrSMART ™ harboring the mEH AY ORF from A. niger. | This study |
| An2 | | |
| pGEM-777 | pGEM ®-T Easy harboring the EH ORF from C. neoformans | This study |
| | (CBS 132). | |
| pGEM-23 | pGEM ®-T Easy harboring the mEH ORF from R. mucilaginosa | This study |
| | (UOFS Y-0198). | |
| pGEM-46 | pGEM ®-T Easy harboring the mEH ORF from R. toruloides | This study |
| | (UOFS Y-0471). | |
| pGEM-25 | pGEM ®-T Easy harboring the mEH ORF from R. araucariae | This study |
| | (NCYC 3183). | |
| pGEM-692 | pGEM ®-T Easy harboring the mEH ORF from R. paludigenum | This study |
| | (NCYC 3179). | |
| pGEM-Ar | pGEM ®-T Easy harboring the EH ORF from A. radiobacter. | This study |
| pPCR- | pPCR-Script harboring the soluble EH ORF from | This study |
| Script-St | S. tuberosum | |
| pGEM-Ca | pGEM ®-T Easy harboring the sEH ORF from C. albicans | This study |
| | (UOFS Y-0198). | |
| pKOV136- | pKOV136 harboring the microsomal EH 1 (U73680) | This study |
| Tn1 | ORF from T. ni. | |
| pKOV136- | pKOV136 harboring the gut microsoinal EH 2 | This study |
| Tn2 | (AF035482) ORF from T. ni. | |
| pKOV136- | pKOV136 harboring the microsomal EH ORF from H. sapiens. | This study |
| Hs | | |
| pKOV136- | pKOV136 harboring the soluble EH AJ ORF from A. niger. | This study |
| An1 | | |
| pKOV136- | pKOV136 harboring the soluble EH AY ORF from A. niger. | This study |
| An2 | | |
| pKOV136- | pKOV136 harboring the EH ORF from C. neoformans | This study |
| 777 | (CBS 132). | |
| pKOV136- | pKOV136 harboring the EH ORF from R. mucilaginosa | This study |
| 23 | (UOFS Y-0198). | |
| pKOV136- | pKOV136 harboring the EH ORF from R. toruloides | This study |
| 46 | (UOFS Y-0471). | |
| pKOV136- | pKOV136 harboring the EH ORF from R. araucariae | This study |
| 25 | (NCYC 3183). | |
| pKOV136- | pKOV136 harboring the EH ORF from R. paludigenum | This study |
| 692 | (NCYC 3179). | |
| pKOV136- | pKOV136 harboring the soluble EH ORF from A. radiobacter. | This study |
| Ar | | |
| pKOV136- | pKOV136 harboring the soluble EH ORF from S. tuberosum | This study |
| St | | |
| pKOV136- | pKOV136 harboring the EH ORF from C. albicans | This study |
| Ca | (UOFS Y-0198). | |
| pYLHmA = pINA1291 | Multiple copy integrative shuttle vector containing Kan^{R} | Nicaud et al |
| | and ura3d4 selective markers. Random integration into | (2002) |
| | Po1h genome through the ZETA transposable element. | |
| | The plasmid contains the synthetic promoter, hp4d. | |
| pYL-Tn1- | pYLHmA harboring the microsomal EH 1 (U73680) | This study |
| HmA | ORF from T. ni. | |
| pYL-Tn2- | pYLHmA harboring the gut microsomal EH 2 | This study |
| HmA | (AF035482) ORF from T. ni. | |
| pYL-Hs- | pYLHmA harboring the microsomal EH ORF from H. sapiens. | This study |
| HmA | | |
| pYL-An1- | pYLHmA harboring the soluble EH AJ ORF from A. niger. | This study |
| HmA | | |
| pYL-An2- | pYLHmA harboring the soluble EH AY ORF from A. niger. | This study |
| HmA | | |
| pYL-777- | pYLHmA harboring the EH ORF from C. neoformans | This study |
| HmA | (CBS 132). | |
| pYL-23- | pYLHmA harboring the EH ORF from R. mucilaginosa | This study |
| HmA | (UOFS Y-0198). | |
| pYL-25- | pYLHmA harboring the mEH ORF from R. araucariae | This study |
| HmA | (NCYC 3183). | |
| pYL-46 | pYLHmA harboring the EH ORF from R. toruloides | This study |
| HmA | (UOFS Y-0471). | |
| pYL-1- | pYLHmA harboring the EH ORF from R. toruloides | This study |
| HmA | (NCYC 3181). | |
| pYL-692- | pYLHmA harboring the EH ORF from R. paludigenum | This study |
| HmA | (NCYC 3179). | |
| pYL-Ar- | pYLHmA harboring the soluble EH ORF from | This study |
| HmA | A. radiobacter. | |
| pYL-St- | pYLHmA harboring the soluble EH ORF from | This study |
| HmA | S. tuberosum | |
| pYL-Ca- | pYLHmA harboring the EH ORF from C. albicans | This study |
| HmA | (UOFS Y-0198). | |

TABLE 4
Oligonucleotide primers used in Example 1

| | | Restriction |
|---|---|---|
| | | sites |
| Primer Name | Sequence in 5′ to 3′orientation | Introduced |
| T. ni 1-1F | GGATCCATGGGTCGCCTCTTATTCCTAGTGC | BamHI |
| | (SEQ ID NO:1) | |
| T. ni 1-1R | GCCTAGGTCACAAATCAGTCTTCTCGTTATTCTTCTGTAGC | AvrII |
| | (SEQ ID NO:2) | |
| T. ni 2-1F | GAGATCTATGGCCCGTCTCCTCTTCATACTACCAG | BglII |
| | (SEQ ID NO:3) | |
| T. ni 2-1F | GCCTAGGTTACAAATCAGTCTTGACATTCTTCTTCTGCAG | AvrII |
| | (SEQ ID NO:4) | |
| H. sap mEH-1F | GGATCCATGTGGCTAGAAATCCTCCTCACTTCAGTGC | BamHI |
| | (SEQ ID NO:5) | |
| H. sap mEH-1R | GCCTAGGTCATTGCCGCTCCAGCACC | AvrII |
| | (SEQ ID NO:6) | |
| A. niger AJ-1F | GGATCCATGTCCGCTCCGTTCGCCAAG | BamHI |
| | (SEQ ID NO:7) | |
| A. niger AJ-1R | CCTAGGCTACTTCTGCCACACCTGCTCGACAAATG | AvrII |
| | (SEQ ID NO:8) | |
| A. niger AY-1F | GGATCCATGGCACTCGCTTACAGCAACATTCCC | BamHI |
| | (SEQ ID NO:9) | |
| A. niger AY-1R | CCTAGGTCATTTTCTACCAGCCCATACTTGTTCACAGAACGC | AvrII |
| | (SEQ ID NO:10) | |
| C. neoformans- | TGG ATC CAT GTC GTA TTC AGA CCT TCC CC | BamHI |
| 1F | (SEQ ID NO:11) | |
| C. neoformans- | TGC TAG CTC AGT AAT TAC CTT TGT ACT TCT CCC AC | NheI |
| 1R | (SEQ ID NO:12) | |
| R. mucilaginosa- | AGA TCT ATG CCC GCC CGC TCG CTC | BglII |
| 1F | (SEQ ID NO:13) | |
| R. mucilaginosa- | TCC TAG GCT ACG ATT TTT GCT CCT GAG AGA GAG | AvrII |
| 1R | (SEQ ID NO:14) | |
| R. toruloides-1F | GTGGATCCATGGCGACACACA | BamHI |
| | (SEQ ID NO:15) | |
| R. toruioides-1R | GACCTAGGCTACTTCTCCCACA | AvrII/BlnI |
| | (SEQ ID NO:16) | |
| R. araucariae-1F | GATTAATGATCAATGAGCGAGCA | BclI |
| | (SEQ ID NO:17) | |
| R. araucariae-1R | GACCTAGGTCACGACGACAG | BlnI |
| | (SEQ ID NO:18) | |
| R. paludigenum- | GTGGATCCATGGCTGCCCA | BamHI |
| 1F | (SEQ ID NO:19) | |
| R. paludigenum- | GAGCTAGCTCAGGCCTGG | NheI |
| 1R | (SEQ ID NO:20) | |
| A. radiobacter- | GGGATCCATGGCAATTCGACGTCCAGAAGAC | BamHI |
| 2F | (SEQ ID NO:21) | |
| A. radiobacter- | GCCTAGGCTAGCGGAAAGCGGTCTTTATTCG | AvrII |
| 2R | (SEQ ID NO:22) | |
| S. tuberosum-1F | GAGGATCCATGGAGAAGATAG | BamHI |
| | (SEQ ID NO:25) | |
| S. tuberosum-1R | GACCTAGGTTAAAACTTTTGATAG | AvrII |
| | (SEQ ID NO:26) | |
| C. albicans-1F | GGG ATC CAT GAC AAA ATT TGA TAT CAA G | BamHI |
| | (SEQ ID NO:27) | |
| C. albicans-1R | GCC TAG GTT ATT TAG AAT ATT TTT CGA AAA AAT C | AvrII |
| | (SEQ ID NO:28) | |
| Integration-1F | CCTAGGGTGTCTGTGGTATCTAAGC Integration screening for | |
| | (SEQ ID NO:29) | Po1g |
| Integration-1R | CCGTCTCCGGGAGCTGC | Integration screening for |
| | (SEQ ID NO:30) | Po1g |
| pINA-1 | CATACAACCACACACATCCA | Integration screening for |
| | (SEQ ID NO:31) | Po1h |
| pINA-2 | TAAATAGCTTAGATACCACAG | Integration screening for |
| | (SEQ ID NO:32) | Po1h |

Underlining indicates the sequences of introduced restriction sites.

Construction of pKOV136, a Constitutive, Site-Specific, Single Copy Integrative Vector (sTSA Transfomants).

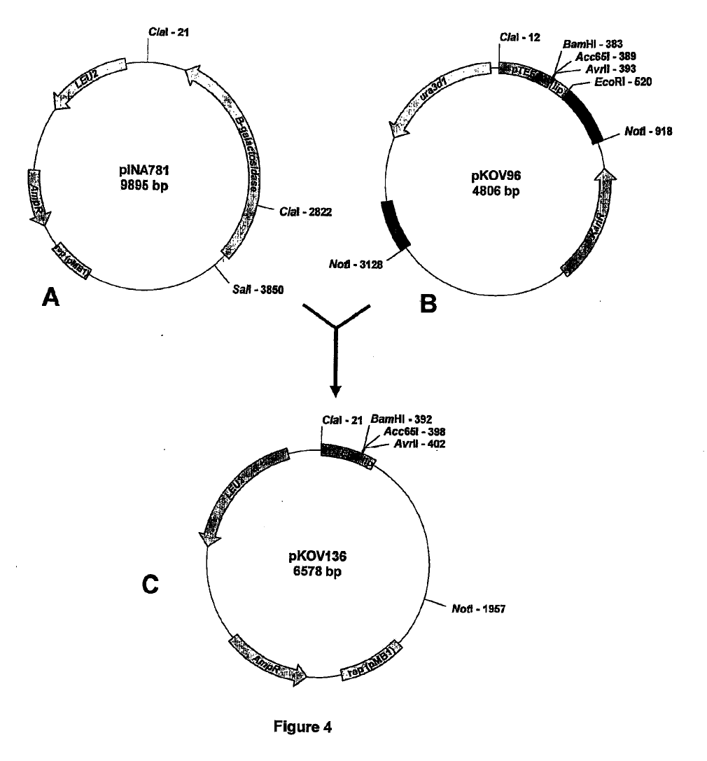

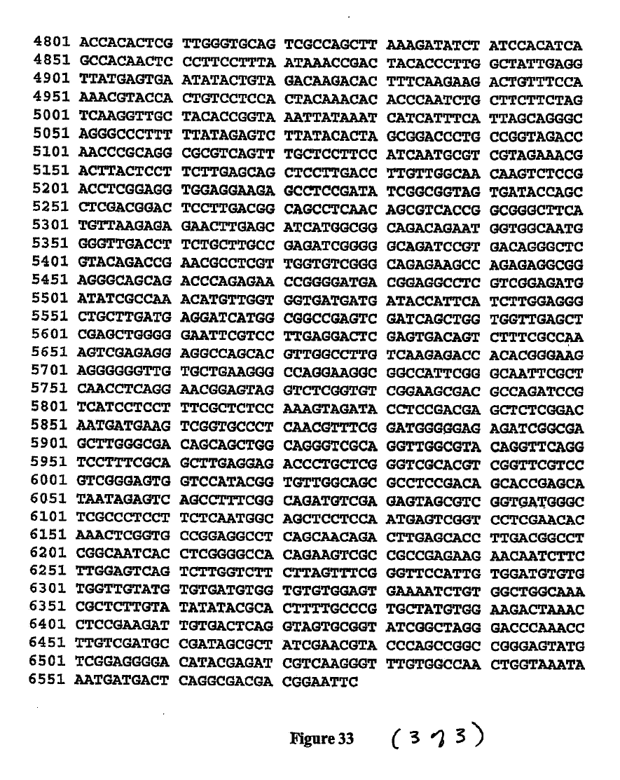

The pKOV136 vector (FIG. 4) was designed to overcome the problems of inconsistent copy number and random integration in the genome of strains devoid of the Ylt1 retrotransposon (Pignède et al., 2000). The pKOV136 vector is based on the pINA781 vector, which in turn is based on the pBR322 backbone (Madzak et al., 1999). The pBR322-based vector allows for site directed, single crossover, homologous recombination and integration at the pBR322 docking site (integration-target sequence; a region introduced into the Po1g genome that contains part of the E. coli cloning vector, pBR322, to afford homologous recombination upon transformation of pKOV136) in the genome of Y. lipolytica Po1g, thereby allowing expression cassettes to be exposed to the same level of transcriptional accessibility (see FIG. 33). The homologous recombination allows for 80% of expression cassettes to be integrated at the correct site (Barth and Gaillardin, 1996).

By combining the beneficial properties of the TEF promoter (constitutive expression that eliminates possible induction differences and allows for fast and efficient screening of transformants) from pKOV96 with the site specific integration targeting of the pBR322 docking system from pINA781, it is possible to obtain the ideal expression system for comparative studies. The system not only allows site specific integration, but due to the homologous single crossover recombination that occurs at the pBR322 docking site in the Po1g genome, it also increases transformation efficiency compared to non-homologous systems (Pignède et al., 2000).

The pKOV96 and pINA781 vectors were first digested with EcoRI and SalI, respectively, followed by filling of the 3′ recessed ends using Klenow DNA polymerase to create blunt-ended molecules. Both sets of vectors were subsequently treated with ClaI allowing the liberation of the TEF promoter, multiple cloning site and LIP2 terminator from pKOV96 and the region containing the β-galactosidase coding sequence from pINA781.

The TEF promoter, multiple cloning site and LIP2 terminator fragment was inserted into the compatible pINA781 backbone, resulting in plasmid pKOV136 (FIG. 4). The nucleotide sequence (SEQ ID NO:24) of pKOV136 is shown in FIG. 33.

The PKOV136 vector was deposited under the Budapest Treaty on ______ at the European Collection of Cell Culture (ECACC), Health Protection Agency, Porton Down, Salisbury, Wiltshire, SP4 OJG and is identified by the ECACC accession number ______. The sample deposited with the ECACC was taken from the same deposit maintained by the Oxyrane (Pty, Ltd.) since prior to the filing date of this application. The deposit will be maintained without restriction in the ECACC depository for a period of 30 years, or 5 years after the most recent request, or for the effective life of the patent, whichever is longer, and will be replaced if the deposit becomes non-viable during that period.

The pGEM®-T Easy, pGEM7f and pcrSmart vectors harboring the EH encoding coding sequences from the various sources as well as pKOV136 plasmids, were digested with the appropriate restriction enzymes to create compatible cohesive ends suitable for ligation of the EH into the BamHI-AvrII cloning sites of the pKOV136 plasmids.

The EH encoding coding sequences from the various sources were cloned into the pKOV136 vector and used to transform the Po1g recipient strain.

pYLHmA, a Multi-Copy Integrative Vector Without a Secretion Signal (HmA Transformants)

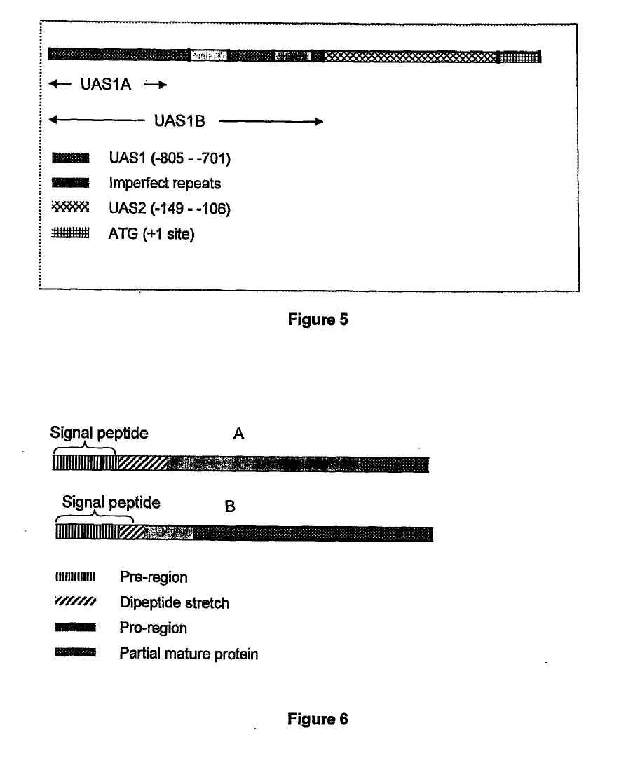

The pINA1291 vector (FIG. 5) was obtained from Dr. Catherine Madzak of labo de Génétique, INRA, CNRS, France. This vector was renamed pYLHmA (Yarrowia Lipolytica expression vector, with Hpd4 promoter, Multi-copy integration selection, A=no secretion signal)

### Nucleic Acid Isolation, Amplification, Cloning and Sequencing of Epoxide Hydrolase Coding Sequences.

The EH coding sequences from Solanum tuberosum were synthesized by GeneArt GmbH, Regeneburg, Germany. The Trichoplasia ni EH coding sequence was obtained from North Carolina State University, North Carolina. U.S.A. The S. tuberosum (St) coding sequence was recoded for Y. lipolytica codon bias. The synthetic coding sequences were received as fragments cloned into pPCR-Script (Stratagene, La Jolla, Calif., U.S.A). The S. tuberosum and T. ni1 coding sequence were obtained with flanking BamHI and AvrII recognition sites. The T. ni 2 sequence was flanked by BglII and AvrII.

Yeast strains (Cryptooccus neoformans (CBS 192), Rhodotorula mucilaginosa (UOFS Y-0137), Rhodosporidium toruloides (UOFS Y-0471), Rhodotorula araucariae (UOFS Y-0473) and Candida albicans (UOFS Y-0198)) were obtained from the UOFS (University of the Orange Free State, Bloemfontein, Republic of South Africa) yeast culture collection and were cultivated in 50 ml YPD media (20 g/l peptone; 20 g/l glucose; 10 g/l yeast extract) at 30° C. for 48 hours while shaking. Cells were harvested by centrifugation and the resulting pellet was either frozen at −70° C. for RNA isolation or suspended to a final concentration of 20% (w/v) in 50 mM phosphate buffer (pH 7.5) containing 20% (v/v) glycerol and frozen at −70° C. for DNA isolation. Aspergillus niger (CBS 120.49) was cultivated as described by Arand et al., 1999.

DNA isolation entailed addition of 500 μl lysis solution (100 mM Tris-HCl, pH 8.0; 50 mM EDTA, pH 8.0; 1% SDS) and 200 μl glass beads (425-600 μm diameter) to 0.4 g wet cells, followed by vortexing for 4 min, cooling on ice and addition of 275 μl ammonium acetate (7 M, pH 7.0). After incubation at 65° C. for 5 min followed by 5 min on ice, 500 μl chloroform was added, vortexed and centrifuged (20 000×g, 2 min, 4° C.). The supernatant was transferred and the DNA precipitated for 5 min at room temperature using 1 volume iso-propanol and centrifuged (20 000×g, 5 min, 4° C.). The pellet was washed with 70% (v/v) ethanol, dried and re-dissolved in 100 μl TE (10 mM Tris-HCl; 1 mM EDTA, pH 8.0).

Total RNA isolation entailed grinding 10 g wet cells under liquid nitrogen to a fine powder, 0.5 ml of the powder was added to a pre-cooled 1.5 ml polypropylene tube and thawed by the addition of TRIzol® solution (InVitrogen, Carlsbad, Calif., U.S.A.). The isolation of total RNA using TRIzol® was performed according to the manufacturer's instructions. The total RNA isolated was suspended in 50 μl formamide and frozen at −70° C. for further use. Total RNA was similarly isolated from Aspergillus niger (CBS 120.49).

Reverse transcription of total RNA into cDNA was peformed as follows. Oligonucleotide primers were designed according to the sequence data available and used in a two step RT-PCR reaction. First strand cDNA synthesis was performed on total RNA using Expand Reverse Transcriptase (Roche Applied Science, Indianapolis, Ind., U.S.A.) in combination with primer Rm cDNA-2R at 42° C. for 1 hour followed by heat inactivation for 2 minutes at 95° C. The newly synthesized cDNA was amplified using primers Rm cDNA-2F and Rm cDNA-1R (Table 4) (initial denaturation for 2 minutes at 94° C.; followed by 30 cycles of 94° C. for 30 sec; 67° C. for 30 sec; 72° C. for 2 min and a final elongation of 72° C. for 7 min).

Forward and reverse primers (Table 4) were designed to introduce the required restriction sites during PCR to allow for subcloning of the EH encoding coding sequences into the single-copy vector pKOV136 or the multi-copy vector pYL-HmA. All non-synthetic EH encoding coding sequences, except for the A. niger coding sequences, were PCR amplified using Expand High Fidelity Plus PCR System (Roche Applied Sciences). Thermal cycling entailed initial denaturation of 2 min at 94° C. followed by 30 cycles of 94° C. for 30 sec, Tₘ-5° C. for 30 sec (Tₘ was calculated using the modified nearest neighbor calculation obtained from Integrated DNA Technologies, Coralville, Iowa, U.S.A; www.idtdna.com) and 72° C. for 2 min. A 72° C. (10 min) final elongation step was included to allow complete synthesis of amplified DNA. PCR products were electrophoretic gel purified and cloned into pGEM®-T Easy.

The EH encoding coding sequences from A. niger were PCR amplified using Phusion™ DNA polymerase (Finnzymes, Espoo, Finland) during thermal cycling that entailed initial denaturation of 30 sec at 98° C., followed by 30 cycles of 98° C. for 10 sec and 72° C. for 45 sec. The 2-step amplification was followed by a final elongation of 10 min at 72° C. The PCR products were cloned into pcrSMART™ vector using the PCR-SMART™ cloning kit

The synthesized coding sequences from Solanum tuberosum was received as a fragment cloned into pPCR-Script (Stratagene).

Vectors containing the EH encoding coding sequences of interest were transformed into XL-10 Gold® E. coli for plasmid amplification and sequencing. The EH encoding coding sequences were subjected to restriction and sequence analysis before transfer of the coding sequences from the cloning vectors to the expression vectors.

The cloning vectors containing the EH encoding coding sequences were treated with the restriction enzyme pairs indicated in Table 4 to liberate the EH encoding coding sequences.

The liberated fragments were ligated into BamHI and AvrII linearized pKOV136 or pYLHmA expression vectors.

### Transformation, Activity Screening and Selection of YL-sTSA Transformants

Y. lipolytica Po1g cells were transformed with NotI linearized pKOV136 vector containing the EH encoding coding sequences (according to the method described by Xuan et al., 1988) and plated onto YNB_{N5000} plates [YNB without amino acids and ammonium sulfate (1.7 g/l), ammonium sulfate (5 g/l), glucose (10 g/l) and agar (15 g/l)].

Viable transformants were subjected to qualitative activity screening by thin layer chromatography (TLC). Transformants exhibiting EH activity were subjected to genomic DNA isolation, followed by PCR screening to confirm integration at the pBR322 docking site (integration-target sequence). PCR screening of Po1g transformants for correct integration at the pBR322 docking site (integration-target sequence) entailed amplification of a ˜1.6 kb fragment using primers Integration-1F and Integration-1R in a standard PCR (annealing at 56° C.). Copy number was confirmed using the isolated genomic DNA from positive transformants (exhibiting the correct PCR product). DNA was digested with ApaI and subjected to hybridization with the leu2 DIG-labeled probe.

Po1g transformants that tested positive for activity, copy number and integration site were inoculated into 200 ml YPD and incubated while shaking at 28° C. for 48 hours. Cells were harvested by centrifugation (6 000×g for 5 min), washed with and resuspended in 50 mM phosphate buffer (pH 7.5) containing 20% glycerol (v/v) to a final concentration of 50% (w/v) and stored at −20° C. for future experiments.

### Transformation and Selection of Multiple Copy Transformants (YL-HmA Transformants)

Y. lipolytica Po1h cells were transformed with NotI linearized pYL-HmA vector containing the EH encoding coding sequences (according to the method described by Xuan et al., 1988) and plated onto YNB_{casa} plates [YNB without amino acids and ammonium sulfate (1.7 g/l), ammonium chloride (4 g/l), glucose (20 g/l), casamino acids (2 g/l), and agar (15 g/l)].

Transformants were subjected to genomic DNA isolation, followed by PCR screening to confirm presence of the integrated NotI-expression cassette. This entailed amplification of a ˜1.6 kb fragment using primers pINA-1 and pINA-2 in a standard PCR (annealing at 50° C.).

Po1 h transformants that tested positive for activity were inoculated into 200 ml YPD and incubated while shaking at 28° C. for 48 hours (stationary phase). Cells were harvested by centrifugation (6 000×g for 5 min), washed with and resuspended in 50 mM phosphate buffer (pH 7.5) containing 20% glycerol (v/v) to a final concentration of 50% (w/v) and stored at −20° C. for future experiments.

### Example 2

### Functional Expression of Fungal Epoxide Hydrolases in Yarrowia lipolytica

1. Construction of Single Copy (pMic62) and Multicopy (pMic64) Plasmids Containing the Inducible XPR2^{p} Promoter and (a) the Native Y. lipolytica XPR2^{p} Prepro-Region as Signal Peptide and (b) the Trichoderma reesii Signal Peptide

### Microbial Strains, Plasmids, and Oligonucleotide Primers

All of the microbial strains, plasmids and oligonucleotide primers used during this study are listed in Tables 5, 6 and 7 respectively.

TABLE 5
Microbial strains used in Example 2

| | | Source/ |
|---|---|---|
| Strains | Genotype/Description | Reference |
| Y. lipolytica Po1h | MATA, ura3-302, uxpr2-322, axp1-2) | Madzak et al. |
| | | (2003) |
| Yarrowia lipolytica | MATA, ura3-302, leu2-270, xpr2-322, | Le Dall et al. |
| Po1d | XPR2^{p}::SUC2 | (1994) |
| E. coli XL-10 Gold | Tet^{r} D(mcrA)183 D(mcrCB-hsdSMR-mrr)173 | Stratagene, |
| | endA1 supE44 thi-1 recA1 gyrA96 relA1 lac Hte | USA |
| | [F′ proAB lacI^{q}ZDM15 Tn10 (Tet^{r}) Amy Cam^{r}]. | |
| Trichoderma reesei | | VVT |
| | | (QM9414) |
| E. coli Top 10 | CaCl₂ competent cells | Invitrogen |
| | | USA |
| R. mucilaginosa | #23 | NCYC 3190 |
| R. araucariae | #25 | NCYC 3183 |
| R. toruloides | #46 | UOFS Y-0471 |
| R. toruloides | #1 | NCYC 3181 |
| R. paludigenum | #692 | NCYC 3179 |
| YL-23 TsA | Po1h transformed with pYLTsA carrying the | This study |
| | mEH from R. mucilaginosa (NCYC 3190). | |
| YL-25 TsA | Po1h transformed with pYLTsA carrying the | This study |
| | mEH from R. araucariae (NCYC 3183). | |
| YL-46 TsA | Po1h transformed with pYLTsA carrying the | This study |
| | mEH from R. toruloides (UOFS Y-0471) | |
| YL-1 TsA | Po1h transformed with pYLTsA carrying the | This study |
| | mEH from R. toruloides (NCYC 3181) | |
| YL-692 TsA | Po1h transformed with pYLTsA carrying the | This study |
| | mEH from R. paludigenum (NCYC 3179). | |
| YL-25 HmL | Po1h transformed with pYLHmL carrying the | This study |
| | mEH from R. araucariae (NCYC 3183). | |
| YL-46 HmL | Po1h transformed with pYLHmL carrying the | This study |
| | mEH from R. toruloides (UOFS Y-0471 | |
| YL-692 HmL | Po1h transformed with pYLHmL carrying the | This study |
| | mEH from R. paludigenum (NCYC 3179). | |
| YL-46 XsTRsigP | Po1h transformed with pMic62-TRsigP carrying | This study |
| | the mEH from R. toruloides (UOFS Y-0471) | |
| YL-46 | Po1h transformed with pMic62-XPR2 pre-pro | This study |
| XsXPRSsigP | carrying the mEH from R. toruloides (UOFS Y- | |
| | 0471) | |

TABLE 6
Plasmids used in Example 2

| | | Source/ |
|---|---|---|
| Plasmids | Relevant characteristics | Reference |
| pGem-T ® Easy | Cloning vector with protruding T overhangs used to sub- | Promega, |
| | clone the PCR products amplified using Taq DNA | USA |
| | polymerase. | |
| JM62 | Single copy integrative shuttle vector containing Kan^{r} | Nicaud et al. |
| | and URA3d1 markers. Target regions are the zeta | (2002) |
| | elements of the retrotransposon. The plasmid contains | |
| | the inducible POX2^{p} and no signal peptide | |
| JM64 | Multi copy integrative shuttle vector containing Kan^{r} and | Nicaud et al. |
| | URA3d4 markers. Target regions are the zeta elements of | (2002) |
| | the retrotransposon. The plasmid contains the inducible | |
| | POX2^{p} and no signal peptide | |
| pMic62 | Single copy integrative shuttle vector containing Kan^{r} | This study |
| | and URA3d1 markers. Target regions are the zeta | |
| | elements of the retrotransposon. The plasmid contains | |
| | the inducible XPR2^{p} and the Trichoderma reesei | |
| | endoglucanase I signal peptide. | |
| pMic64 | Same characteristics as the pMic62 with the defective | This study |
| | URA3d4 as selective marker yielding higher copy | |
| | numbers (10-13 copies/genome). | |
| pMic62TRsigP | Single copy integrative shuttle vector containing Kan^{r} | This study |
| | and URA3d1 markers. Target regions are the zeta | |
| | elements of the retrotransposon. The plasmid contains | |
| | the inducible XPR2^{p} and the Trichoderma reesei | |
| | endoglucanase I signal peptide. | |
| pMic62-prepro | Same characteristics as the pMic62 with the T. reesei | This study |
| | endoglucanase I signal peptide replaced by the XPR2 | |
| | prepro-region. | |
| pINA1293 = pYLHmL | Multi copy integrative shuttle vector containing Kan^{r} and | Nicaud et al. |
| | URA3d4 markers. Target regions are the zeta elements of | (2002) |
| | the retrotransposon. The plasmid contains the synthetic | |
| | promoter, hp4d and the Y. lipolytica LIP2 signal peptide. | |
| pINA1313 | Same characteristics as the pINA1293 with the defective | Nicaud et al. |
| | URA3d1 as selective marker yielding single copy | (2002) |
| | numbers. Single copy integrative shuttle vector | |
| | containing Kan^{R} and ura3d1 selective markers. Random | |
| | integration into Po1h genome through the ZETA | |
| | transposable element. The plasmid contains the synthetic | |
| | promoter, hp4d, and the Y. lipolytica LIP2 signal peptide. | |
| pKOV96 = pYLTsA | Similar to pINA1313, with hp4d replaced with TEF | This study |
| | promoter and Y. lipolytica LIP2 signal sequence | |
| | removed. | |
| pYL-23 TsA | pYLTsA carrying the mEH from R. mucilaginosa | This study |
| | (NCYC 3190). | |
| pYL-25 TsA | pYLTsA carrying the mEH from R. araucariae (NCYC | This study |
| | 3183). | |
| pYL-46 TsA | pYLTsA carrying the mEH from R. toruloides (UOFS Y- | This study |
| | 0471) | |
| pYL-1 TsA | pYLTsA carrying the mEH from R. toruloides (NCYC | This study |
| | 3181) | |
| pYL-692 TsA | pYLTsA carrying the mEH from R. paludigenum | This study |
| | (NCYC 3179). | |
| pYL-25 HmL | pYLHmL carrying the mEH from R. araucariae (NCYC | This study |
| | 3183). | |
| pYL-46 HmL | pYLHmL carrying the mEH from R. toruloides (UOFS | This study |
| | Y-0471 | |
| pYL-692 HmL | pYLHmL carrying the mEH from R. paludigenum | This study |
| | (NCYC 3179). | |
| pYL-46 | pMic62-TRsigP carrying the mEH from R. toruloides | This study |
| XsTRsigP | (UOFS Y-0471) | |
| pYL-46 | pMic62-XPR2 pre-pro carrying the mEH from R. toruloides | This study |
| XsXPRSsigP | (UOFS Y-0471) | |

TABLE 7
Oligonucleotide primers used in Example 2

| | | Restriction sites |
|---|---|---|
| Primer Name | Sequence in 5′ to 3′ orientation | Introduced |
| R. toruloides-1F | GTGGATCCATGGCGAGACAGA | BamHI |
| | (SEQ ID NO:15) | |
| R. toruloides-1R | GACCTAGGCTACTTCTCCCACA | AvrII/BlnI |
| | (SEQ ID NO:16) | |
| R. araucariae-1F | GATTAATGATCAATGAGCGAGGA | BclI |
| | (SEQ ID NO:17) | |
| R. araucariae-1R | GACCTAGGTCACGACGACAG | BlnI |
| | (SEQ ID NO:18) | |
| R. paludigenum-1F | GTGGATCCATGGCTGCCCA | BamHI |
| | (SEQ ID NO:19) | |
| R. paludigenum-1R | GAGCTAGCTCAGGCCTGG | NheI |
| | (SEQ ID NO:20) | |
| XPR2-1F | AATCGATCATCCACCGGCTAGCG | ClaI |
| | (SEQ ID NO:32) | |
| XPR2-1R | AGGATCCTGTTGGATTGGAGGATTGG | BamHI |
| | (SEQ ID NO:33) | |
| TRsigP-1F | AGGATCCATGGCGCCCTCAG | BamHI |
| | (SEQ ID NO:34) | |
| TRsigP-1R | ACCTAGGGGTCTTGGAGGTGTC | BlnI |
| | (SEQ ID NO:35) | |
| XPR2(pre-pro)-1R | TTTAAATCGCTTGGCATTAGAAGAAGCAGG | DraI |
| | (SEQ ID NO:36) | |
| Constr-1F | GAGGGCGTCGACTACGCCG | |
| | (SEQ ID NO:37) | |
| Constr-1R | GTTTAAAGGCGGCGACGAGCCG | DraI |
| | (SEQ ID NO:38) | |
| TEF-1F | ATC GAT AGA GAC CCG GTT GGC GG | ClaI |
| | (SEQ ID NO:39) | |
| TEF-1R | AAG CTT TTC GGG TGT GAG TTG ACA AGG | HindIII |
| | (SEQ ID NO:40) | |
| -sigP-1F | TCG GAT CCG GTA CCT AGG GTG TCT GTG | BamHI |
| | (SEQ ID NO:41) | |
| -sigP-1R | GAG GAT CCT TCG GGT GTG AGT TGA CAA GGA G | BamHI |
| | (SEQ ID NO:42) | |
| Rm-probe-1F | CTT CGA CTG GGC CAC AAG CTT TTG TC | Hybridization |
| | (SEQ ID NO:43) | probe primer |
| Rm-probe-1R | AGA TTG CGA GGA TCG TGC CGA GG | Hybridization |
| | (SEQ ID NO:44) | probe primer |
| Rm cDNA-2F | AGA TCT ATG CCC GCC CGC TCG CTC | BglII |
| | (SEQ ID NO:45) | |
| Rm cDNA-1R | TCC TAG GCT ACG ATT TTT GCT CCT GAG AGA GAG | AvrII |
| | (SEQ ID NO:46) | |

Underlining indicates the sequence of introduced restriction sites.

Construction of Single- and Multi-Copy Shuttle Vectors Containing the Strongly Inducible XPR2^{p} or the Qusi-Constitutive hp4d^{p} and Different Signal Peptides

Genomic DNA from Y. lipolytica and T. reesei was prepared from 50 ml YPD cultures grown for 5 days at 28° C. The cells were harvested by centrifugation (10 min, 4° C., 5000×g), washed twice with ice cold sterile water and suspended in ice cold sterile water to a final concentration of 20% (w/v). Cell suspensions (3 ml) were aliquoted into 10 ml Pyrex® tubes and centrifuged (10 min, 4° C., 5000×g). The supernatant was discarded and the pellet was suspended in 1 ml DNA lysis buffer [100 mM Tris-HCl (pH 8), 50 mM EDTA, 1% SDS] and kept on ice. One volume of glass beads (200 μm) was added to the suspension and vortexed for 1 minute with immediate cooling on ice. The supernatant was removed and mixed with 275 μl 7 M ammonium acetate (pH 4) and incubated at 65° C. for 5 min. Chloroform (500 μl) was added and the mixture was vortexed for 15 sec prior to centrifugation (10 min, 4° C., 21 000×g). The supernatant was removed and the genomic DNA was precipitated with 1 volume of isopropanol for 5 min at room temperature. The DNA was recovered by centrifugation (10 min, 4° C., 21 000×g) and the resulting pellet was washed with 70% (v/v) ethanol. The sample was centrifuged (5 min, 4° C., 21 000×g) after which the ethanol was aspirated and the pellet dried under vacuum in a SpeedVac (Savant, USA). The pellet containing the isolated DNA was dissolved in 50 μl TE buffer [10 mM Tris (pH 7.8) and 1 mM EDTA] containing 5 mg/ml RNase and stored at −20° C. for future use.

Amplification of the XPR2^{p} Region From Y. lipolytica, the Endoglucanase I Signal Peptide Region from T. Reesei and the Xpr2^{p} Including the Pre-Pro Region from Y. lipolytica

Isolated genomic DNA from Y. lipolytica and T. reesei was used as template during a PCR to amplify the functional part of the XPR2^{p} from Y. lipolytica, the XPR2^{p} including the prepro-region as signal peptide (FIG. 5) and the partial endoglucanase I coding sequence (containing the 66 bp signal peptide) from T. reesei (FIG. 6). PCR amplification of the Y. lipolytica XPR2^{p}, the partial T. reesei endoglucanase I coding sequence and the XPR2^{p} containing the prepro-region entailed the use of primers XPR2-1F and XPR2-1R, TRsigP-1F and TRsigP-1R and XPR2-1F and XPR2(pre-pro)-1R (Table 7), respectively. The reaction mixture was subjected to thermal cycling as previously described with annealing of all primers at 57° C. for 30 sec.

Cloning of the XPR2^{p} and the Signal Peptides into the Shuttle Vector

To obtain heterologous expression of the EH coding sequences in Y. lipolytica, the plasmid JM62/64 was chosen as a basic shuttle vector to be used as a backbone to construct an expression vector containing the highly inducible XPR2^{p} promoter. The original POX2P promoter in the native plasmid was replaced with the XPR2^{p}, since the XPR2^{p} was shown to be among the strongest native promoters present in Y. lipolytica (Madzak et al., 2000). This was accomplished through the removal of the original POX2^{p} using restriction enzymes ClaI and BamHI and replaced with the PCR amplified XPR2^{p} region containing ClaI and BamHI flanking restriction sites. To verify the presence of the XPR2^{p} in the new vector (designated pMic62), the vector was digested with EcoRI and EcoRV and the presence of the new promoter was confirmed by restriction analysis of the PCR products.

Cloning of the Endoglucanase I Signal Peptide into the pMic62 Shuttle Vector

The pMic62 plasmid contained the highly inducible XPR2^{p} promoter to drive protein expression, but was still hampered since no secretion signal was present to direct the protein to the extracellular environment. The endoglucanase I signal peptide from T. reesei was cloned into the pMic62 vector to direct protein to the outside of the cell.

Cloning of the partial endoglucanase I coding sequence into the pMic62 vector was achieved by ligation of the digested partial endoglucanase I coding sequence (carrying BamHI and BlnI restriction sites at the 5′ and 3′ ends respectively) into the BamHI/BlnI digested pMic62 plasmid.

Removal of the rest of the unwanted regions (all but the 66 bp signal peptide) of the endoglucanase I coding sequence entailed using primers Constr-1F and Constr-1R (Table 7) in a PCR reaction.

The PCR was performed in a total volume of 50 μl containing 0.5 μl plasmid DNA (±250 ng), 2 pmol of each primer, 0.2 mM of each dNTP (dATP, dTTP, dCTP, dGTP) 5 μl of PCR buffer 2, 41 μl of nuclease free water and 5 units of Expand Long Template High Fidelity DNA polymerase (added after initial denaturation during thermal cycling). Thermal cycling consisted of denaturation for 5 min at 94° C. followed by 30 cycles of denaturation (94° C. for 15 sec), annealing of primers (58° C. for 30 sec) elongation (68° C. for 5 min with extended elongation time of 20 sec per cycle). A final step of 10 min at 68° C. was performed to complete elongation of the amplified product. The PCR product was ligated into plasmid vector pGem-T® Easy and designated Chimeric plasmid.

The resulting ˜6 kb fragment (containing DraI and BlnI restriction sites at the 5′ and 3′ ends respectively) was ligated into pGem-T® Easy forming a ˜9 kb chimeric plasmid. The ligation was performed to propagate the pMic62/64-TRsigP expression vector in E. coli cells, since DraI and BlnI do not have compatible sites to circularize the PCR fragment for self-propagation in E. coli. However, digestion of the chimeric plasmid using DraI and BlnI liberated the ˜5.4 kb pMic62/64-TRsigP shuttle vector (harboring restriction sites DraI at the 5′ end and BlizI at the 3′ end). This was verified by restriction digestion of the pMic62/64-TRsigP with DraI/BlnI.

The PCR amplified region containing the XPR2^{p} including the prepro-region (containing the ClaI and BamHI restriction sites at the 5′ and 3′ sites respectively) was ligated into pGem-T® Easy and propagated in E. coli. Insertion of the prepro-region of the XPR2 coding sequence into the pMic62-TRsigP+46 EH plasmid entailed the partial replacement of the XPR2^{p} with the NdeI and DraI digested pGem-T® Easy vector carrying the 1375 bp XPR2^{p} and the prepro-region.

The pMic62/64-TRsigP expression vectors contained the DraI restriction site directly in frame with the endoglucanase I signal peptide, with the BlnI restriction site at the region downstream of the DraI site for insertion of the coding sequence of interest under control of the promoter.

A blunt end (the DraI site) was purposely introduced to allow more flexibility in terms of compatible sites, since the construction of the vector limited the multiple cloning site (MCS) to only DraI and BlnI. The blunt end generated by the DraI digestion would allow the ligation of any blunt end to it, increasing the amount of restriction enzymes to be used for ligation of the 5′ end directly in frame with the signal peptide. The DraI/BlnI site of insertion makes the insertion of the coding sequence of interest possible without any orientation problems, since the overhangs generated upon digestion are not compatible and would not allow self-ligation of the 5′ and 3′ ends of the digested plasmid.

Cloning of the EH Coding Sequences from R. toruloides #46 into pMic62

The amplification of the EH from R toruloides (#46) was performed using primers EPH1-1F and EPH1-1R to introduce the DraI and BlnI sites respectively resulted in a product of ˜1200 bp. The product was ligated into pGem-T® Easy, transformed and propagated. The plasmid containing the correct insert, together with pMic62 were digested with DraI and BlnI and ligated into the expression vector carrying the XPR2^{p} to drive the expression of the proteins. The resulting vector containing the T. reesei endoglucanase I signal peptide (pMic62/64-TRsigP) was designated pMic62/64-TRsigP+46EH.

Insertion of the prepro-region of the XPR2 coding sequence into the pMic62-TRsigP+46 EH plasmid, to replace the T. reesei endoglucanase I signal peptide, entailed the partial replacement of the XPR2^{p} with the NdeI and DraI digested pGem-T® Easy vector carrying the 1375 bp XPR2^{p} and the prepro-region.

2. Construction of a Multi-Copy Plasmid (pYL-HmL=pINA 1293) Containing the Quasi-Constitutive hp4d^{p} and the Native Yarrowia lipolytica LIP2 Signal Peptide

pYL-HmL-pINA 1293 was obtained from Dr. Catherine Madzak of laboratory de Génétique, INRA, CNRS, France. This vector was renamed pYLHrnL (Yarrowia Lipolytica expression vector, with Hpd4 promoter, Multi-copy integration selection, L=LIP2 secretion signal)

Cloning of the Epoxide Hydrolase Coding Sequences from R. toruloides (#46), R. paludigenum(#692) and R. araucariae (#25) into pINA 1293

The EH coding sequences from R. toruloides and R. paludigenum were amplified using primers EPH1-1F (BamHI) and EPH1-1R (BlnI), 692cDNA-1F (BamHI) and 692cDNA-1R (NheI) (Table 7), respectively. The NheI restriction site was introduced into the sequence of the R. paludigenum EH by means of primer 692-cDNA-2R (Table 7), since a BlnI site could not be introduced at the 3′ end of the coding sequence due to the presence of a BlnI restriction site in halfway into the EH coding sequence. NheI restriction yielded a 3′ end compatible to the 5′ end of the plasmid after digestion with BlnI. Upon ligation of the compatible ends, the BlnI/NheI sites were destroyed with no other new useful site occurring.

The amplified products were ligated into pGem-T® Easy vector. The pGem-T® Easy vectors containing the EH enzymes from R. toruloides (containing the BamHI and BlnI restriction sites) and R. araucariae (containing the BamHI and BlnI restriction sites) were digested using a combination of BamHI and BlnI to release the EH insert from the plasmid backbone. The EH from R. paludigenum ligated into pGem-T® Easy (containing the BamHI and NheI restriction sites) was liberated from the plasmid backbone by digestion of the plasmid with a combination of BamHI and NheI.

The liberated EH encoding fragments were ligated into linearized pINA1293 plasmids (linearized using BamHI and BlnI) as previously described. Correct clones carrying the EH from R. toruloides were designated pINA1293+46 EH (=pYL-46HmL); R. paludigenum were designated pINA1293+692 EH (=pYL-692HmL), and R. araucariae were designated pINA1293+25 EH (=pYL-25HmL).

Restriction analysis performed on the various plasmids (carrying the different EH coding sequences) using different combinations of enzymes revealed the correctness of the constructs in terms or orientation and presence of signal peptides.

Verification of the Linkage Between the Signal Peptides and the Respective EH Encoding Coding Sequences in the pMic Plasmids and YL-HmL Plasmids

Sequence analysis of the all the constructs carrying the EH coding sequences revealed the correct ligation of the signal peptide in frame with the EH coding sequence located downstream of the relevant restriction site (Table 8).

TABLE 8

Verification of the linkage between the signal
peptides and the respective EH encoding coding sequences

| | Signal | Restric- | | |
|---|---|---|---|---|
| Plasmid peptide | tion site | EH origin | Deduced protein sequence | |
| pMic62/64- | Endo- | DraI | R. toruloides | ...ILAIARLVAAFKMATHTFAS |
| TRsigP + 46 EH | glucanase I | | | (SEQ ID NO:47) |
| pMic62/64- | XPR2 | DraI | R. toruloides | ...EIPASSNAKRFKMATHTFAS |
| prepro + 46 EH | | | | (SEQ ID NO:48) |
| pYL-25HmL | LIP2 | BamHI | R. araucariae | ...SEAAVLQKRFGSMSEHSFEA |
| | | | | (SEQ ID NO:49) |
| pYL-46HmL | LIP2 | BamHI | R. toruloides | ...SEAAVLQKRFGSMATHTFAS |
| | | | | (SEQ ID NO:50) |
| pYL-692HmL | LIP2 | BamHI | R. paludigenum | ...SEAAVLQKRFGSMAAHSFTA |
| | | | | (SEQ ID NO:51) |

The nucleotide sequences were translated into protein sequences using DNAssist Ver. 2.0. The deduced amino acid sequences of the signal peptides, restriction sites introduced and EH are italicized, underlined and illustrated in bold, respectively.

3. Construction of a Single-Copy Plasmid (pYL-TsA) Containing the Constitutive TEF^{p} and No Signal Peptide

The quasi-constitutive hp4d promoter (Madzak et al., 2000) was replaced with the constitutive TEF promoter (Müller et al., 1998) in the mono-integrative plasmid pINA1313 (Nicaud et al., 2002). The use of the TEF promoter aided in the activity screening experiments, since the hp4d promoter is growth phase dependent (only active from early stationary phase), whereas the TEF promoter drives constitutive expression to limit induction differences between yeasts grown during activity screening and on flask scale.

The hp4d promoter in pINA1313 was replaced with the TEF promoter using ClaI and HindIII restriction sites, followed by the PCR removal of the LIP2 signal peptide using primers-sigp-1F and -sigP-1R. The purified PCR mixture was treated with BamHI and HindIII (where HindIII digested the template DNA but not the PCR product) to prevent recircularization of the template DNA, thereby preventing concomitant template contamination of transformation mix upon ligation. The PCR product was allowed to circularize using T4 DNA ligase to join the compatible BainHI ends resulting in plasmid pKOV96=pYLTsA.

The EH coding sequences of #23, #25, #46 and #692 were amplified as described in Example 1.

The amplified EH coding sequences and the pKOV96=pYLTsA plasmid were digested with the appropriate restriction enzymes to create compatible cohesive ends suitable for ligation of the EH coding sequences into the BamHI-AvrII cloning sites of the plasmid, resulting in plasmids pYL-23TsA, pYL-25TsA, pYL-46TsA and pYL-692TsA.

Transformation of Integrative Vectors into Y. lipolytica

NotI linearized pMic62-TrsigP, pMic62pre-pro, pKOV96 (=pYL-TsA) and pYL-HmL integrative vectors (containing the different EH encoding coding sequences), were used to transform Y. lipolytica strains Po1d and Po1h, respectively. Transformation was performed as essentially described by Xuan et al. (1988).

The Po1h and Po1d transformants were grown on selective YNB casamino acid media [YNB without amino acids and ammonium sulfate (1.7 g/l), NH₄Cl (4 g/l), glucose (20 g/l), casamino acids (2 g/l). and agar (15 g/l)]. Colonies were isolated after 2-15 days of incubation at 28° C. as positive transformants containing the integrated expression cassette.

For the transformants carrying the hp4d^{p} (pYL-HmL) and TEF^{p} (pYL-TsA), cells were cultivated in flasks containing ⅛^{th} volume YPD medium at 28° C. with shaking. The cells were harvested by centrifugation (5 min, 4° C., 5000×g) and the cellular fraction was separated from the supernatant. The cellular fraction was washed and suspended in phosphate buffer (50 mM, pH 7.5, containing 20% (v/v) glycerol) to a final concentration of 20% (w/v). Glycerol was added to the supernatant to a final concentration of 20% (v/v) and the pH was adjusted to 7.5 using 1M HCl. The cellular and supernatant fractions were frozen at −20° C. for future use.

Y. lipolytica Po1d or Po1h transformants carrying the integrants containing the XPR2^{p} (pMic62TrsigP and pMic62pre-pro) were cultivated in ⅛^{th} volume liquid YPD medium in 500 ml shake flasks for 30 hours (late exponential to early stationary phase) at 28° C. The cells were harvested by centrifugation (5000×g for 5 min, twice washed with phosphate buffered saline (PBS) (Sambrook et al., 1989) and suspended in GPP medium that was used for recombinant EH production medium. The cells were incubated while shaking at 28° C. for 24 hours. After induction, the cells were harvested by centrifugation and the cellular fraction was separated from the supernatant. The cells were suspended to a concentration of 20% (w/v) using 50 mM phosphate buffer (pH 7.5) containing 20% (v/v) glycerol and the pH of the supernatant was adjusted to 7.5 using 1 M NaOH.

As an alternative to the GPP medium used for induction of the XPR2^{p}, modified full inducing YPDm medium (0.2% yeast extract, 0.1% glucose and 5% proteose peptone) (Nicaud et al., 1991) was also used to induce the XPR2^{p} where cells were cultivated in the YPDm media for 48 hours at 28° C. while shaking.

### Example 3

### Cloning and Overexpression of an Epoxide Hydrolase that is Highly Active and Selective in the Native Host and in Yarrowia lipolytica into Saccharomyces cerevisiae

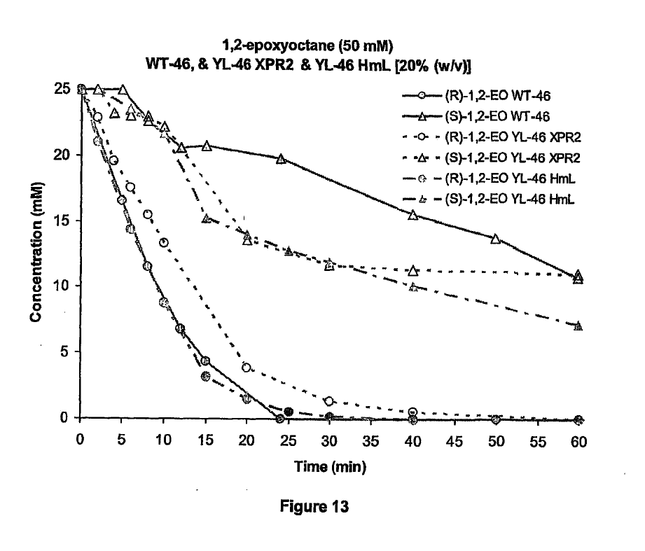

TABLE 9
Vectors, Strains, and Oligonucleotide Primers

| | | Reference/ |
|---|---|---|
| Vectors | Description | Origin |
| | See FIG. 13. Shuttle vector for E. coli/S. cerevisiae. | |
| pYES2 | Prepared from Top 10F′E. coli containing the extra-chromosomal | In Vitrogen |
| | DNA | |
| pYL25HmL | Plasmid pINA1293 (= pYLHmL) containing the epoxide hydrolase | Above |
| | cDNA from Rhodotorida araucariae NCYC 3183 | |

Strains

| E. coli XL10 Gold® | | Strategene |
|---|---|---|
| E. coli Top10F′ | | In Vitrogen |
| Saccharomyces cerevisiae | | In Vitrogen |
| INVSc1 | | |
| | | Restriction |
| Primers | Sequence | site |

Primers designed for amplifying the cDNA insert from pYL25HmL

| | | | |
|---|---|---|---|
| EH8_EcoRI | 5′-GAG AAT TCT GAG GAG GAG AG-3′ | (SEQ ID NO:52) | EcoRI |
| EH5_BamHI | 5′-GTG GAT GGA TGA GGG AGG A-3′ | (SEQ ID NO:23) | Bam-HI |

Underlining indicates the sequence of introduced restriction sites.

Excising the Rhodotorula araucariae Epoxide Hydrolase (RAE1H) cDNA

The RAEH (R. araucariae NCYC 3183 epoxide hydrolase) coding sequence was initially cloned into a dual expression vector pYL25HmL (=pINA1293) containing a secretion peptide signal for secretion of the protein when expressed in Yarrowia expression system.

The primers EH8_EcoRI and EH5_BamHI (Table 9) were used for PCR amplification of the cDNA of the RAEH from pYL25HmL. A 1.3 kb amplicon was excised from an agorose gel and purified using the GFX PCR DNA and gel band Purification kit (Amersham). This purified RAEH DNA was digested overnight with EcoRI and BamHI to create complementary overhangs for ligation into pYES2 plasmid.

Ligation of RAEH cDNA into pYES2 Plasmid

The pYES2 parental vector DNA was prepared from a 10 ml LB overnight inoculum of Top10F′ E. coli containing the extra-chromosomal DNA plasmid. The purified plasmid was digested overnight with EcoRI and BamHI. RAEH cDNA and pYES2 were ligated at a pmol end ratio of 5:1 (Insert:vector) using T4 DNA ligase overnight at 16° C. The resultant pYES_RAEH plasmid ligation mixture was electroporated in electro-competent E. coli XL10 Gold cells using Bio-Rad's GenePulser according to the standard given protocol and plated onto LB ampicillin selection plates supplemented with ampicillin (100 μg/ml). Plasmid purification and restriction analysis was performed on transformants to determine the integrity of the construct. There resulting plasmid was designated pYES_RAEH.

Transformation of Saccharomyces cerevisiae INVSc1

pYES_RAEH plasmid DNA was isolated from E. coli XL10 Gold transformants and the constructs confirmed by restriction with XbaI and HindIII to excise the cloned cassette from the pYES2 vector (FIG. 2). S. cerevisiae INVScI was transformed with plasmid DNA by the lithium acetate/DMSO method. The transformed cells were plated onto selective media lacking uracil (SC Minimal Media containing 0.67% w/v yeast nitrogen base without amino acids and ammonium sulphate (Difco 233520), 0.5% w/v ammonium sulphate, 0.01% m/v of each of adenine, arginine, cysteine, leucine, lysine, threonine, tryptophan and 0.005% m/v of each of aspartic acid, histidine, isoleucine, methionine, phenylalanine, praline, serine, tyrosine and valine) and incubated for 48 hours at 30° C. 2% galactose was added to induce transcription of the RAEH under control of the GAL1 promoter), no uracil was included (for maintenance of the pYES2 plasmid) and the pH was not adjusted to neutral (and was approximately pH 5.0). Transformants of Saccharomyces cerevisiae were grown in SC Minimal Media. The Saccharomyces recombinants were grown in 50 ml media in 250 ml Erlenmeyer flasks shaking for 48 hours at 30° C. Cells were harvested by centrifugation, suspended in phosphate buffer (pH 7.5, 50 mM) to a concentration of 50% (wet mass/v) for immediate evaluation of enzyme activity without further storage.

### Example 4

### General Methods for Biocatalyst Production and Epoxide Hydrolase Mediated Biotransformations

Yarrowia transformants were grown in 50 ml YPD liquid media (1% m/v yeast extract, 2% m/v peptone, 2% m/v dextrose, pH 5.5-6.0) in a 250 ml Erlenmeyer flask for 3 days at 28° C. shaking at 200 rpm. The cells were harvested by centrifugation at 5000 rpm for 10 minutes under chilling and the pellet volume resuspended to 20% m/v in chilled 50 mM potassium phosphate buffer pH 7.5 for immediate evaluation of enzyme activity without further storage or with the addition of 20% m/v glycerol to the buffer for storage at −20° C. for later use.

Recombinant Saccharomyces cerevisiae constructs were grown in SC Minimal Media containing 0.67% m/v yeast nitrogen base without amino acids and ammonium sulphate (Difco 233520), 0.5% m/v ammonium sulphate, 2% galactose (to induce transcription of the RAEH under control of the GAL1 promoter), 0.01% m/v of each of adenine, arginine, cysteine, leucine, lysine, threonine, tryptophan and 0.005% m/v of each of aspartic acid, histidine, isoleucine, methionine, phenylalanine, praline, serine, tyrosine and valine. No uracil was included (for maintenance of the pYES2 plasmid) and the pH was not adjusted to neutral (and was approximately pH 5.0). The Saccharomyces recombinants were grown in 50 ml media in 250 ml Erlenmeyer flasks shaking for 48 hours at 30° C. Cells were harvested by centrifugation and suspended in phosphate buffer (pH 7.5, 50 mM) to a concentration of 50% (wet mass/v) for immediate evaluation of enzyme activity without further storage or with the addition of 20% m/v glycerol to the buffer for storage at −20° C. for later use.

Screening for transformants exhibiting epoxide hydrolase activity entailed the addition of racemic epoxide (2 μl) to 1 ml of the 20% ((m/v) in 50 mM phosphate buffer; pH 7.5)) cell suspension. For evaluation of epoxide hydrolase activity in the culture supernatants, the supernatants from centrifugation were diluted 9:1 with 50 mM phosphate buffer pH 7.5 and used directly in the biotransformation by addition of the substrate without further dilution. Non-chiral TLC was performed as described below in this example.

For evaluation of epoxide hydrolase characteristics of whole cell biocatalysts, Y. lipolytica transformants and Saccharomyces transformants were grown as described above in this example. Biotransformations were conducted in 50 mM pH 7.5 potassium phosphate buffer together with the racemic epoxide under study and incubated under vortex mixing in sealed glass vials at temperatures and biomass loadings described in the specific example figures. The biomass loadings described in the figures refer to the % v/v of wet weight biomass cell suspension present in the biotransformation matrix excluding the volume of the epoxide substrate. The racemic epoxide was usually added directly (1,2-epoxyoctane, styrene oxide) or as a stock solution in EtOH (i.e., indene oxide, 2-methyl-3-phenyl-1,2-epoxypropane, cyclohexene oxide).

After suitable incubations, samples were removed and extracted with ethyl acetate or the reactions were stopped by the addition of ethyl acetate to 60% of the reaction volume, vortexed for 1 minute, and centrifuged at 13 000 rpm for 5 min. The solvent layer was dried over anhydrous magnesium sulphate and analysed by TLC for presence of activity and HPLC (high pressure liquid chromatography) or GC (gas chromatography) for chiral analysis.

Non-chiral TLC was performed using commercially available silica gel plates (Merk 5554 DC Alufolien 60 F₂₅₄) as the stationary phase and chloroform:ethylacetate [1:1 (v/v)] as the mobile phase. Ceric sulphate (ceric sulphate saturated with 15% H₂SO₄) or vanillin stain [2% (w/v) vanillin, 4% (v/v) H₂SO₄ dissolved in absolute ethanol] was used as a spray reagent to visualize the residual epoxide and formed diol.

Chiral GC was performed on a Hewlett Packard 5890-series II gas chromatograph equipped with a FID detector and an Aligent 6890-series autosampler-injector, using hydrogen as a carrier gas at a constant column head pressure of 140 kPa. Quantitative analysis of the enantiomers of 1,2-epoxyoctane and 1,2-octanediol was achieved using a Chiraldex A-TA chiral fused silica cyclodextrin capillary column (supplied by Supelco) at oven temperatures of 40° C. and 115° C., respectively. Quantitative chiral analysis of cyclohexane diol was achieved using GC using a β-DEX 225™ fused silica cyclodextrin capillary column (Supelco) (30 m length, 25 mm id, 25 um film thickness).

Quantitative chiral analysis of styrene oxide and 3-chlorostyrene oxide was achieved using GC using a β-DEX 225™ fused silica cyclodextrin capillary column (Supelco) (30 m length, 25 mm id, 25 um film thickness) oven temperatures of 90° C. and 100° C., respectively. Quantitative chiral analysis of 2-methyl-3-phenyl-1,2-epoxpropane and 2-methyl-3-phenyl-propanediol was performed by GLC using a fused silica β-DEX 110 cyclodextrin capillary column (Supelco) (30 m length, 25 mm ID and 25 μm film thickness). The initial temperature of 80° C. was maintained for 22 minutes, increased at a rate of 4° C. per minute to 160° C., and maintained at this temperature for 1 minute. The retention times (min) were as follows: Rₜ (S)-epoxide=31.9, Rₜ (R)-epoxide=32.1, Rₜ (S)-diol=47.7., Rₜ (R)-diol=48.0.

Chiral HPLC was performed on a Hewlett Packard HP1100 equipped with UV detection. Quantitative chiral HPLC analysis of indene oxide enantiomers was achieved using a Chiracel OB-H, 5u, 20 cm×4.6 mm, S/N OBHOCE-DK024 column at 25° C. using 90% n-Hexane (95% HPLC grade)+10% ethanol (99.9% AR) eluent.

### Example 5

### Functional Expression of Epoxide Hydrolases from all Sources in Yarrowia lipolytica (YL-sTsA Transformants) and Direct Comparison of the Activity and Selectivity of the Different Enzymes for the Resolution of Epoxides

### Qualitative Epoxide Hydrolase Activity Analysis

Chiral quantitative analysis for EH activity was performed on transformants cultivated in liquid YPD for 48 hours. Harvested cells were washed with and suspended in 50 mM phosphate buffer (pH 7.5) to a final concentration of 10% or 20% (w/v). Reactions were started by addition of the substrate to a final concentration of 10 or 100 mM and the mixtures were incubated in a carousel stirrer at 25° C. Samples (300 μl) were taken at regular intervals, extracted with 500 μl ethylacetate, centrifuged (10 min, 10 000×g), after which the organic layers were removed (ethylacetate fraction was dried using MgSO₄) and analyzed as described in Example 4.

Comparison of the Activity and Selectivity of YL-sTSA Transformants for 2-methyl-3-phenyl-1,2-epoxypropane

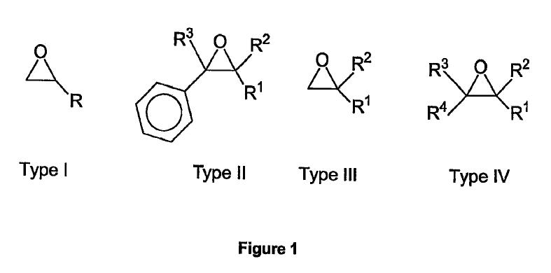

Biotransformations were performed with 20% (w/v) wet weight cells and 10 mM racemic 2-methyl-3-phenyl-1,2-epoxypropane (a 2,2-disubstituted epoxide-Type III, see FIG. 1). The course of the reactions were followed by extracting samples at suitable time intervals over 180 minutes as described above and analysed by chiRal GC.

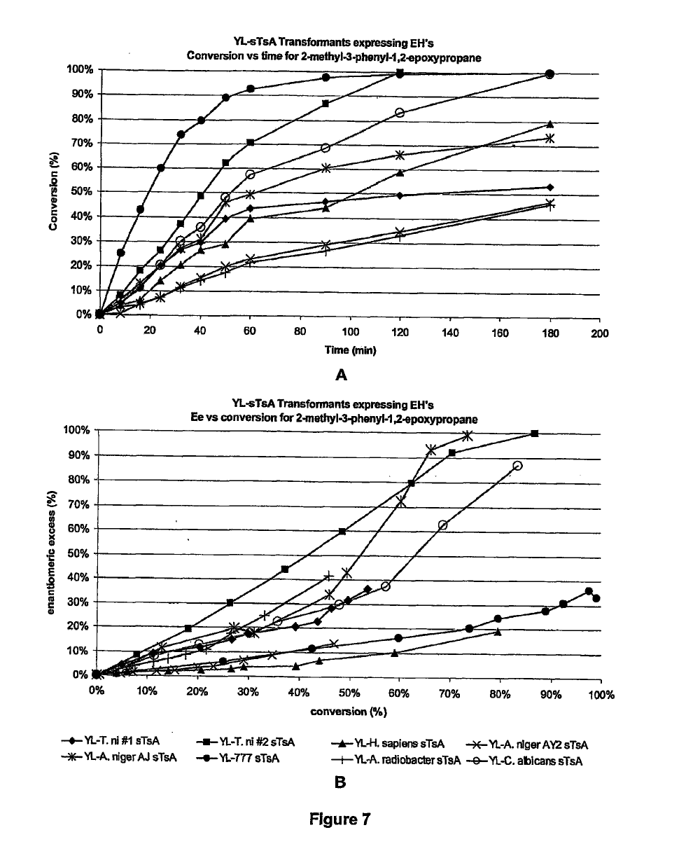

All YL-sTsA transformants displayed functional EH activity. The activities of the transformants harboring the EH coding sequences from the different sources were evaluated by plotting a graph of the conversion against time (FIG. 7A). The selectivities of the transformants harboring the EH coding sequences from the different sources were evaluated by plotting a graph of the enantiomeric excesses at different conversions (FIG. 7B). From these graphs the catalyst with the desired activity and selectivity can be selected. For example, from FIG. 7A it can be seen that YL-T. ni # 2 sTsA reached 50% conversion after 40 minutes, which is approximately double the time for YL-777 sTsA to reach 50% conversion. However, from FIG. 7B it is clear that the enantiomeric excess at 50% conversion of the epoxide catalysed by YL-T. ni # 2 sTsA is substantially higher than that of YL-777 sTsA. Since the EH coding sequences are expressed as single copies in the same location of the genome of the host cells and under control of the same promoter, this expression sytem can be used to select the most suitable enzyme for any given epoxide based on the kinetic properties required.

Selection of the Most Suitable Catalyst for the Enantioselective Hydrolysis of 1,2-epoxyoctane

Biotransformations were performed with 10% (w/v) wet weight cells and 100 mM racemic 1,2-epoxyoctane. Only YL-sTsA transformants harboring the more highly active microsomal EH from yeasts #23, #25, #46, #692 and #777 displayed substantial hydrolysis of the epoxide at this concentration. Biotransfromations for the YL-sTsA transformants haroring EH coding sequences from other sources were repeated with 10 mM 1,2-epoxyoctane to determine initial rates over the same time period as that of the YL-sTsA transformants harboring microsomal yeast EH. The course of the reactions were followed by extracting samples at suitable time intervals as described above and analysed by chiral GC.

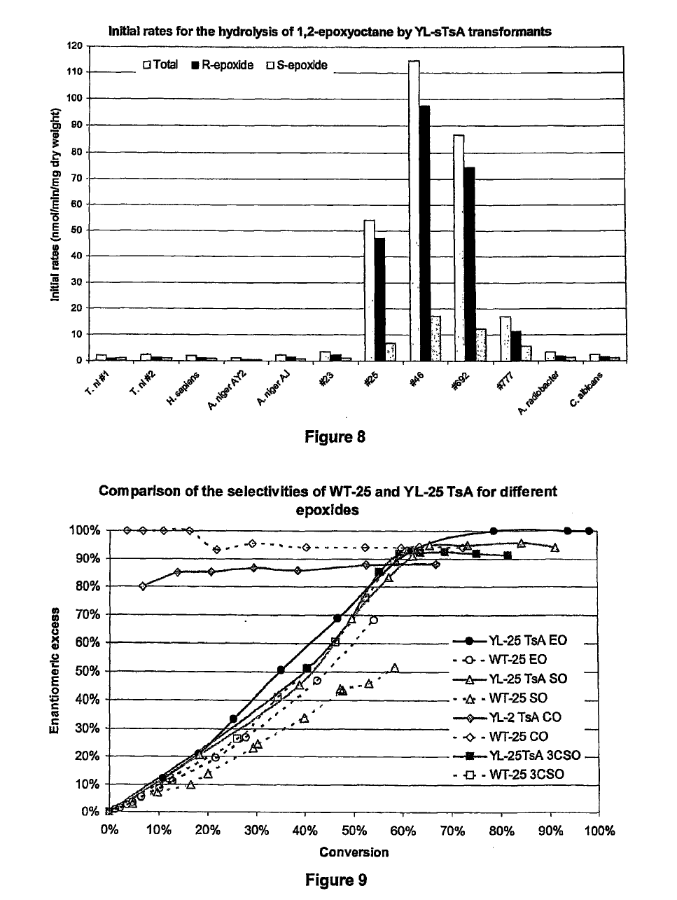

Initial rates of hydrolysis of the different YL-sTsA transformants for the racemic epoxide and the R- and S-enantiomers were plotted (FIG. 8). From this graph, the catalysts with the highest activities (highest total rate of hydrolsysis) as well as the highest selectivities (highest difference between initial rates of R- and S-enantiomers) can be selected unbiased, since the conditions of expression are uniform. For example, the YL-sTsA transformants harboring the microsomal yeast EH of #25, #46 and #692 displayed much higher rates and selectivities for 1,2-epoxyoctane than the YL-sTsA transformants expressing EH from other sources.

### Example 6

### Comparison of the Expression of Epoxide Hydrolases in the Different Yeast Host Strains Yarrowia lipolytica and Saccharomyces cerevisiae

The EH from Rhodotorula araucariae (#25, NCYC 3183) was selected to determine if functional expression with comparable activities and selectivities to that of the wild type enzyme could be obtained in different yeast expression systems. This EH displayed excellent activity and selectivity for a wide range of substrates in the wild type. The enzyme was expressed under control of a constitutive promoter (TEF^{p}) as a single copy construct in Yarrowia lipolytica (pYL-TsA integrative plasmid) as well as in Sacharomyces cerevisiae under control of the GAL1^{p} (pYES2 plasmid) as described above. Functional expression under the suitable growth conditions for induction of expression in S. cerevisiae and normal growth conditions in YPD media for the Y. lipolytica transformant and the wild type yeast was evaluated and compared for the two expression hosts as well as that of the wild type enzyme for different epoxides.

The wild type enzyme (WT-25) and the recombinant enzyme (YL-25 TsA) were compared in biotransformations with 1,2-epoxyoctane (EO) a monosubstituted epoxide (Type I in FIG. 1), styrene oxide (SO) and 3-chlorostyrene oxide (3CSO) (styrene type epoxides-Type II in FIG. 1) cyclohexene oxide (CO) (a cis-2,3-disubstituted epoxide as in Type IV in FIG. 1, where R₂═R₃═H and R1 and R4 together is a cyclohexene ring). The conditions for the biotransformation reactions are given in Table 10. While differences in activities were observed between the WT enzyme and the recombinant enzyme as expected, good comparison between the selectivity of the wild type EH and the enzyme expressed in Y. lipolytica was obtained for all epoxides (1,2-epoxyoctane, styrene oxide and cyclohexene oxide) (FIG. 9).

TABLE 10
Reaction conditions used for WT-25 and YL-25 TsA biotransformations

| | WT-25 | | YL-25 TsA | |
|---|---|---|---|---|
| | [biomass] | [substrate] | [biomass] | [substrate] |
| Epoxide | % (w/v) | (mM) | % (w/v) | (mM) |
| 1,2-epoxyoctane | 10 | 100 | 10 | 100 |
| Styrene oxide | 50 | 50 | 20 | 100 |
| Cyclohexene oxide | 50 | 50 | 50 | 50 |
| 3-chlorostyrene oxide | 50 | 50 | 50 | 50 |

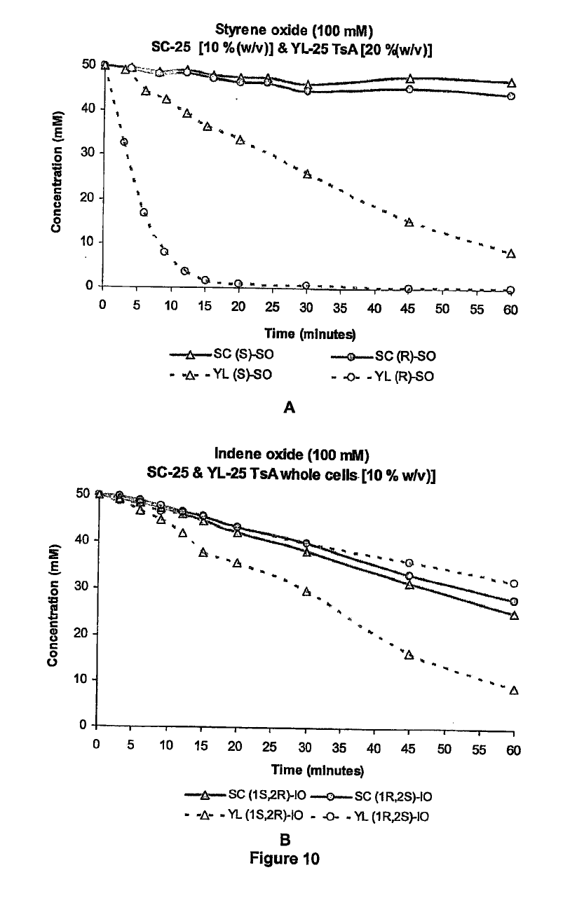

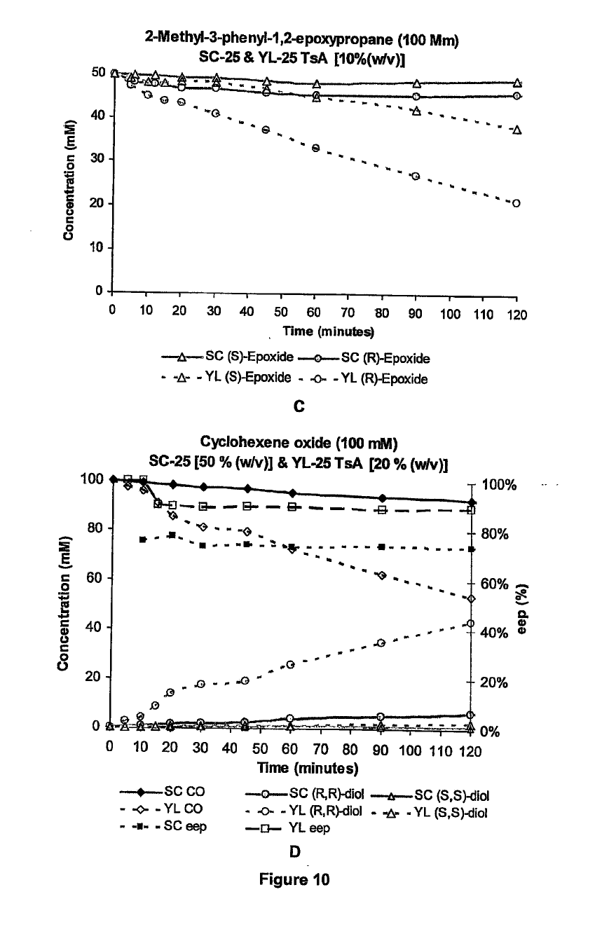

The recombinant enzyme expressed in S. cerevisiae (SC-25) and Y. lipolytica (YL-25 TsA) were compared in biotransformations with styrene oxide (SO) (FIG. 10A), indene oxide (IO) (FIG. 10B), 2-methyl-3-phenyl-1,2-epoxypropane (MPEP) (FIG. 10C) and cyclohexene oxide (CO) (FIG. 10D). The conditions for the biotransformation reactions are given in the figures.

While the kinetic properties of the WT enzyme remained substantially unchanged or were slightly enhanced when expressed in Y. lipolytica, activity as well as selectivity of the recombinant enzyme expressed in S. cerevisiae decreased compared to the recombinant enzyme expressed in Y. lipolytica for all epoxides tested (FIGS. 10A, 10B, 10C, and 10D).

It is known that Saccharomyces cerevisiae hyper-glycosylates foreign proteins which may sterically hinder the epoxide hydrolase. The results shown here illustrate that intracellular production of yeast derived epoxide hydrolase in the recombinant host Yarrowia lipolytica is highly suitable for production of stereoselective biocatalysts for application to resolution of racemic epoxides as compared to the other expression hosts.

### Example 7

### Comparison of Kinetic Properties of Epoxide Hydrolases of Yeast Origin as Expressed in Recombinant Yarrowia lipolytica with and without Direction by Different Secretion Signal Peptides for 1,2-epoxyoctane and the Effects on Localization of the Recombinant EH

Positive transformants were inoculated into 5 ml YPD and grown while shaking at 28° C. for 48 hours. Cells (1 ml) were centrifuged (5 min at 13 000×g), followed by aspiration of the supernatant. The pellet was resuspended in 750 μl of a 50 mM phosphate buffer (pH 7.5). Epoxide (2 μl) was added to 1 ml of the cell suspension, followed by incubation while shaking at 25° C. for 60 min. The remaining epoxide and newly formed diol were extracted from the reaction mixtures with 300 μl ethylacetate. After centrifugation (5 min, 10 000×g), diol formation was evaluated by thin layer chromatography (TLC).

(a) Evaluation of the Activity of the Recombinant EH from Rhodospordium toruloides (#46, UOFS Y-0471) Expressed in Y. lipolytica Under Control of the Inducible XPR2 Promoter and Containing the Signal Peptides from Trichoderma reesei Endoglucanase 1 and the XPR2 Pre-Pro Region, Respectively.

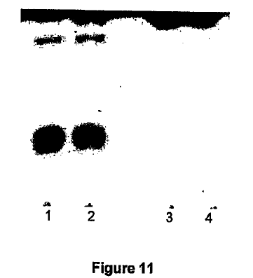

Whole cells and supernatants of YL-46 Mic62TRsigP (Y. lipolitica strain Po1h transformed with the pMic62 single copy integrative plasmid under control of the XPR2 promoter and containing the coding sequence from #46 and the T. reesei signal peptide) and YL-46Mic62pre-pro transformants (Y. lipolitica strain Po1h transformed with the pMic62 single copy integrative plasmid under control of the XPR2 promoter and containing the coding sequence from #46 and the XPR2 pre-pro signal peptide) were evaluated for EH activity against 1,2-epoxyoctane, an epoxide for which the WT #46 displays good activity and selectivity (FIG. 11). Good activity was observed in both the cellular fractions and supernantants with the T. reesei signal peptide while very low cellular activity was observed with the LIP2 pre-pro region signal peptide. Thus, quantitative analysis was only performed for the transformant with the T. reesei signal peptide.

(b) Evaluation of the Activity of the Recombinant EH from R. araucariae (#25), R. toruloides (#6), and R. paludigetum (#692) Expressed in Y. lipolytica Under Control of the hp4d Promoter and Containing the LIP2 Signal Peptide (YL-HML Transformants).

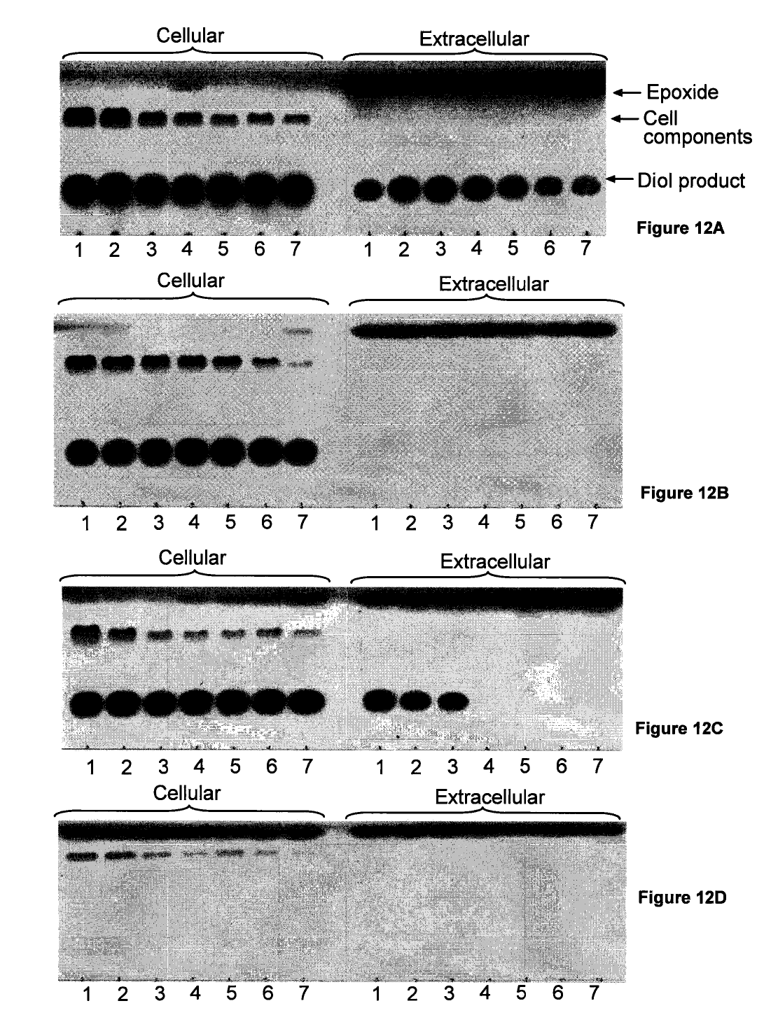

Whole cells and supernatants of YL-25 HmL, YL-46 HmL and YL-692 HmL (Y. lipolitica strain Po1h transformed with the multi-copy integrative plasmid pINA 1293=pYL-HmL under control of the hp4d promoter and containing the coding sequences from #25, #46 and #692, respectively, as well as the LIP2 secretion signal from Y. lipolytica) were evaluated for EH activity with the 1,2-epoxyoctane substrate. Biotransformations were performed on the transformants cultivated for 8 days (7 days after stationary growth phase was reached) in YPD at 28° C. One day (24 hours) after stationary phase was reached, cells carrying the multi copy integrants under control of the hp4d^{p} were able to achieve the intracellular expression of the coding sequence products from day 1 to day seven (FIG. 12). Extracellular expression of the recombinant EH enzymes was only obtained for the EH from R. araucariae and R. paludigenum (FIGS. 12A and 12C, respectively). Therefore, active EH could be expressed with a variety of signal peptides, but the cellular localization remained mainly intracellular.

(c) Evaluation of the Effect of Signal Peptides on the Activity and Selectivity of the Recombinant EH From R. araucariae (#25), R. toruloides (#6), R. paludigenum (#692) Expressed in Y. lipolytica During the Hydrolysis of 1,2-epoxyoctane

Chiral quantitative analysis for EH activity was performed on transformants cultivated in liquid YPD for 48 hours. Harvested cells were washed with and suspended in 50 mM phosphate buffer (pH 7.5) to a final concentration of 10% or 20% (w/v). Reactions were started by addition of the substrate to a final concentration of 10 or 100 mM and the mixtures were incubated in a carousel stirrer at 25° C. Samples (300 μl) were taken at regular intervals, extracted with 500 μl ethylacetate, centrifuged (10 min, 10,000×g), after which the organic layers were removed (ethylacetate fraction was dried using MgSO₄), and analyzed as described in Example 4.

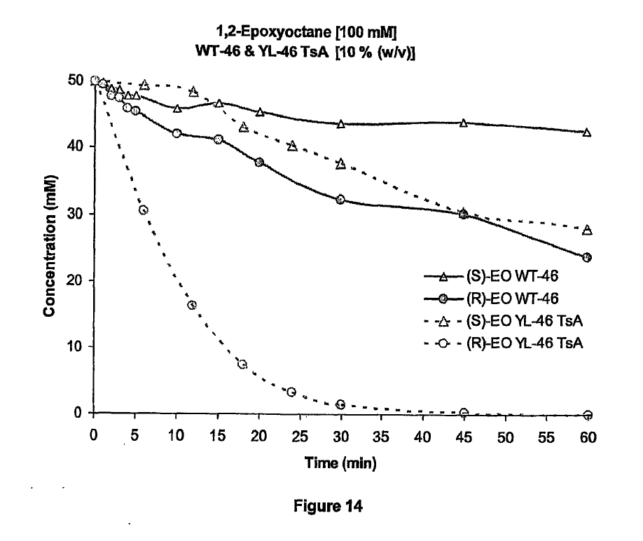

The kinetic properties (activity and selectivity) of the recombinant EH of #46 in the wild type (WT-46), and with signal peptides (YL-46 Mic62TRsigP (=YL-46×PR2) and YL-46 HmL) (FIG. 13) as well as without signal peptides (YL-46 TsA) (FIG. 14) were evaluated for the hydrolysis of 1,2-epoxyoctane. The presence of both signal peptides caused a decrease in the selectivity of the enzyme (FIG. 13). However, in the absence of a signal peptide, expression of the recombinant enzyme in Y. lipolytica, even in single copy, caused a dramatic increase in activity and selectivity compared to the wild type (FIG. 14).

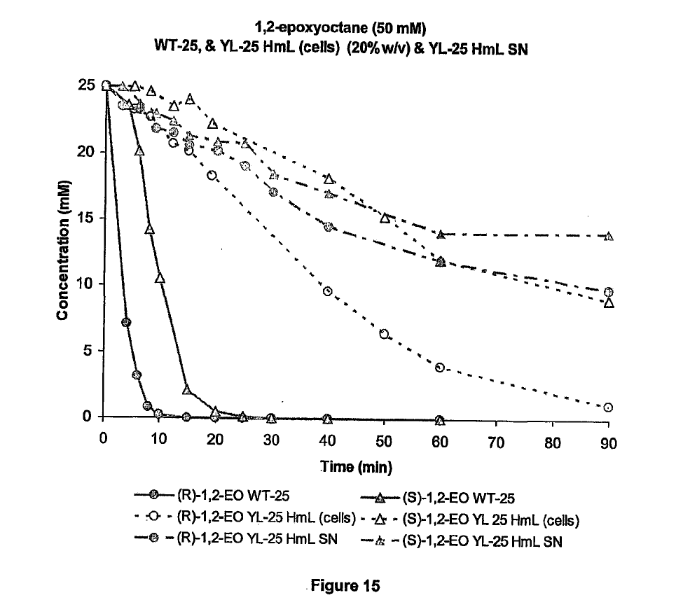

The recombinant Y. lipolytica strain expressing the EH from R. toruloides (#46), (YL-46 HmL) did not secrete any detectable EH into the supernatant. The kinetic properties of the secreted EH was determined using YL-25 HmL that secreted the most EH into the supernatant (see FIG. 12). The hydrolysis of 1,2-epoxyoctane was compared for the wild type strain (WT-25), the recombinant EH with the signal peptide retained intracellularly (YL-25 HmL cells) and the recombinant EH secreted into the supernatant (YL-25 HmL SN) (FIG. 15).

The whole cell biotransformations were carried out with 20% (w/v) cellular suspensions in 10 ml reaction volume, while the biotransformation with the SN was carried out using the entire SN fraction from a 25 ml shake flask from which the cells were harvested and concentrated by ultrafiltration to 10 ml reaction volume.

The recombinant EH with the signal peptide present retained intracellularly displayed a decrease in selectivity and activity compared to the WT-25 strain. Furthermore, the secreted enzyme in the supernatant fraction displayed almost a total loss of selectivity (FIG. 15).

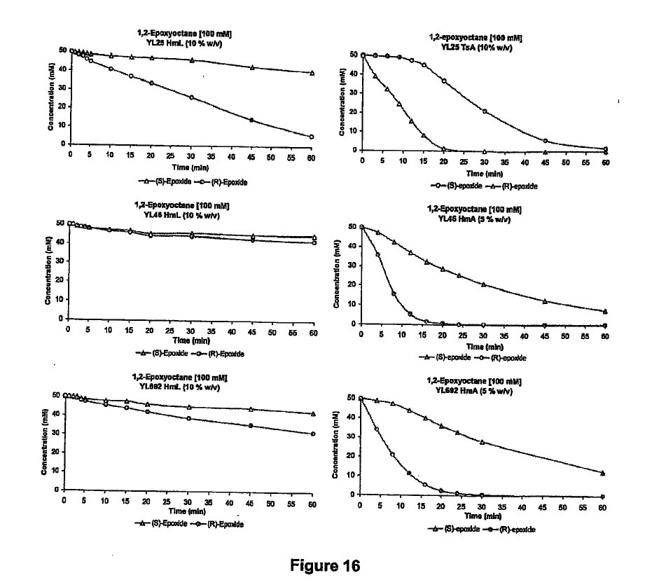

The effect on the activity and selectivity of multi-copy transformants with the LIP2 signal peptide present (YL-HmL transformants) and without the LIP2 signal peptide (YL-HmA transformants) was compared for other EH for 1,2-epoxyoctane to determine if the presence of a signal peptide lead to a decrease in activity and selectivity for the different EH. In all cases, the presence of the signal peptide caused a decrease in both the activity and selectivity of the recombinant EH (FIG. 16), even compared to single-copy transformants without the signal peptide (YL-25 TsA).

### Example 8

### Comparison of Kinetic Properties of Epoxide Hydrolases of Yeast Origin as Expressed in Recombinant Yarrowia lipolytica with and without a Signal Peptide for Different Epoxides

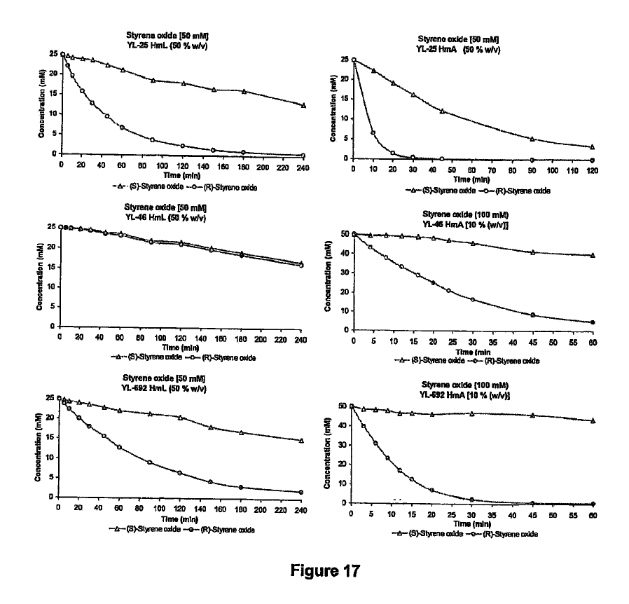

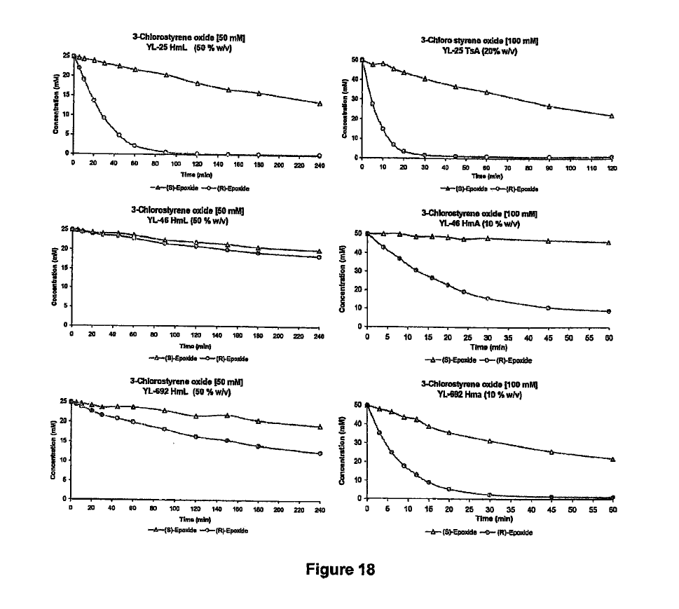

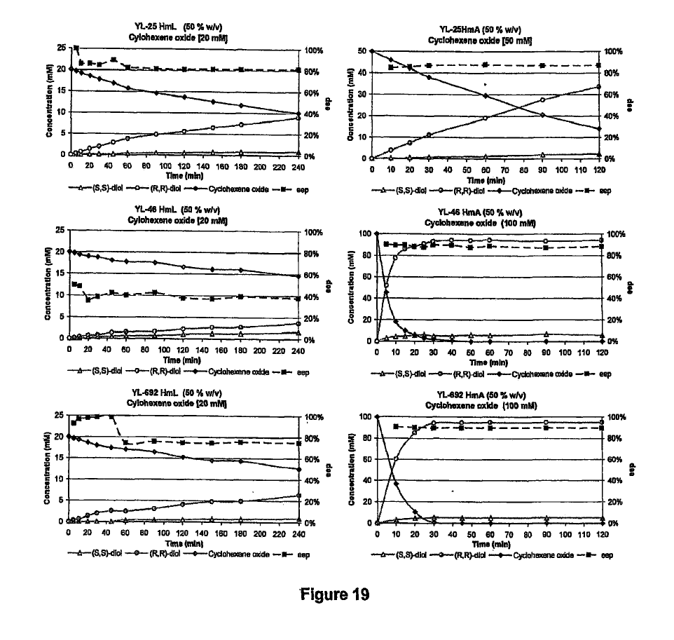

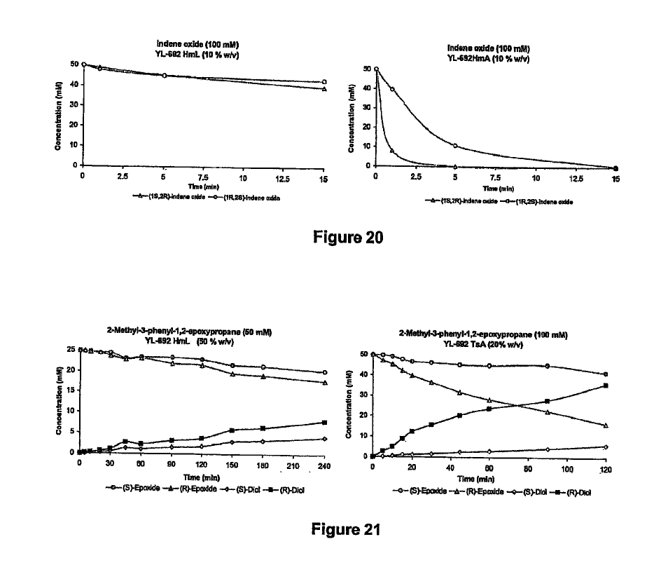

Biotransformations were performed to compare the activity and selectivity of different EH expressed in Y. lipolytica with and without signal peptides across a wide range of different epoxides to ascertain that the decrease in activity and selectivity observed for 1,2-epoxyoctane by recombinant EH containing a signal peptide, was a general phenomenon. The recombinant Y. lipolytica strains expressing EH containing a signal peptide (YL-25 HmL, YL-46 HmL, YL-692 HmL) and the recombinant Y. lipolytica strains expressing EH without a signal peptide (YL-25 HmA, YL-46 HmA, YL-692 HmA) were compared for the hydrolysis of styrene oxide (FIG. 17), 3-chlorostyrene oxide (FIG. 18) and cyclohexene oxide (FIG. 19). The recombinant strains YL-692 HmL and YL-692 HmA were also compared for indene oxide (FIG. 20) and 2-methyl-3-phenyl-1,2-epoxypropane (FIG. 21). The reaction conditions used during the biotransformations were as described in Example 4, and the substrate concentrations and biomass loadings used are given with each graph on the figures. Chiral analysis of the different epoxide enantiomers were performed as described in Example 4.

In all cases, for all strains and all epoxide substrates tested, the presence of a signal peptide caused a decrease in both the activity and selectivity of the recombinant EH.

Surprisingly, the advantageous kinetic characteristics of EH such as activity and selectivity were adversely affected and that the enzymes are predominantly retained within the cell, even with various secretion signal sequences attached, and that any EH enzyme that was secreted into the supernatant had lower selectivity and activity.

### Example 9

### Comparison of the Effect of Different Promoters (TEF^{p} and hp4d^{p}) on the Expression Level and Kinetic Properties of EH from Different Sources

Comparison of the kinetic properties of recombinant EH expressed in Yarrowia lipolytica Po1h host under control of the hp4d promoter and transformed with an integrative vector with the ura3d4 selective marker containing the various EH coding sequences (YL-HmA transformants) and the same recombinant EH expressed in Yarrowia lipolytica Po1h host under control of the TEF promoter transformed with an integrative vector with the ura3d1 selective marker containing the various EH coding sequences (YL-TsA transformants) was performed with a range of different epoxides to determine the efficiency of the different promoters and the effect of copy number on activity and selectivity of the enzymes.

Biotransformations were performed to compare the hydrolysis of different epoxides by YL-TsA and YL-HmA transformants.

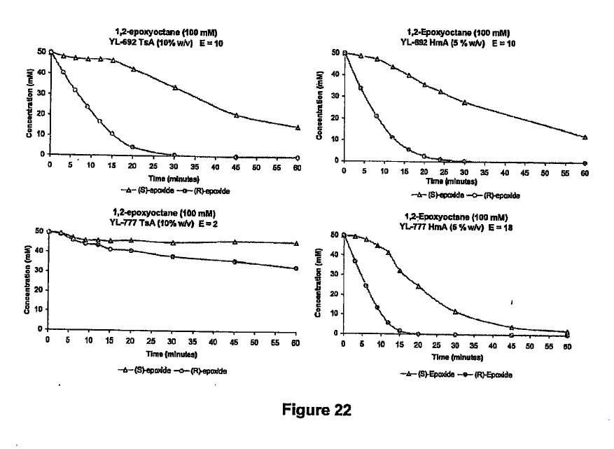

Resolution of 1,2-epoxyoctane by YL-TsA and YL-HmA transformants harboring the EH from #692 (R. paludigenum NCYC 3179) and #777 (C. neoformans CBS 132) is shown in FIG. 22. For YL-TsA transformants, 10% wet weight cells (equal to 2% dry weight) was used, while half the biomass concentration (5% wet weight=1% dry weight) was used for YL HmA transformants. For #692, the YL-HmA transformant displayed double the activity observed for the YL-TsA transformant and the selectivity remained unchanged. For # 777, an increase in both activity and selectivity of the YL-HmA transformant compared to that of the YL-TsA transformant was observed.

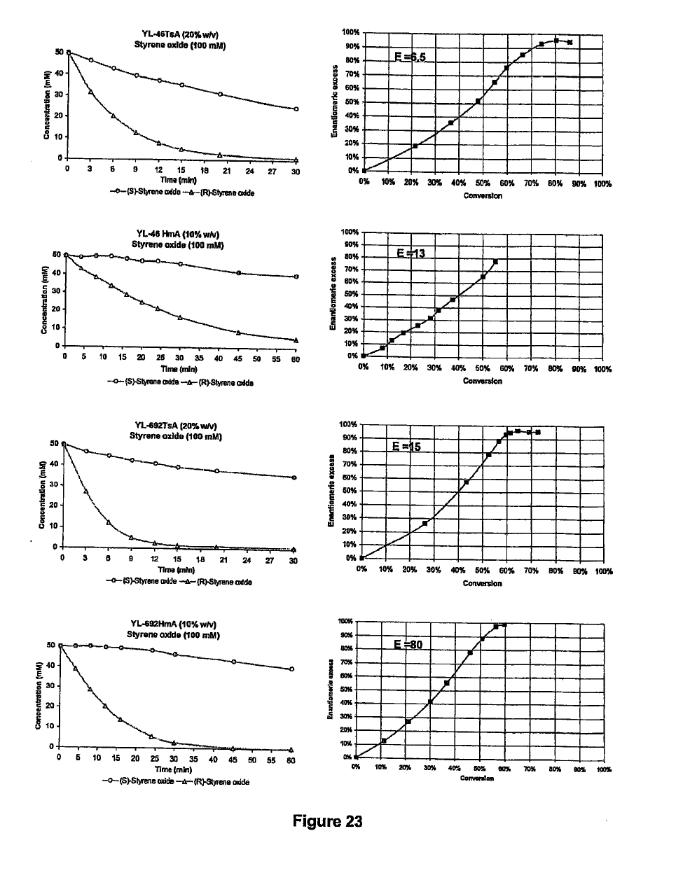

Resolution of styrene oxide by YL-TsA and YL-HmA transformants harboring the EH from #46 (R. toruloides UOFS Y-0471) and #692 (R. paludigenum NCYC 3179) is shown in FIG. 23. For YL-TsA transformants, 20% wet weight cells (equal to 4% dry weight) was used, while half the biomass concentration (10% wet weight=2% dry weight) was used for YL HmA transformants. For both #46 and #692, the activity of the YL-HmA and YL-TsA transformants remained essentially unchanged, while a significant increase in selectivity (2× for #46 and >5× for #692) was observed for both EH expressed in the YL-HmA transformants compared to the YL-TsA transformants.

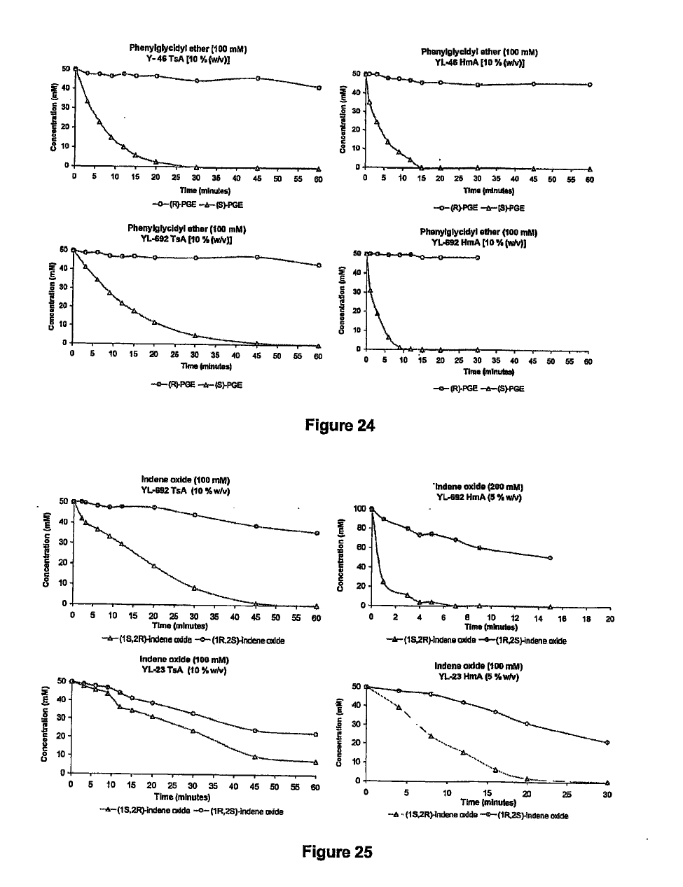

Resolution of phenyl glycidyl ether by YL-TsA and YL-HmA transformants harboring the EH from #46 (R. toruloides UOFS Y-0471) and #692 (R. paludigenum NCYC 3179) is shown in FIG. 24. For both YL-TsA and YL-HmA transformants, 10% wet weight cells (equal to 2% dry weight) was used. For both #46 and #692, the selectivity of the YL-HmA and YL-TsA transformants remained essentially unchanged, while a significant increase in activity (2× for #46 and >5× for #692) was observed for both EH expressed in the YL-HmA transformants compared to the YL-TsA transformants.

Resolution of indene oxide by YL-TsA and YL-HmA transformants harboring the EH from #692 (R. paludigenum NCYC 3179) #23 (R. mucilaginosa UOFS Y-0198) is shown in FIG. 25. For YL-TsA transformants, 10% wet weight cells (equal to 2% dry weight) was used, while half the biomass concentration (5% wet weight=1% dry weight) was used for YL HmA transformants. For #692, the YL-HmA transformant displayed 7 times the activity observed for the YL-TsA transformant and the selectivity remained essentially unchanged. For # 23, an increase in both activity and selectivity of the YL-HmA transformant compared to that of the YL-TsA transformant was observed.

In all cases, YL-HmA transformants displayed improved kinetic properties (activity and/or selectivity) compared to YL-TsA transformants.

### Example 10

### High Level Functional Expression of Cytosolic Epoxide Hydrolases from Different Sources in Yarrowia lipolytica (YL-HmA Transformants)

The epoxide hydrolase from Solanum tuberosum (potato) was selected as an example of a cytosolic EH from plant origin (Monterde et al., 2004).

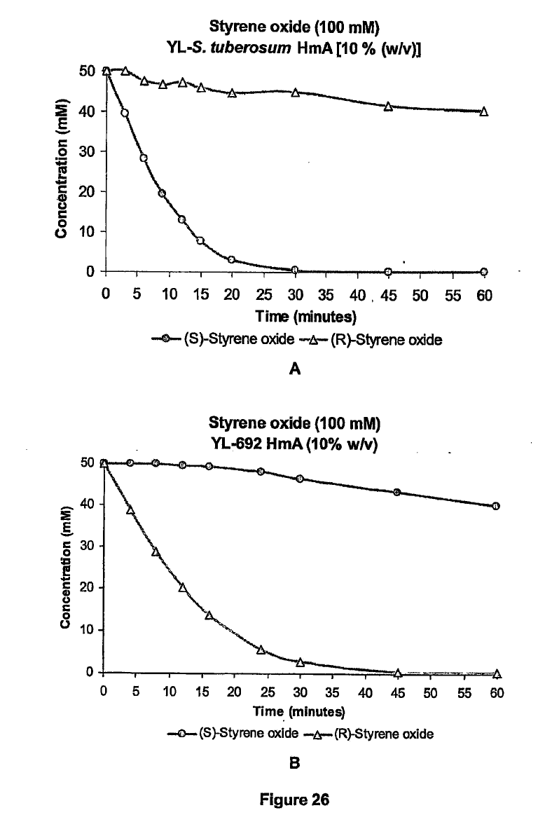

The synthesized S. tuberosum coding sequence was cloned into Y. lipolytica as described in Example 1 and the YL-St-HmA transformant was used for the hydrolysis of styrene oxide (FIG. 26A). The activity and selectivity of the recombinant potato EH enzyme was compared to that of YL-692 HmA (FIG. 26B).

Hydrolysis of styrene oxide by YL-HmA transformants harboring the coding sequences from S. tuberosum (A) and R. paludigenum (#692) (B). The S. tuberosum YL-HmA transformant displayed the same excellent enantioselectivity as reported for the native gene, which is opposite to that of yeast epoxide hydrolases. Activity was essentially identical to that obtained for YL-692HmA. Thus, it is clear that highly active and selective EH from very diverse origins can be expressed with retention of the kinetic properties in Y. lipolytica, but at much higher levels of expression.

The EH from Agrobacterium radiobacter was selected as an example of a cytosolic EH from bacterial origin (Lutje Spelberg et al., 1998). However, this enzyme reportedly became unstable if epoxide concentrations exceeded the solubility limit (i.e., formed a second phase), due to interfacial deactivation. The kinetic characteristics of this enzyme were only reported for very low concentrations (5 mM) by Spelberg et al. On the other hand, the biotransformations performed herein were at 100 mM substrate concentration.

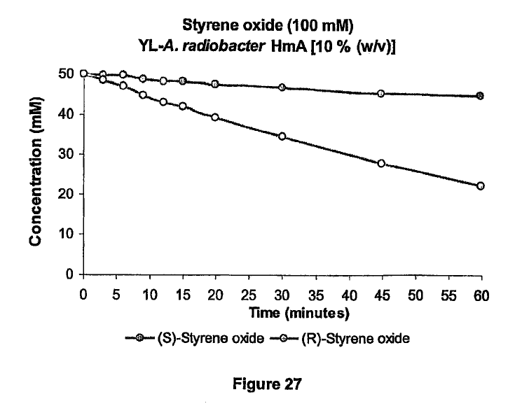

We cloned the gene from a laboratory strain of A. radiobacter and expressed the gene in Y. lipolytica as described in Example 1. The YL-Ar-HmA transformant was used for the hydrolysis of styrene oxide (FIG. 27). The selectivity compared well to published data, and no inactivation occurred when expressed intracellularly in Y. lipolytica as host.

The YL-A. radiobacter HmA transformant displayed essentially the same selectivity as reported for the native gene over-expressed in A. radiobacter.

### Example 11

### Production of Yarrowia lipolytica YL-25 HmA and Formulation as a Dry Powder Epoxide Hydrolase Biocatalyst

### Introduction

The efficient production of whole cell epoxide hydrolase biocatalyst was demonstrated using Yarrowia lipolytica recombinant strain YL-25HmA in fed-batch fermentations under a range of glucose feed rates regimes achieving a dry cell concentration of >100 g/l in less than three days fermentation duration. The strain used was constructed for intracellular production of the epoxide hydrolase under control of the quasi-constitutive hp4d promoter. The biocatalyst produced was subsequently formulated and dried using a number of different methodologies.

### Fermentative Production

### Organism Identification:

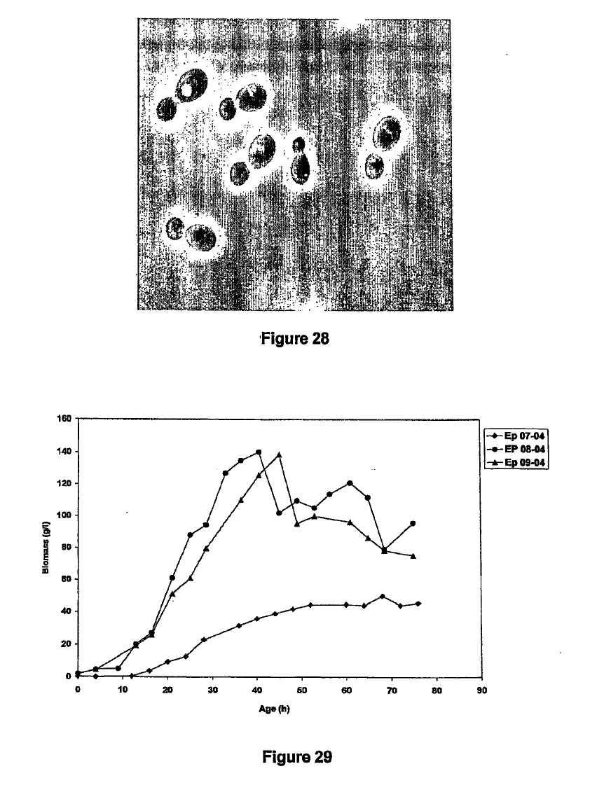

The yeast morphology is variable with normal oval shaped cells and buds to elongated pseudo-hyphal growth as shown in FIG. 28.

### Culture Maintenance:

Y. lipolytica recombinant strains were cryo-preserved in 20% glycerol and stored at −80 deg C.

### Inoculum:

The inoculum was prepared in two litre Fernbach flasks containing 10% v/v medium comprising the components listed in Table 11.

TABLE 11
Inoculum Medium

| Compound | Amount/L | Unit |
|---|---|---|
| Yeast Extract | 5 | G |
| Malt Extract | 20 | G |
| Peptone | 10 | G |
| Glucose | 15 | G |

The pH of the medium was adjusted to 5.4 with either NHOH or H₂SO₄. The flasks were inoculated with a single cryovial per flask and incubated at 28 deg C. on an orbital shaker at 150 rpm. The inoculum was transferred to the fermenters after 15-18 hours of incubation. (OD 2-8 at 660 nm).

### Production Medium:

TABLE 12
Production Medium (10 L fermenter)

| Compound | Amount/L | Unit |
|---|---|---|
| Sterilised in IC^{a} | | |
| Yeast Extract | 15 | G |
| Citric acid | 2.5 | G |
| CaCL₂•2H₂O | 0.88 | G |
| MgSO₄•7H₂O | 8.2 | G |
| NaCL | 0.1 | G |
| KH₂PO₄. | 11.3 | G |
| (NH₄)₂SO₄ | 58 | G |
| H₃PO₄ (85%) | 16.3 | Ml |
| Trace element stock solution | 1.7 | Ml |
| Antifoam | 1.00 | Ml |
| Sterilise separately | | |
| Glucose | 20 | G |
| Filter sterilised | | |
| Vitamin stocl solution | 1.7 | Ml |
| Vitamin stock solution | | |
| NaH₂PO₄•2H₂O | 0.4 | G |
| Na₂HPO₄•7H₂O | 0.2 | G |
| Meso-inositol | 100 | G |
| Nicotinic acid | 5 | G |
| Biotin | 0.2 | G |
| Thiamine HCl | 5 | G |
| Ca Panthothenate | 20 | G |
| Ascorbic | 4 | G |
| Pyridoxine HCl | 5 | G |
| Para amino beuzoic acid | 1 | G |
| Folic acid | 0.2 | G |
| Riboflavin | 0.2 | G |
| Ascorbic acid | 0.2 | G |
| Trace element stock solution | | |
| HCL | 50 | Ml |
| H₂O | 950 | Ml |
| FeSO₄•7H₂O | 35 | G |
| MnSO₄•7H₂0 | 7.5 | G |
| ZnSO₄•7H₂0 | 11 | G |
| CuSO₄•5H₂0 | 1 | G |
| CoCL₂•6H₂0 | 2 | G |
| Na₂MoO₄•2H₂0 | 1.3 | G |
| Na₂B₄O₇•10H₂0 | 1.3 | G |
| K1 | 0.35 | G |
| Al₂(SO₄)₃ | 0.5 | G |

TABLE 13
Operating parameters

| Stirrer speed (rpm) | Control stirrer to maintain 30% pO2. |
|---|---|
| Airflow (slpm) | 6 |
| Temperature (° C.) | 28 |
| pH | 5.5 (NH₄OH and H₂SO₄) |
| Pressure (mbar) | 500 |
| PO2 (%) | 30% sat |
| Inoculum volume | 3.3% |

^{a}IC is an acronym for “initial charge” and indicates the medium components that were added initially and sterilized by heat before addition of the other medium components.

### Enzyme Assay:

Enzyme assays were performed as described in Example 4 for shake flask cultures of biocatalysts on 1,2 epoxyoctane.

### Fermentation Results:

Fermentation results of three fermentations are reported in Table 14.

TABLE 14
YL-25 HmA fed-batch fermentation summary at range of glucose feed rates

| | | Glucose | Glucose | Glucose |
|---|---|---|---|---|
| | | fed at 3.8 g/ | fed at 14.5 g/ | fed at 5.0 g/ |
| | | initial | initial | initial |
| | | reactor | reactor | reactor |
| Study Description | | volume/hr | volume/hr | volume/hr |
| Age at maximum biomass | Hours | 68 | 40 | 45 |
| Maximum biomass | gram dcw/L | 44 | 140 | 138 |
| concentration | | | | |
| Max volumetric enzyme activity | mMol/min/L | 7.7 | 11-12 | 8-9 |
| (on 1,2 epoxyoctane) | | (at 68 hrs) | (>40 hrs) | (>45 hrs) |
| Max specific enzyme activity | μMol/min/g | 133.7 | 114 | 94 |
| (on 1,2 epoxyoctane) | dcw | (at 75 hrs) | (at 70 hrs) | (at 60 hrs) |

Fermentations were run to investigate the effect of different sugar feed rates on the production of the epoxide hydrolase enzyme from Yarrowia lipolytica recombinant strain YL-25 HmA. The results summarized in FIGS. 29-32.

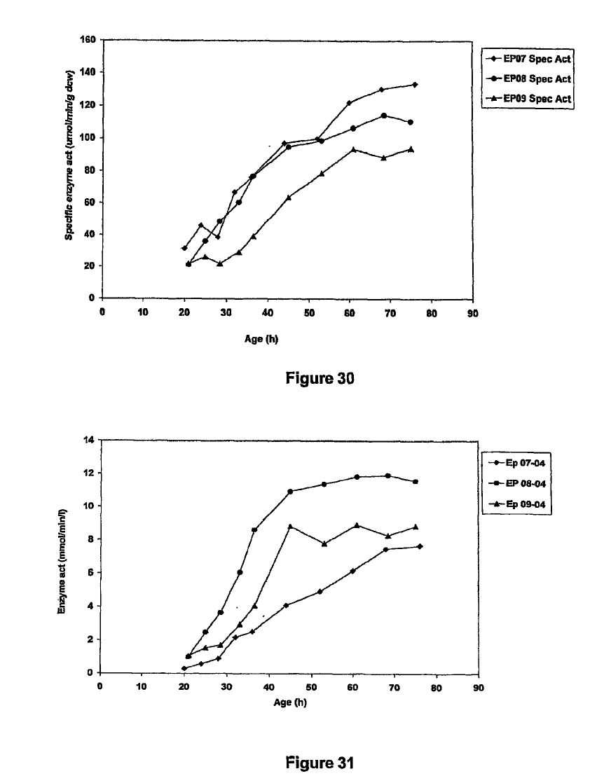

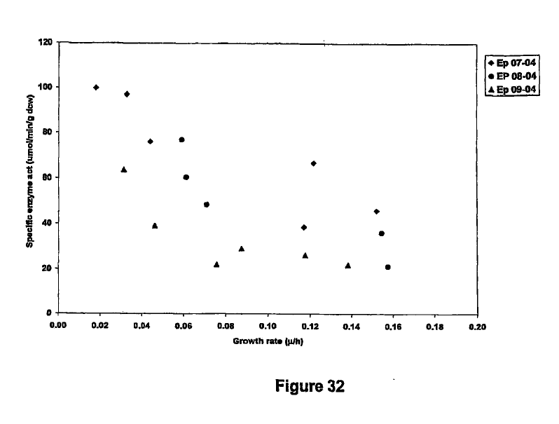

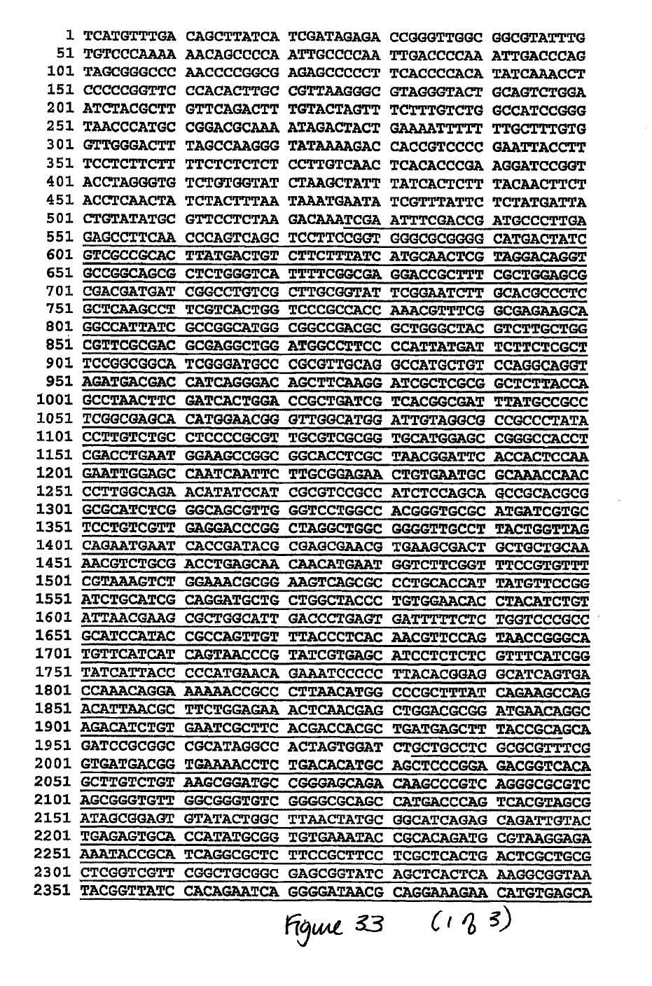

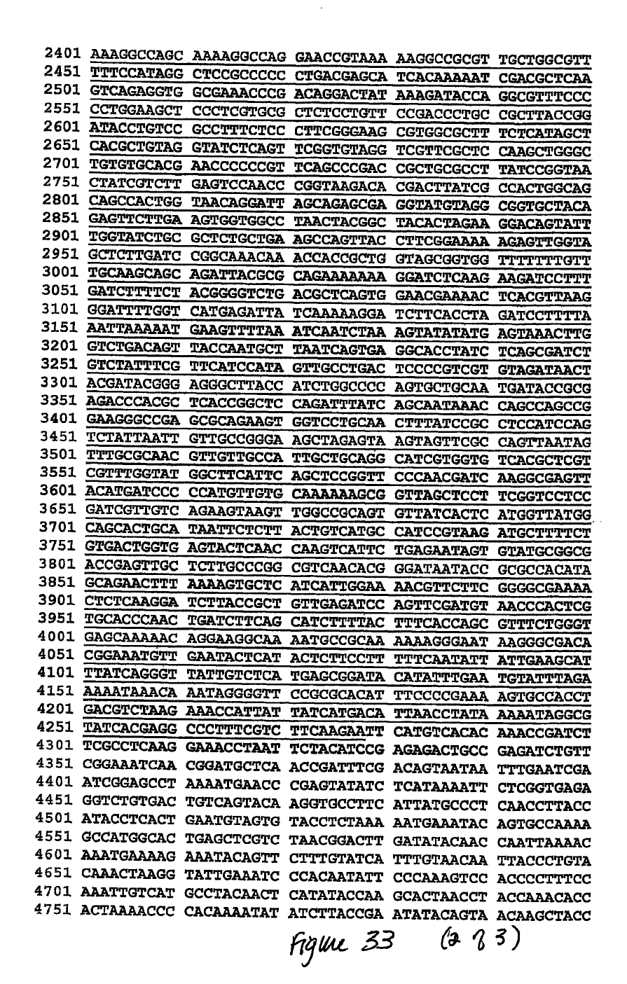

The maximum biomass specific enzyme activities obtained were 134 μMol/min/g dcw, 114 μMol/min/g dcw and of 94 μMol/min/g dcw respectively for runs for glucose feed rates of 3.8, 14.5 and 5.0 g glucose per litre initial reactor volume per hour (FIG. 30). However, due to the differences in the biomass concentrations achieved during the different fermentations, the volumetric enzyme activities were the highest at the higher glucose feed rate with decreasing volumetric activity as the feed rate decreased (FIG. 31). The main factor affecting the production of epoxide hydrolase by Y. lipolytica YL-25 HmA appeared to be the specific growth rate with the growth rate being inversely proportional to the specific enzyme activity (FIG. 32). It was evident that the specific growth rate must be maintained below 0.07 h⁻¹ for optimum biomass specific EH enzyme activity, preferably below 0.04 hr⁻¹ while still providing sufficient glucose supply for a high (>100 gram dcw per litre fermentation broth) volumetric yield of whole cell biocatalyst

### Dry Product Formulation by Fluidized Bed Drying.

Fluidised bed drying was conducted on Yarrowia lipolytica YL 25 HmA fermentation broth produced using the optimum glucose feed protocol as described above. The fermentation broth was harvested and subjected to centrifugation and washing with 50 mM phosphate buffer pH 7.5 before being centrifuged to a thick paste.

For demonstration of drying using agglomeration unit operations, the cell paste was reconstituted in 50 nM phosphate buffer pH 7.5 with and without KCl (10% m/v) to approximately 48% dry solids content. Manville Sorbocell celite (to approximately 25% of total microbial cell dry weight) was placed in the bed dryer before pumping in the slurry. The celite was used as a carrier for the yeast cells during the drying process. The slurries were pumped into the fluidised bed dryer under the following parameters:

| | | |
|---|---|---|
| Inlet temperature | 55° | C. |
| Exhaust temperature | 35-40° | C. |
| Product temperature | 40° | C. |

Each of the drying runs were conducted for approximately 1 hour. After the fluidised bed drying process, the residual water content of the 2 formulated fractions were determined by drying 1 g of each at 105° C. for 24 hours and calculating the loss in weight. The dry formulations were assayed for activity and enantioselectivity on 20 mM racemic styrene oxide using the standard biotransformation protocol and compared to the pre-dried cell broth control. The reaction was analysed by chiral gas chromatography on either an α-DEX 120 or a β-DEX 225 GC column, at 90° C. (isotherm)

For the drying protocol using the spheronisation unit operation, the cell paste was well mixed with a micro-crystalline cellulose carrier 1:1.5 (w/w) and then passed through an extruder at ambient temperature. This step yields small strips, which were then placed in a spheronizer at ambient temperature, which converts the strips into small spheres. These spheres were then placed in a fluid bed drier and dried for 1.5 hours at temperatures from 30-70° C. The final product was a powder containing viable cells with active enzyme which was assayed for water content as per the agglomeration product. The dry formulations were assayed for activity and enantioselectivity on 20 mM racemic styrene oxide using the standard biotransformation protocol and compared to the pre-dried cell broth control. The reaction was analysed by chiral gas chromatography on either an α-DEX 120 or a β-DEX 225 GC column, at 90° C. (isotherm).

TABLE 15
Effects of fluidized-bed drying on epoxide hydrolase activity and
stereoselectivity in different formulations of Yarrowia lipolytica YL-HmA whole cell
biocatalyst

| | | Retained | | |
|---|---|---|---|---|
| | Drying | activity | Retained | Water content |
| | Temperature | (% of | Enantioselectivity | after drying |
| Drying Unit operations | ° C. | Control) | (% of Control) | (% m/m) |
| Undried Control. | 4 | 100 | 100 | — |
| Fluidised bed drying after: | | | | |
| Agglomeration | | | | |
| − KCl stabiliser | 55 | 92 | 87 | 5% |
| + KCl stabiliser | 55 | 105 | 100 | 5% |
| Spheronisation | 30-60 | 70 | 100 | 3% |
| (plus MCC) | 70 | 66 | 100 | 2% |

MCC = micro-crystalline cellulose carrier

The presence of the KCl stabiliser in the agglomerated product increases both the retained activity and the retained stereoselectivity. The drying procedures demonstrated here result in a dry active powder which was found to be shelf stable for at least two weeks at ambient temperature when kept in an airtight container.

The invention includes a recombinant Yarrowia lipolytica cell able to express a polypeptide, or functional fragment thereof, having epoxide hydrolyse activity which can be used as a commercial biocatalyst having high activity and stereoselectivity while maintaining excellent stability properties both as a shelf stable biocatalyst formulation and during two phase epoxide resolution reactions. A novel highly active and stable whole cell epoxide hydrolyse biocatalyst system is provided which can be cultured to high biomass levels with an inherent high biomass-specific enzyme activity for the facile resolution of molar levels of commercially useful epoxides. An enzyme-containing biocatalyst is provided which remains active and stable for long periods and is available in a dry power catalyst form for convenient “off-the-shelf” usage for epoxide resolutions. The biocatalyst in accordance with the invention is suitable for commercial production.

Thus, the present invention includes an efficient epoxide hydrolase recombinant expression system whereby, surprisingly, the foreign coding sequence for epoxide hydrolase being derived from a yeast wild-type strain is most favourably expressed, in terms of its activity and retained high stereoselectivity, as an active intracellular polypeptide in the recombinant yeast strain Yarrowia lipolytica and in such a form the biocatalyst thereby being highly optimized for the subsequent commercial application to production of optically active epoxides (and associated vicinal diol products) in high enantiomeric purity. The invention also provides a convenient formulation of the recombinant Yarrowia lipolytica whole cell biocatalyst in a practical dry stable form while maintaining its useful kinetic characteristics.

### REFERENCES

Arand, M., Hemmer, H., Durk, H., Baratti, J., Archelas, A., Furstoss, R. and Oesch, F. (1999). Cloning and molecular characterization of a soluble epoxide hydrolase from Aspergillus niger that is related to mammalian microsomal epoxide hydrolase. Biochem. J 344, 273-280.
Arand, M., Müller, F., Mecky, A., Hinz, W., Urban, P., Pompon, D., Kellner, R. and Oesch, F. (1999b). Catalytic triad of microsomal epoxide hydrolase: replacement of Glu⁴⁰⁴ with Asp leads to a strongly increased turnover rate. Biochem. J 337, 37-43.
Barth, G. and Gaillardin, C. (1996). Non-conventional yeasts in biotechnology. A Handbook. Springer-Verlag, Berlin.
Barth, S., Fischer, M., Schmid, R. D. and Pleiss, J. (2004). Sequence and structure of epoxide hydrolases: a systematic analysis. Proteins 55, 846-855.
Bellevik, S., Zhang, J. and Meijer, J. (2002a). Brassica napus soluble epoxide hydrolase (BNSEH1). Eur. J. Biochem. 269, 5295-5302.
Bellevik, S., Summerer, S, and Meijer, J. (2002b). Overexpression of Arabidopsis thaliana soluble epoxide hydrolase 1 in Pichia pastoris and characterisation of the recombinant enzyme. Protein Expr. Purif 26, 65-70.
Buckholz, R. G. and Gleeson, M. A. G. (1991). Yeast systems for the commercial production of heterologous proteins. Bio/Technology 9, 1067-1072.
Chen, M-H., Huang, L-F., Li, H-m., Chen, Y-R. and Yu S-M. (2004) Signal Peptide-Dependent Targeting of a Rice α-Amylase and Cargo Proteins to Plastids and Extracellular Compartments of Plant Cells. Plant Physiol. 135, 1363-1377.
Enderlin, C S & Ogrydziak, D M. (1994) Cloning, nucleotide sequence and functions of XPR6, which codes for a dibasic processing endoprotease from the yeast Yarrowia lipolytica. 1994. Yeast 10:67-79.
Fabre, E., Nicaud, J-M., Lopez, M. C. and Gaillardin, C. (1991). Role of the proregion in the production and secretion of the Yarrowia lipolytica alkaline extracellular protease. J. Biol. Chem. 266, 3782-3790.
Fabre, E., Tharaud, C. and Gaillardin, C. (1992). Intracellular transit of a yeast protease is rescued by trans-complementation with its prodomain. J. Biol. Chem. 267, 15049-15055.
Gellissen, G. and Hollenberg, C. P. (1997). Application of yeasts in gene expression studies: a comparison of Saccharomyces cerevisiae, Hansenula polymorpha and Kluyveromyces lactis —a review. Gene. 190, 87-97.
Gordon, C. L., Khalaj, V., Ram, A. E. J., Archer, D. B., Brookman, J. L., Trinci, A. P. J., Jeenes, D. J., Doonan, J. H., Wells, B., Punt, P. J., Van den Hondel, C. A. M. J. J. and Robson G. D. (2000). Glucoamylase::green fluorescent protein fusions to monitor protein secretion in Aspergillus niger. Microbiology 146, 415-426.
Hill, J., Ian, K. A., Donald, D. and Griffiths, D. E. (1991) DMSO-enhanced whole cell yeast transformation. Nucl. Acids. Res. 19, 5791.
Kelly, E. J., Erickson, K. E., Sengstag, C. and Eaton, D. L. (2002). Expression of human microsomal epoxide hydrolase in Saccharomyces cerevisiae reveals a functional role in aflatoxin B1 detoxification. Toxicol. Sci. 65, 35-42.
Koschorreck, M., Fischer, M., Barth, S, and Pleiss, J. (2005). How to find soluble proteins: a comprehensive analysis of alpha/beta hydrolases for recombinant expression in E. coli. BMC Genomics 6, doi:10.1186/1471-2164-6-49.
Kronenburg, N. A. E., Mutter, M., Visser, H., De Bont, J. A. M. and Weijers, C. A. G. M. (1999). Purification of an epoxide hydrolase from Rhodotorula glutinis. Biotechnol. Lett. 21, 519-524.
Le Dall, M-T., Nicaud, J-M. and Gaillardin, C. (1994). Multi-copy integration in the yeast Yarrowia lipolytica. Curr. Genet. 26, 38-44.
Le Dall, M-T., Nicaud, J-M. and Gaillardin, C. (1994). Multi-copy integration in the yeasts Yarrowia lipolytica. Current Genetics 26, 38-44.
Lee, E. Y., Yoo, S. S., Kim, H. S., Lee, S. J., Oh, Y. K. and Park, S. (2004). Production of (S)-styrene oxide by recombinant Pichia pastoris containing epoxide hydrolase from Rhodotorula glutinis. Enzyme Microb. Technol. 35, 624-631.
Lutje Spelberg, J. H., Rink, R., Kellogg, R. M. and Janssen, D. B. (1998) Enantioselectivity of a recombinant epoxide hydrolase from Agrobacterium radiobacter. Tetrahedron: Asymmetry 9: 459-466.
Madzak, C. (2003). New tools for heterologous protein production in the yeast Yarrowia lipolytica. In: Pandalai, S. G. (Ed.), Recent Research Developments in Microbiology, vol. 7. Research Signpost, Trivandrum, pp. 453-479.
Madzak, C., Blanchin-Roland, S., Cordero Otero, R. R. and Gaillardin, C. (1999). Functional analysis of upstream regulating regions from the Yarrowia lipolytica XPR2 promoter. Microbiology 145, 75-87.
Madzak, C., Blanchin-Roland, S., Cordero-Otero, R. R. and Gaillardin, C. (1999). Functional analysis of the upstream regulating regions from the Yarrowia lipolytica XPR2 promoter. Microbiology. 145, 75-87.
Madzak, C., Tréton, B. and Blanchin-Roland, S. (2000). Strong hybrid promoters and integrative expression/secretion vectors for quasi-constitutive expression of heterologous proteins in the yeast Yarrowia lipolytica. Journal of Molecular Microbiology and Biotechnology 2, 207-216.
Matoba, S., Morano, K. A., Klionsky, D. J., Kim, K. and Ogrydziak, D. M. (1997). Dipeptidyl aminopeptidase processing and biosynthesis of alkaline extracellular protease from Yarrowia lipolytica. Microbiology. 143, 3263-3272.
Monterde, M. I., Lombard, M., Archelas, A., Cronin, A., Arand, M., Furstoss, R. (2004). Enzymatic transformations. Part 58: Enantioconvergent biohydrolysis of styrene oxide derivatives catalysed by the Solanum tuberosum epoxide hydrolase. Tetrahedron: Asymmetry 15: 2801-2805.
Morisseau, C., Archelas, A., Guitton, C., Faucher, D., Furstoss, R. and Baratti J. C. (1999). Purification and characterization of a highly enantioselective epoxide hydrolase from Aspergillus niger. Eur. J. Biochem. 263, 386-95.
Müller, S., Sandal, T., Kamp-Hansen, P., and Dalboge, H. (1998). Comparison of expression systems in the yeasts Saccharomyces cerevisiae, Hansenula polymorpha, Klyveromyces lactis, Schizosaccharomyces pombe and Yarrowia lipolytica. Cloning of two novel promoters from Yarrowia lipolytica. Yeast. 14, 1267-1283.
Murakami, Y., Philippsen, P., Tettelin, H. and Oliver, S. G. (1996). Life with 6000 genes. Science. 274, 563-567.
Nicaud J.-M., Madzak C., Van den Broek P., Gysler C., Duboc P., Niederberger P., Gaillardin C. (2002). Protein expression and secretion in the yeast Yarrowia lipolytica. FEMS Yeast Research 2, 371-279
Nicaud, J-M., Fabre, E. and Gaillardin, C. (1989b). Expression of invertase activity in Yarrowia lipolytica and its use as a selective marker. Curr. Genet. 16, 253-260.
Nicaud, J-M., Fabre, E., Beckerich, J-M., Fournier, P. and Gaillardin, C. (1989a). Cloning, sequencing and amplification of the alkaline extracellular protease XPR2 gene of the yeast Yarrowia lipolytica. J. Biotechnol. 12, 285-298.
Nicaud, J-M., Madzak, C., Van den Broek, P., Gysler, C., Duboc, P., Niederberger, P. and Gaillardin, C. (2002). Protein expression and secretion in the yeast Yarrowia lipolytica. FEMS Yeast Res. 2, 371-379.
Park, C. S., Chang, C. C. and Ryu, D. D. Y. (2000). Expression and high level secretion of Trichoderma reesei Endoglucanase I in Yarrowia lipolytica. Appl. Biochem. Biotechnol. 87, 1-15.
Park, C. S., Chang, C. C., Kim, J-Y, Ogrydziak, D. M. and Ryu, D. D. Y. (1997). Expression, secretion and processing of rice α-amylase in the yeast Yarrowia lipolytica. J. Biol. Chem. 272, 6876-6881.
Pignède, G., Wang, H-J., Fudalej, F., Seman, M., Gaillardin, C. and Nicaud, J-M. (2000). Autocloning and amplification of LIP2 in Yarrowia lipolytica. Appl. Environ. Microbiol. 66, 3283-3289.
Sambrook, J. and Russel. (2001). Molecular cloning. A laboratory manual vol. 1 (3^{rd} ed.). Cold Spring Harbor Laboratory Press. Cold Spring Harbor, N.Y.
Swennen, D., Paul, M. F., Vernis, L., Beckerich, J. M., Fournier, A. and Gaillardin, C. (2002). Secretion of active anti-Ras single-chain Fv antibody by the yeasts Yarrowia lipolytica and Kluyveromyces lactis. Microbiology. 148, 41-50.
Visser, H., de Oliveira Villela Filho, M., Liese, A., Weijers, C. A. G. M. and Verdoes, J. C. (2003). Construction and characterization of a genetically engineered E. coli strain for the epoxide hydrolase-catalyzed kinetic resolution of epoxides. Biocatal. Biotransformation 21, 33-40.
Visser, H., Weijers, C. A. G. M., Van Ooyen, A. J. J. and Verdoes, J. C. (2002). Cloning, characterization and heterologous expression of epoxide hydrolase-encoding cDNA sequences from yeast belonging to the genera Rhodotorula and Rhodosporidium. Biotechnol. Lett. 24, 1687-1694.
Watson, J. D., Hopkins, N. H., Roberts, J. W., Steitz, J. A. and Weiner, A. M. (1987). Molecular Biology of the Gene, 4^{th} ed. Benjamin Cummings, Menlo Park, Calif.
Xuan, J.-W., Fournier, P. and Gaillardin, C. (1988). Cloning of the LYS5 gene encoding saccharopine dehydrogenase from the yeast Yarrowia lipolytica by target integration. Curr. Genet. 14, 15-21.
Zhao, L., Han, B., Huang, Z., Miller, M., Huang, H., Malashock, D. S., Zhu, Z., Milan, A., Robertson, D. E., Weiner, D. P. and Burk, M. J. (2004). Epoxide hydrolase-catalyzed enantioselective synthesis of chiral 1,2-diols via desymmetrization of meso-epoxides. J. Am. Chem. Soc. 126, 11156-11157.

1. A substantially pure culture of Yarrowia lipolytica cells, a substantial number of which comprise an exogenous nucleic acid encoding an epoxide hydrolase (EH) polypeptide.

2. The substantially pure culture of cells of claim 1, wherein the exogenous nucleic acid is a vector comprising an EH polypeptide-coding sequence.

3. The substantially pure culture of cells of claim 1, wherein the EH polypeptide-coding sequence is operably linked to an expression control sequence.

4. The substantially pure culture of cells of claim 1, wherein the nucleic acid is an episome in the cells.

5. The substantially pure culture of cells of claim 1, wherein the nucleic acid is integrated into the genome of the cells.

6. The substantially pure culture of cells of claim 1, wherein the EH is a bacterial EH.

7. The substantially pure culture of cells of claim 1, wherein the EH is an insect EH.

8. The substantially pure culture of cells of claim 1, wherein the EH is a plant EH.

9. The substantially pure culture of cells of claim 1, wherein the EH is a mammalian EH.

10. The substantially pure culture of cells of claim 1, wherein the EH is a fungal EH.

11. The substantially pure culture of cells of claim 1, wherein the EH is a yeast EH.

12. The substantially pure culture of cells of claim 11, wherein the yeast is of a genus selected from the group consisting of: Arxula, Brettanomyces, Bullera, Bulleromyces, Candida, Cryptococcus, Debaryomyces, Dekkera, Exophiala, Geotrichum, Hormonema, Issatchenkia, Kluyveromyces, Lipomyces, Mastigomyces, Myxozyma, Pichia, Rhodosporidium, Rhodotorula, Sporidiobolus, Sporobolomyces, Trichosporon, Wingea, and Yarrowia.

13. The substantially pure culture of cells of claim 11, wherein the yeast is of a species selected from the group consisting of: Arxula adeninivorans, Arxula terrestris, Brettanomyces bruxellensis, Brettanomyces naardenensis, Brettanomyces anomalus, Brettanomyces species (e.g., Unidentified species NCYC 3151), Bullera dendrophila, Bulleromyces albus, Candida albicans, Candidafabianii, Candida glabrata, Candida haemulonii, Candida intermedia, Candida magnoliae, Candida parapsilosis, Candida rugosa, Candida tenuis, Candida tropicalis, Candida famata, Candida kruisei, Candida sp. (new) related to C. sorbophila, Cryptococcus albidus, Cryptococcus amylolentus, Cryptococcus bhutanensis, Cryptococcus curvatus, Cryptococcus gastricus, Cryptococcus humicola, Cryptococcus hungaricus, Cryptococcus laurentii, Cryptococcus luteolus, Cryptococcus macerans, Cryptococcus podzolicus, Cryptococcus terreus, Debaryomyces hansenii, Dekkera anomala, Exophiala dermatitidis, Geotrichum spp. (e.g., Unidentified species UOFS Y-0111), Hormonema spp. (e.g., Unidentified species NCYC 3171), Issatchenkia occidentalis, Kluyveromyces marxianus, Lipomyces spp. (e.g., Unidentified species UOFS Y-2159), Lipomyces tetrasporus, Mastigomyces philipporii, Myxozyma melibiosi, Pichia anomala, Pichia finlandica, Pichia guillermondii, Pichia haplophila, Rhodosporidium lusitaniae, Rhodosporidium paludigenum, Rhodosporidium sphaerocarpum, Rhodosporidium toruloides, Rhodosporidium paludigenum, Rhodotorula araucariae, Rhodotorula glutinis, Rhodotorula minuta, Rhodotorula minuta var. minuta, Rhodotorula mucilaginosa, Rhodotorula philyla, Rhodotorula rubra, Rhodotorula spp. (e.g., Unidentified species NCYC 3193, UOFS Y-2042, UOFS Y-0448, UOFS Y-0139, UOFS Y-0560), Rhodotorula aurantiaca, Rhodotorula spp. (e.g., Unidentified species NCYC 3224), Rhodotorula sp. “mucilaginosa”, Sporidiobolus salmonicolor, Sporobolomyces holsaticus, Sporobolomyces roseus, Sporobolomyces tsugae, Trichosporon beigelii, Trichosporon cutaneum var. cutaneum, Trichosporon delbrueckii, Trichosporon jirovecii, Trichosporon mucoides, Trichosporon ovoides, Trichosporon pullulans, Trichosporon spp. (e.g., Unidentified species NCYC 3210, NCYC 3212, NCYC 3211, UOFS Y-0861, UOFS Y-1615, UOFS Y-0451, UOFS Y-0449, UOFS Y-2113), Trichosporon moniliiforme, Trichosporon montevideense, Wingea robertsiae, and Yarrowia lipolytica.

14. The substantially pure culture of cells of claim 1, wherein the EH polypeptide is an enantioselective EH polypeptide.

15. The substantially pure culture of Yarrowia lipolytica cells of claim 1, wherein the vector comprises a constitutive promote.

16. The substantially pure culture of Yarrowia lipolytica cells of claim 15, wherein the constitutive promoter is the TEF promoter.

17. The substantially pure culture of Yarrowia lipolytica cells of claim 1, wherein the vector comprises the hp4d promoter.

18. The substantially pure culture of Yarrowia lipolytica cells of claim 5, wherein the vector integrates into the genome of the cells by a physical interaction between an integration-targeting sequence in the vector and an integration target sequence in the genomes of the cells.

19. The substantially pure culture of Yarrowia lipolytica cells of claim 18, wherein the integration-targeting sequence is an integration-targeting sequence in the pBR322 plasmid.

20. The substantially pure culture of Yarrowia lipolytica cells of claim 1, wherein the vector is the pKOV136 vector having the accession no. ______.

21. The substantially pure culture of Yarrowia lipolytica cells of claim 1, wherein the EH polypeptide is a full-length EH polypeptide.

22. The substantially pure culture of Yarrowia lipolytica cells of claim 1, wherein the EH polypeptide is a functional fragment of a full-length EH polypeptide.

23. A method of producing an EH polypeptide, the method comprising culturing the substantially pure culture of cells of claim 3 under conditions that are favorable for expression of the EH polypeptide.

24. The method of claim 23, wherein the expression results in a biomass-specific EH activity higher than the biomass-specific EH activity for cells that endogenously express the EH polypeptide.

25. The method of claim 23, wherein the EH polypeptide is substantially not secreted by the cells during the culture.

26. The method of claim 23, wherein the EH polypeptide is secreted from the cells during the culture.

27. The method of claim 23, further comprising recovering the EH polypeptide from the culture.

28. The method of claim 27, wherein the EH polypeptide is recovered from the cultured cells.

29. The method of claim 27, wherein the EH polypeptide is recovered from the medium in which the cells are cultured.

30. A substantially pure composition of dry Yarrowia lipolytica cells, a substantial number of which comprise an exogenous nucleic acid encoding an EH polypeptide.

31. The composition of claim 30, wherein the composition is made dry using a method selected from the group consisting of freeze-drying, spray drying, fluidized bed drying, and agglomeration.

32. The composition of claim 30, wherein the composition is a shelf-stable, dry biocatalyst composition suitable for biocatalytic resolution of racemic epoxides.

33. The composition of claim 30, wherein the cells were co-formulated with one or more stabilizing agents prior to drying.

34. The composition of claim 33, wherein the one or more of the stabilizing agents is a salt.

35. The composition of claim 33, wherein the one or more of the stabilizing agents is a sugar.

36. The composition of claim 33, wherein the one or more of the stabilizing agents is a protein.

37. The composition of claim 33, wherein the one or more of the stabilizing agents is an inert carrier.

38. The composition of claim 33, wherein one of the stabilizing agents is KCl.

39. A method of hydrolysing an epoxide, the method comprising:
providing an epoxide sample;
creating a reaction mixture by mixing a Y. lipolytica cellular EH biocatalytic agent with the epoxide sample; and
incubating the reaction mixture.

40. The method of claim 39, wherein the epoxide sample is a enantiomeric mixture of an optically active expoxide and the Y. lipolytica cellular EH biocatalytic agent is enantioselective.

41. The method of claim 40, further comprising recovering from the reaction mixture: (a) an enantiopure, or a substantially enantiopure, vicinal diol; (b) an enantiopure, or a substantially enantiopure, epoxide; or (c) an enantiopure, or a substantially enantiopure, vicinal diol and an enantiopure, or a substantially enantiopure, epoxide.

42. The method of claim 40, wherein the optically active epoxide is an epoxide selected from the group consisting of monosubstituted epoxides, styrene oxides, 2,2-disbubstituted epoxides, 2,3-disbubstituted epoxides, trisubstituted epoxides, tetra-substituted epoxides, meso-epoxides, and glycidyl ethers.

43. The method of claim 39, wherein the Y. lipolytica cellular EH biocatalytic agent is a substantially pure population of Yarrowia lipolytica cells, a substantial number of which comprise an exogenous nucleic acid encoding an EH polypeptide.

44. The method of claim 40, wherein the Y. lipolytica cellular EH biocatalytic agent is a lysate or extract of a substantially pure population of Yarrowia lipolytica cells, a substantial number of which comprise an exogenous nucleic acid encoding an EH polypeptide.

45. A vector comprising:
an expression control sequence;
a constitutive promoter; and
an integration-targeting sequence.

46. The vector of claim 45, wherein the constitutive promoter is the TEF promoter.

47. The vector of claim 45, wherein the integration-targeting sequence comprises a nucleotide sequence from the pBR322 plasmid.

48. The vector of claim 47, wherein the nucleotide sequence is the entire or partial nucleotide sequence of the pBR322 plasmid.

49. The vector of claim 45, wherein the vector is the PKOV136 vector having accession number ______.

50. An isolated Yarrowia lipolytica cell comprising an exogenous nucleic acid encoding an epoxide hydrolase (EH) polypeptide.
